(19) United States
(12) Patent Application Publication
(10) Pub. No.: US 2025/0249197 A1
(43) Pub. Date: Aug. 7, 2025

(54) MEDICAL GASES CONDUIT

(71) Applicant: Fisher & Paykel Healthcare Limited, Auckland (NZ)

(72) Inventors: Kieran James HATCHER, Auckland (NZ); Anthony James NEWLAND, Auckland (NZ); Bhuvan GARG, Auckland (NZ); Leo Mckenzie RODGER, Auckland (NZ); Thomas James EDWARDS, Auckland (NZ)

(21) Appl. No.: 18/854,917

(22) PCT Filed: Apr. 10, 2023
(86) PCT No.: PCT/NZ2023/050040

§ 371 Date: Oct. 7, 2024

Related U.S. Application Data
(60) Provisional application No. 63/329,205, filed on Apr. 8, 2022.

(51) Int. Cl. A61M 16/08; A61M 13/00; A61M 16/10; A61M 16/16

(52) U.S. Cl. CPC A61M 16/0808; A61M 13/003; A61M 16/0833; A61M 16/0883; A61M 16/1095; A61M 16/16; A61M 2205/7536; A61M 2207/00

(57) ABSTRACT

A conduit for conveying medical gases includes an elongate tube, a sheath provided about the elongate tube, and a pair of connectors provided at opposing ends of the elongate tube. The elongate tube is formed from a breathable material to mitigate condensate within the lumen. In use, water molecules within the lumen of the elongate tube are dissipated by absorption into the breathable material, diffusion through the breathable material, and desorption to ambient air. The breathable material may tend to expand and soften upon absorbing water molecules. Expansion of the elongate tube may be selectively constrained by the sheath. Constraint by the sheath may mitigate the softening of the breathable material.

This application claims the benefit of priority from U.S. Provisional Patent Application No. 63/329,205, entitled “RESPIRATORY CONDUIT,” which is incorporated herein by reference in its entirety.

### BACKGROUND

### Field

The present disclosure relates to conduits for use in conveying medical gases. More particularly, though not exclusively, the disclosure relates to a conduit including a breathable material which is permeable to water molecules, for use in a respiratory breathing circuit of a respiratory assistance system or an insufflation circuit of a surgical insufflation system.

### Description of Related Art

It has been shown that, for patients receiving respiratory assistance, there are clinical benefits when the temperature and humidity of respiratory gases supplied to the patient by a respiratory assistance system emulate the levels that occur naturally in healthy lungs (generally about 37° Celsius and 100% relative humidity). The respiratory gases may therefore be heated and humidified for improved patient comfort and recovery.

Similarly, there are clinical benefits to heating and humidifying an insufflation gas supplied to a patient's abdominal or peritoneal cavity by a surgical insufflation system, e.g., during a laparoscopic procedure. The insufflation gas may be supplied from a cylinder stored at room temperature (e.g., between about 19 and 21 degrees Celsius), with a relative humidity approaching 0%. Heating and humidifying the insufflation gas can decrease cellular damage or desiccation, limit adhesion formation, or reduce other deleterious effects.

In medical gases systems (e.g., respiratory assistance systems or surgical insufflation systems), medical gases (respiratory gases or insufflation gas, respectively) may be conveyed to and from the patient by conduits. A conduit has a tube wall defining a lumen for passage of the medical gases. The exterior surface of the tube wall is generally exposed to ambient air surrounding the exterior surface of the conduit. The ambient air is generally at a relatively lower temperature and humidity than the heated and humidified flow of medical gases within the lumen. In a hospital environment, for example, one or more of the temperature and humidity of ambient air is commonly regulated by hospital heating, ventilation and air conditioning (HVAC) systems for comfort and air quality. In other environments, such as the patient's home, one or more of the temperature and humidity of ambient air may be either regulated or unregulated. The temperature and humidity of the ambient air may fluctuate widely throughout the day and seasons, particularly if they are not regulated.

The temperature difference between the medical gases and ambient air causes the heated and humidified medical gases to cool as they pass along the length of the conduit. If the temperature of the medical gases falls below the dew point, water vapor in the flow of medical gases will condense into liquid water droplets.

Condensate may also form upstream and downstream of the conduit. For example, in a respiratory assistance system, the high humidity of respiratory gases conveyed to a ventilator or anesthesia machine by an expiratory conduit may cause condensate to form within the ventilator or anesthesia machine. Condensate may accumulate in a filter or on a flow sensor within the ventilator or anesthesia machine.

Condensate and other liquids may drain into one or more of the conduits from other sources, e.g., one or more of a nebulizer, Y-piece, catheter mount, patient interface, patient, and the like.

Accumulated condensate or other liquids in the conduits or other components of the medical gases system may cause a variety of problems, such as one or more of:
false sensor readings;
saturation of filters;
alarms (e.g., audible and visual);
damage to the flow source or components thereof (e.g., flow sensors);
the need for periodic draining which may interrupt the surgery or therapy; and
occlusion of the flow path.

### BRIEF SUMMARY

In a first aspect, a medical gases conduit for use in a medical gases system comprises: an elongate tube defining a lumen for passage of a flow of medical gases, at least a portion of the elongate tube comprising a breathable material which, in use, is configured to expand due to absorption of water molecules; and a sheath provided about at least a portion of the elongate tube, wherein the sheath is configured to constrain, in use, the expansion of at least a portion of the elongate tube in at least one of a radial direction and a longitudinal direction due to the absorption of the water molecules by the breathable material.

In a second aspect, a medical gases conduit for use in a medical gases system comprises: an elongate tube defining a lumen for passage of a flow of medical gases, at least a portion of the elongate tube being corrugated and comprising: a breathable material which, in use, is configured to expand due to absorption of water molecules, and a plurality of corrugations; and a sheath provided about at least a portion of the elongate tube and configured to, in use, constrain expansion of the elongate tube in at least a longitudinal direction of the elongate tube; wherein the elongate tube is configured so that a pitch of the plurality of corrugations varies in use, in dependence upon the absorption of water molecules by the breathable material and constraint of the elongate tube by the sheath.

In a third aspect, a medical gases conduit for use in a medical gases system comprises: an elongate tube defining a lumen for passage of a flow of medical gases, at least a portion of the elongate tube comprising a breathable material which, in use, is configured to expand due to absorption of water molecules; and a sheath provided about at least a portion of the elongate tube, each end of the sheath secured to a corresponding end of the elongate tube, wherein the sheath is configured so that a length and a diameter of the sheath vary in inverse relation to each other as the breathable material expands due to the absorption of the water molecules, in use.

In a fourth aspect, a medical gases conduit for use in a medical gases system comprises: an elongate tube defining a lumen for passage of a flow of medical gases; a sheath provided about at least a portion of the elongate tube; and a pair of connectors for pneumatically coupling the elongate tube to other components of the respiratory system, each of the pair of connectors securing a respective end of the sheath relative to a corresponding end of the elongate tube, and at least a first connector of the pair of connectors comprising a plurality of apertures through which one or more of the elongate tube and the sheath are exposed.

In a fifth aspect, a medical gases conduit for use in a medical gases system comprises: an elongate tube defining a lumen for passage of a flow of medical gases, and a sheath provided about at least a portion of the elongate tube, wherein the sheath is configured to be at least partially spaced from a maximum outer diameter of the elongate tube when the medical gases conduit is in an equilibrated state.

In a sixth aspect, a medical gases conduit for use in a medical gases system comprises: an elongate tube defining a lumen for passage of a flow of medical gases, at least a portion of the elongate tube comprising a breathable material which, in use, is configured to expand upon absorption of water molecules; and a sheath provided about at least a portion of the elongate tube and configured to selectively constrain expansion of the elongate tube due to the absorption of the water molecules, in use; wherein the elongate tube and the sheath are configured so that, in at least some conditions, a compliance of the medical gases conduit is dependent on a physical interaction between the elongate tube and the sheath.

In a seventh aspect, a medical gases conduit for use in a medical gases system comprises: an elongate tube defining a lumen for passage of a flow of medical gases, at least a portion of the elongate tube comprising a breathable material which, in use, is configured to expand upon absorption of water molecules; and a sheath provided loosely about at least a portion of the elongate tube; wherein the elongate tube is configured to exert a hoop stress upon the sheath in at least some conditions, in use.

In an eighth aspect, a medical gases conduit for use in a medical gases system comprises: an elongate tube defining a lumen for passage of a flow of medical gases; and a sheath provided about at least a portion of the elongate tube, the sheath comprising a braided tubular mesh, the braided tubular mesh comprising a biaxial braid of a plurality of braiding elements, the plurality of braiding elements being spaced about a circumference of the sheath and extending helically about the elongate tube, and the plurality of braiding elements each comprising a pair of adjacent filaments.

In a ninth aspect, a medical gases conduit for use in a medical gases system comprises an elongate tube defining a lumen for passage of a flow of medical gases, at least a portion of the elongate tube comprising a breathable material, at least a portion of the elongate tube is configured to absorb between about 100% and 250%, preferably between about 100% and 180%, more preferably between about 110% and 150%, yet more preferably between about 120% and 140% or between about 130% and 145%, and yet more preferably between about 135% and 145%, e.g., about 138% or 139%, of its own dry mass in water molecules in immersion testing.

In a tenth aspect, a medical gases conduit for use in a medical gases system comprises an elongate tube defining a lumen for passage of a flow of medical gases, at least a portion of the elongate tube comprising a breathable material, at least a portion of the elongate tube is configured to absorb between about 40% and 80%, preferably between about 40% and 60%, more preferably between about 45% and 55%, and yet more preferably between about 48% and 51%, e.g., about 49%, of its own dry mass in water molecules in immersion testing, and the medical gases conduit does not comprise a heater wire.

In an eleventh aspect, a medical gases conduit for use in a medical gases system comprises: an extruded elongate tube defining a lumen for passage of a flow of medical gases, at least a portion of the elongate tube comprising a breathable material; and a pair of connectors at respective ends of the elongate tube for pneumatically coupling the medical gases conduit with other components of the medical gases system, at least one of the pair of connectors comprising: a first connector component inserted at least partially within the lumen of the elongate tube, and a second connector component overmolded to the first connector component and the elongate tube.

In a twelfth aspect, a medical gases conduit for use in a medical gases system comprises an elongate tube defining a lumen for passage of a flow of medical gases, at least a portion of the elongate tube comprising a breathable material, the breathable material in differential scanning calorimetry (DSC) testing comprising two melting points each at temperatures above about 197° C., preferably above about 202° C., more preferably above about 204° C., yet more preferably above about 205° C., and most preferably above about 206° C., e.g., at or above a temperature of about 207° C., wherein the medical gases conduit does not comprise a heater wire.

In a thirteenth aspect, a medical gases conduit for use in a medical gases system comprises an elongate tube defining a lumen for passage of a flow of medical gases, at least a portion of the elongate tube comprising a breathable material, the breathable material in differential scanning calorimetry (DSC) testing comprising three melting points, wherein the medical gases conduit does not comprise a heater wire.

In a fourteenth aspect, a medical gases conduit for use in a medical gases system comprises an elongate tube defining a lumen for passage of a flow of medical gases, at least a portion of the elongate tube comprising a breathable material, the breathable material in differential scanning calorimetry (DSC) testing comprising a lowest melting point at a temperature greater than about 37° C., preferably greater than about 42° C., more preferably greater than about 44° C., more preferably greater than about 45° C., and yet more preferably greater than about 46° C., e.g., at a temperature of about 47° C.

In a fifteenth aspect, a medical gases conduit for use in a medical gases system comprises an elongate tube defining a lumen for passage of a flow of medical gases, at least a portion of the elongate tube comprising a breathable material which, in use, is configured to expand due to absorption of water molecules, wherein the medical gases conduit is configured so that the elongate tube, in at least one conditioned state in use, has localized expansion in one or more of: an inlet region, the inlet region comprising up to about 50%, preferably up to about 33%, more preferably up to about 25%, yet more preferably up to about 20%, and most preferably up to about 10% of a length of the elongate tube nearest a patient interface of the medical gases system; an outlet region, the outlet region comprising up to about 50%, preferably up to about 33%, more preferably up to about 25%, yet more preferably up to about 20%, and most preferably up to about 10% of the length of the elongate tube nearest a gases return inlet of a gases source of the medical gases system; and an intermediate region, the intermediate region comprising up to about 50%, preferably up to about 33%, more preferably up to about 25%, yet more preferably up to about 20%, and most preferably up to about 10% of the length of the elongate tube intermediate the inlet region and the outlet region.

The medical gases conduit of any one of the ninth to fifteenth aspects may comprise a sheath provided about at least a portion of the elongate tube.

The medical gases conduit of any one of the fourth, fifth or eighth aspects may comprise an elongate tube comprising a breathable material.

The following technical features may apply to the medical gases conduit of any one of the first to fifteenth aspects.

The medical gases conduit may be an expiratory conduit, the medical gases system may be a respiratory assistance system, and the flow of medical gases may be a flow of respiratory gases. Or the medical gases conduit may be an inspiratory conduit, the medical gases system may be a respiratory assistance system, and the flow of medical gases may be a flow of respiratory gases. Or the medical gases conduit may be a discharge conduit, the medical gases system may be a surgical insufflation system, and the flow of medical gases may be a flow of insufflation gas.

A diameter and a length of at least one, and preferably both, of the elongate tube and the sheath may vary temporally, in use.

A length and a diameter of the sheath may be configured to vary in inverse relation as the elongate tube expands due to absorption of water molecules, in use.

A length vs diameter gradient of the sheath may be between about −100 and −20, preferably between about −50 and −20, more preferably between about −40 and −35, and is most preferably about −37. Or a length vs diameter gradient of the sheath may be between about −75 and −25, preferably between about −60 and −40, more preferably between about −55 and −45, and is most preferably about −52.

A length vs diameter gradient of the sheath may be substantially linear between sheath diameters of about 24 mm and 32 mm.

The sheath, in isolation, may be configured to conform to rods with outside diameters ranging between about 23 mm and 43 mm.

The sheath may comprise a plurality of openings, preferably quadrilateral openings, which are configured to undergo an affine transformation as the elongate tube expands due to the absorption of the water molecules by the breathable material, in use.

The sheath may be configured to be at least partially spaced from the elongate tube when the medical gases conduit is in at least one, and preferably both, of a dry state and an equilibrated state.

The elongate tube and the sheath may be configured so that, in at least one cross-section through the medical gases conduit, the sheath is spaced outwardly from at least part of the circumference of the elongate tube in any one or more of a dry state, an equilibrated state, and at least one conditioned state, in use.

The elongate tube and the sheath may be configured so that the elongate tube exerts a hoop stress upon the sheath in at least one conditioned state, in use.

The elongate tube and the sheath may be configured so that, in at least one cross-section through the medical gases conduit, the sheath contacts an entire circumference of the elongate tube in at least one conditioned state, in use.

The breathable material may be unfoamed. Or the breathable material may be foamed.

The breathable material may comprise a block copolymer, preferably a block copolymer comprising one or more of: hard segments of polybutylene terephthalate; and soft segments of an ether type macro glycol.

The block copolymer may comprise at least about 90%, preferably at least about 92%, more preferably at least about 95%, yet more preferably at least about 97%, e.g., about 97% or about 98.5% of the breathable material by one or more of mass, weight and volume.

The breathable material may comprise one or more additives, e.g., one or more of: a foaming agent, a colorant, an ultraviolet (UV) stabilizer, a UV absorber, and a processing aid. The one or more additives may comprise up to about 10%, preferably up to about 8%, more preferably up to about 5%, and most preferably up to about 3%, e.g., about 3% or about 2% of the breathable material, by one or more of mass, weight and volume.

At least a portion of the elongate tube may be configured to absorb more than about 33%, more preferably between about 33% and 200%, yet more preferably between about 100% and 160%, yet more preferably between about 120% and 140%, and most preferably between about 130% and 135%, e.g., about 133%, of its own dry mass in water molecules, in immersion testing.

At least a portion of the elongate tube may be configured to absorb between about 45% and 250%, preferably between about 65% and 200%, more preferably between about 75% and 175%, yet more preferably between about 100% and 160%, yet more preferably between about 110% and 150%, yet more preferably between about 120% and 140%, yet more preferably between about 130% and 140%, and most preferably between about 133% and 139%, e.g., about 139%, of its own dry mass in water molecules, in immersion testing.

In immersion testing in isolation from the sheath, at least a portion of the elongate tube may be configured to expand by between about 20% and 70%, preferably between about 25% and 50%, and more preferably between about 30% and 50%, e.g., by about 32%, in one or more, and preferably each, of the radial direction, the longitudinal direction, and a wall thickness in immersion testing.

In immersion testing in isolation from the sheath, at least a portion of the elongate tube may be configured to expand by one or more, and preferably all, of: between about 37% and 47%, preferably about 42%, in the radial direction; between about 32% and 42%, preferably about 37%, in the longitudinal direction; and/or between about 29% and 39%, preferably about 34%, in the wall thickness.

The medical gases conduit may comprise a pair of connectors at opposing ends of the medical gases conduit, the pair of connectors configured for pneumatically coupling the elongate tube with other components of the medical gases system, at least one of the pair of connectors comprising a first connector component and a second connector component, wherein corresponding ends of the elongate tube and the sheath are secured between the first connector component and the second connector component.

The first connector component may extend into the lumen of the elongate tube.

The second connector component may be overmolded to one or more, and preferably all, of the first connector component, the elongate tube and the sheath.

The second connector component may comprise a plurality of apertures, preferably between two and six apertures, e.g., four apertures, through which the elongate tube and the sheath are exposed.

An end of the elongate tube may be disposed substantially concentrically about a portion of the first connector component; an end of the sheath may be disposed substantially concentrically about the end of the elongate tube; and the second connector component may be disposed substantially concentrically about the end of the sheath.

At least one of the first connector component and the second connector component may be formed at least in part from one or more of polycarbonate, polypropylene and polyethylene.

At least one of the pair of connectors may be configured to prevent or inhibit expansion of the end of the elongate tube secured between the first connector component and the second connector component.

The elongate tube and the sheath may be not secured to each other intermediate the pair of connectors.

The conduit may be configured so that the elongate tube tapers towards at least one, and preferably both, of the pair of connectors in at least one conditioned state, in use.

The conduit may be configured so that the sheath tapers towards at least one, and preferably both, of the pair of connectors in at least one of, and preferably both, an equilibrated state and a conditioned state.

The medical gases conduit may be configured so that, in use: upon initially absorbing water molecules, the elongate tube is configured to expand relatively unconstrained by the sheath, and upon further absorption of water molecules, the elongate tube is configured to expand relatively constrained by the sheath.

The elongate tube may be configured to expand relatively constrained by the sheath after prolonged use.

The sheath may be configured to: not constrain expansion of the elongate tube when the elongate tube is in an equilibrated state, in use; and constrain expansion of the elongate tube when the elongate tube is in at least one conditioned state, in use.

The medical gases conduit may be configured so that, in use: upon initially absorbing water molecules, the elongate tube is configured to expand in the radial direction relatively unconstrained by the sheath, and upon further absorption of water molecules, the elongate tube is configured to further expand in the radial direction relatively constrained by the sheath.

The sheath may be configured to: not constrain expansion of the elongate tube in the radial direction when the elongate tube is in an equilibrated state; and constrain expansion of the elongate tube in the radial direction when the elongate tube is in at least one conditioned state, in use.

The medical gases conduit may be configured so that, in use: upon initially absorbing water molecules, the elongate tube is configured to expand in the longitudinal direction relatively unconstrained by the sheath; and upon further absorption of water molecules, the elongate tube is configured to expand in the longitudinal direction relatively constrained by the sheath.

The medical gases conduit may be configured so that, upon yet further absorption of water molecules in use, the elongate tube is configured to expand in the radial direction and the sheath is configured to cause the medical gases conduit to at least partially contract in the longitudinal direction.

The sheath may be configured to not constrain expansion of the elongate tube in the longitudinal direction in an equilibrated state, and to constrain expansion of the elongate tube in the longitudinal direction in at least one conditioned state.

The medical gases conduit may be configured so that, in use: upon initially absorbing water molecules, the elongate tube is configured to freely expand in the radial direction and the longitudinal direction, and upon further absorption of water molecules, the elongate tube is configured to engage the sheath and further expansion is constrained in at least one of the radial direction and the longitudinal direction.

The sheath may be configured to allow the elongate tube to expand freely in the radial direction and the longitudinal direction in an equilibrated state, and configured to constrain expansion of the elongate tube in at least one of the radial direction and the longitudinal direction in at least one conditioned state.

The medical gases conduit may be configured so that, in use: upon initially absorbing water molecules, the elongate tube is configured to freely expand in a radial direction to engage an inner surface of the sheath, and upon further absorption of water molecules, the elongate tube is configured to further expand in the radial direction, causing the sheath to expand in the radial direction and contract in the longitudinal direction.

The elongate tube and the sheath may be configured so that expansion of the elongate tube in the radial direction causes the elongate tube to engage the sheath, and the sheath to expand in the radial direction and contract in the longitudinal direction.

The elongate tube may be configured to have a first length prior to use, a second length upon initially absorbing water molecules, and a third length upon further absorption of water molecules, wherein the second length is longer than the first length and the third length.

The elongate tube may be configured to have a first length in an equilibrated state, a second length in at least one conditioned state in which radial expansion of the elongate tube is not constrained by the sheath, and a third length in at least one other conditioned state in which radial expansion of the elongate tube is constrained by the sheath, wherein the second length is longer than the first length and the third length.

The third length may be within about 90% to 110% of the first length, and preferably about equal to the first length.

The elongate tube may comprise a plurality of corrugations.

The medical gases conduit may be configured so that a profile of each of the plurality of corrugations changes as the elongate tube expands due to absorption of water molecules, in use.

The plurality of corrugations may be each defined in part by a pair of side walls, and the elongate tube and the sheath may be configured so that the side walls reorient, preferably towards the radial direction, as the elongate tube absorbs water molecules, in use.

The side walls may be configured to reorient beyond a radial direction as the elongate tube continues to absorb water molecules.

The medical gases conduit may be configured so that a pitch of the plurality of corrugations is substantially uniform along a length of the elongate tube when the elongate tube is in at least one of a dry state and an equilibrated state, and the pitch of the plurality of corrugations varies along the length of the elongate tube when the elongate tube is in at least one conditioned state.

The elongate tube may comprise a cuff portion at an end of the elongate tube, wherein the cuff portion is uncorrugated.

The sheath may comprise a tubular mesh, preferably a braided tubular mesh.

The sheath may comprise a biaxial braid of a plurality of braiding elements each extending helically about the elongate tube.

The plurality of braiding elements may each comprise two or three filaments.

The sheath may comprise at least one of: between about 75 and 125 braiding elements, preferably between about 90 and 100 braiding elements, e.g., about 96 braiding elements; and between about 150 and 250 filaments, and preferably between about 180 and 200 filaments, e.g., about 192 filaments.

The elongate tube may be configured to have: a substantially uniform diameter in at least one of a dry state and an equilibrated state; and a substantially non-uniform diameter in at least one conditioned state, in use.

The medical gases conduit may be configured so that the elongate tube, in at least one conditioned state in use, has localized expansion in one or more regions of elevated relative humidity or volume of condensate within the lumen.

The medical gases conduit may be an expiratory conduit configured so that the elongate tube, in a conditioned state in use, has localized expansion in one or more of: an inlet region, the inlet region comprising up to about 50%, preferably up to about 33%, more preferably up to about 25%, yet more preferably up to about 20%, and most preferably up to about 10% of a length of the elongate tube nearest a patient interface of the medical gases system; an outlet region, the outlet region comprising up to about 50%, preferably up to about 33%, more preferably up to about 25%, yet more preferably up to about 20%, and most preferably up to about 10% of the length of the elongate tube nearest a gases return inlet of a gases source of the medical gases system; and an intermediate region, the intermediate region comprising up to about 50%, preferably up to about 33%, more preferably up to about 25%, yet more preferably up to about 20%, and most preferably up to about 10% of the length of the elongate tube intermediate the inlet region and the outlet region.

The breathable material in differential scanning calorimetry (DSC) testing may comprise a lowest melting point at a temperature greater than about 37° C., preferably greater than about 42° C., more preferably greater than about 44° C., more preferably greater than about 45° C., and most preferably greater than about 46° C., e.g., at a temperature of about 47° C.

The breathable material in DSC testing may comprise a lowest melting point at a temperature between about 37° C. and 100° C., preferably between about 40° C. and 60° C., and more preferably between about 40° C. and 50° C., e.g., at a temperature of about 47° C.

The breathable material in differential scanning calorimetry (DSC) testing may comprise a melting point at a temperature between about 42° C. and 52° C., preferably between about 44° C. and 50° C., more preferably between about 45° C. and 49° C., and yet more preferably between about 46° C. and 47° C., e.g., at a temperature of about 47° C.

The breathable material in differential scanning calorimetry (DSC) testing may comprise two melting points at temperatures above about 197° C., preferably above about 202° C., more preferably above about 204° C., yet more preferably above about 205° C., and most preferably above about 206° C., e.g., at or above a temperature of about 207° C.

The breathable material in differential scanning calorimetry (DSC) testing may comprise two melting points at temperatures between about 202° C. and 225° C., preferably between about 204° C. and 223° C., more preferably between about 205° C. and 222° C., and yet more preferably between about 206° C. and 221° C., e.g., at temperatures of about 207° C. and 220° C.

The breathable material in differential scanning calorimetry (DSC) testing may comprise two melting points with a temperature difference of less than about 23° C., preferably less than about 18° C., more preferably less than about 16° C., yet more preferably less than about 15° C., and most preferably less than about 14° C., e.g., with a temperature difference of about 13° C.

The breathable material in differential scanning calorimetry (DSC) testing may comprise two melting points with a temperature difference of between about 150° C. and 170° C., preferably between about 155° C. and 165° C., more preferably between about 158° C. and 162° C., and yet more preferably between about 159° C. and 161° C., e.g., a temperature difference of about 160° C.

The breathable material in differential scanning calorimetry (DSC) testing may comprise three melting points at temperatures between about 42° C. and 225° C., more preferably between about 44° C. and 223° C., yet more preferably between about 45° C. and 222° C., and most preferably between about 46° C. and 221° C.

The breathable material in differential scanning calorimetry (DSC) testing may comprise melting point(s) at temperature(s) of at least one, and preferably all three, of: between about 42° C. and 52° C., preferably between about 44° C. and 50° C., more preferably between about 45° C. and 49° C., and yet more preferably between about 46° C. and 47° C., e.g., at a temperature of about 47° C.; between about 202° C. and 212° C., preferably between about 204° C. and 210° C., more preferably between about 205° C. and 209° C., and yet more preferably between about 206° C. and 208° C., e.g., at a temperature of about 207° C.; and between about 215° C. and 225° C., preferably between about 217° C. and 223° C., more preferably between about 218° C. and 222° C., and yet more preferably between about 219° C. and 221° C., e.g., at a temperature of about 220° C.

The medical gases conduit may be configured to have a compliance of less than about 4 ml/cmH₂O, preferably less than about 2.5 ml/cmH₂O, and more preferably less than about 1.2 ml/cmH₂O in at least one conditioned state.

The elongate tube, in isolation from the sheath, may be configured to have a compliance of more than about 4 ml/cmH₂O, preferably more than 5 ml/cmH₂O, in at least one conditioned state.

The medical gases conduit may be configured to have a resistance to flow, in at least one conditioned state, of less than about: 0.06 cmH₂O/l/min at a flow of 30 l/min; 0.12 cmH₂O/l/min at a flow of 15 l/min; or 0.74 cmH₂O/l/min at a flow of 2.5 l/min.

The medical gases conduit may be configured to have an increase in flow resistance with bending of less than 150%, less than 125%, or less than 115%, e.g., about 113%, in at least one conditioned state.

The medical gases conduit configured to comply with any one or more, and preferably all, of the length, leakage, resistance to flow and compliance requirements of the International Organization for Standardization (ISO)'s International Standard 5367: 2014 (E) standard in at least one conditioned state.

The medical gases conduit may be configured to have a compliance of less than about 4 ml/cmH₂O, preferably less than about 2.5 ml/cmH₂O, and more preferably less than about 1.2 ml/cmH₂O after prolonged use.

The elongate tube, in isolation from the sheath, may be configured to have a compliance of more than about 4 ml/cmH₂O, preferably more than 5 ml/cmH₂O after prolonged use.

The medical gases conduit may be configured to have a resistance to flow, after prolonged use, of less than about: 0.06 cmH₂O/l/min at a flow of 30 l/min; 0.12 cmH₂O/l/min at a flow of 15 l/min; or 0.74 cmH₂O/l/min at a flow of 2.5 l/min.

The medical gases conduit may be configured to have an increase in flow resistance with bending of less than 150% after prolong use.

The medical gases conduit may be configured to comply with any one or more, and preferably all, of the length, leakage, resistance to flow and compliance requirements of the International Organization for Standardization (ISO)'s International Standard 5367: 2014 (E) after prolonged use.

The elongate tube and the sheath may be flexible to reduce tube drag forces one or more of a Y-piece and a patient interface of the medical gases system, in use.

The elongate tube, in isolation from the sheath, may be more flexible in a conditioned state than in an equilibrated state.

The medical gases conduit may comprise an expiratory conduit, the medical gases system comprising a respiratory assistance system, and the flow of medical gases comprising a flow of respiratory gases, wherein the expiratory conduit may be configured to form upwards of about 80%, preferably upwards of about 90%, and more preferably upwards of about 95%, of an overall length of an expiratory branch of the respiratory assistance system, in use.

The medical gases conduit or the elongate tube, in an equilibrated state, may have a length of: between about 0.8 m and 1.2 m, e.g., about 1.0 m; between about 1.0 m and 2.5 m, and preferably: between about 1.1 m and 1.4 m, and more preferably between about 1.2 m and 1.3 m, e.g., about 1.25 m; between about 1.4 m and 1.6 m, e.g., about 1.5 m; between about 1.5 m and 1.7 m, e.g., about 1.6 m; or between about 1.5 m and 1.8 m, e.g., about 1.6 m or about 1.8 m; or between about 2.2 m and 2.6 m, more preferably between about 2.3 m and 2.5 m, e.g., about 2.4 m.

The medical gases conduit may measure between about 1.1 m and 1.8 m, preferably between about 1.2 m and 1.8 m, more preferably between about 1.2 m and 1.3 m or between about 1.4 m and 1.6 m or between about 1.5 m and 1.7 m, e.g., about 1.2 m or 1.5 m or 1.6 m, in length, in an equilibrated state.

A wall thickness of the elongate tube, in a dry state, may be between about 0.5 mm and 0.9 mm, preferably between about 0.6 mm and 0.8 mm, more preferably between about 0.65 mm and 0.75 mm, yet more preferably between about 0.68 mm and 0.72 mm, and most preferably between about 0.69 mm and 0.71 mm, e.g., about 0.70 mm.

A wall thickness of the elongate tube, in a saturated state in isolation from the sheath, may be between about 0.7 mm and 1.1 mm, preferably between about 0.8 mm and 1.0 mm, more preferably between about 0.85 mm and 0.95 mm, yet more preferably between about 0.90 mm and 0.94 mm, and most preferably between about 0.91 mm and 0.93 mm, e.g., about 0.92 mm.

A maximum outer diameter of the elongate tube, in a dry state, may be between about 20 mm and 26 mm, preferably between about 21 mm and 25 mm, and more preferably between about 22 mm and 24 mm, e.g., about 23 mm.

A maximum outer diameter of the elongate tube, in a saturated state in isolation from the sheath, may be between about 25 mm and 35 mm, preferably between about 28 mm and 32 mm, and more preferably between about 29 mm and 31 mm, e.g., about 30 mm.

The elongate tube may be extruded.

The elongate tube may be corrugated.

The elongate tube may be corrugated and the sheath may be configured to be at least partially spaced from peaks of corrugations of the elongate tube when the medical gases conduit is in an equilibrated state.

The medical gases conduit may comprise no reinforcing spine or rod within the lumen.

The breathable material may be configured to at least partially dry the flow of medical gases as they pass along a length of the lumen, in use.

The at least one conditioned state may comprise a simulated conditioned state.

The medical gases system may comprise a Y-piece and a gases source, the medical gases conduit configured to be coupled directly to each of: an outlet of the Y-piece; and a gases return inlet of the gases source, or a filter coupled directly to the gases return inlet.

The medical gases conduit may comprise no water trap or provision for a water trap.

The medical gases conduit may be configured for use as or in an expiratory branch of the medical gases system, wherein the expiratory branch does not comprise a water trap.

The medical gases conduit may comprise no heater wire.

Other technical features may be readily apparent to one skilled in the art from the following figures, descriptions, and claims.

In a sixteenth aspect a breathing circuit kit for use in a respiratory assistance system comprises: an inspiratory conduit; a Y-piece; and an expiratory conduit, at least one of the inspiratory conduit and the expiratory conduit comprising the medical gases conduit according to any one of the first to fifteenth aspects.

The breathing circuit kit may comprise a humidification chamber.

The breathing circuit kit may comprise any one or more of: a humidifier supply conduit; a patient interface; a filter; a pressure relief valve; a pressure regulator; and a catheter mount.

The breathing circuit kit may comprise no: heater wire associated with the medical gases conduit; and/or water trap associated with the medical gases conduit.

In a seventeenth aspect, an insufflation circuit kit comprises: a discharge conduit comprising the medical gases conduit of any one of the first to fifteenth aspects; and a discharge filter.

The insufflation circuit kit may comprise any one or more of: an insufflator supply conduit; an adapter; a humidifier supply conduit; an insufflation gas filter; a humidification chamber; a funnel; a delivery conduit; a surgical cannula; a diffuser; and a further discharge conduit.

In an eighteenth aspect, a respiratory assistance system for use in providing respiratory therapy to a patient comprises: a gases source configured to supply a flow of respiratory gases; a humidifier configured to heat and humidify the flow of respiratory gases; a humidifier supply conduit configured convey the flow of respiratory gases from an outlet of the gases source to an inlet of the humidifier; an inspiratory conduit configured to convey the flow of respiratory gases from an outlet of the humidifier to an inlet of a Y-piece or a patient interface for supply to the patient; and an expiratory conduit configured to convey the flow of medical gases from an outlet of the Y-piece or the patient interface to a gases return inlet of the gases source or a filter at the gases return inlet; at least one of the inspiratory conduit and the expiratory conduit comprising the medical gases conduit of any one of the first to fifteenth aspects.

The respiratory assistance system may comprise no: heater wire associated with the medical gases conduit; and/or water trap associated with the medical gases conduit.

In a nineteenth aspect, a surgical insufflation system configured to supply an insufflation gas to a body cavity of a patient comprises: an insufflator configured to supply a flow of insufflation gas; a humidifier configured to heat and humidify the flow of insufflation gas; a humidifier supply conduit configured to convey the flow of insufflation gas from an outlet of the insufflator to an inlet of the humidifier; a delivery conduit configured to convey the flow of insufflation gas from an outlet of the humidifier to an inlet of a surgical cannula for supply to the body cavity of the patient; and a discharge conduit configured to convey the flow of insufflation gas and surgical smoke away from the body cavity of the patient, the discharge conduit comprising the medical gases conduit of any one of the first to fifteenth aspects.

In a twentieth aspect, a method of forming a conduit for use in conveying medical gases comprises: providing a sheath about an elongate tube, the elongate tube defining a lumen for passage of a flow of medical gases; clamping a first end of the sheath to a first end of the elongate tube; and overmolding a connector to the first end of the sheath and the first end of the elongate tube to secure the connector, the sheath and the elongate tube together.

The step of overmolding the connector may comprise: inserting a first connector component at least partially into the lumen of the elongate tube wall at the first end of the elongate tube; and overmolding a second connector component about the first connector component, the first end of the sheath and the first end of the elongate tube.

The method may comprise a step of injection molding the first connector component prior to inserting the first connector component at least partially into the first end of the elongate tube.

The step of clamping the first end of the sheath to the first end of the elongate tube may comprise positioning the first end of the sheath and the first end of the elongate tube within a mold tool, the mold tool comprising a plurality of protrusions engaging and clamping the sheath to the elongate tube.

The plurality of protrusions may form a plurality of apertures in the connector upon overmolding the connector, wherein the sheath is exposed through the plurality of apertures.

The method may comprise steps of: clamping a second end of the sheath to a second end of the elongate tube; and overmolding a second connector to the second end of the sheath and the second end of the elongate tube to secure the second connector, the second end of the sheath and the second end of the elongate tube together.

The method may comprise a step of contracting the sheath in a longitudinal direction so that the second end of the sheath overlies the second end of the elongate tube, prior to clamping the second end of the sheath to the second end of the elongate tube.

The method may comprise a step of extruding the elongate tube.

The method may comprise a step of corrugating the elongate tube.

The method may comprise a step of cutting the elongate tube from a tubular extrusion.

The method may comprise a step of cutting the sheath to a predetermined sheath length from a length of sheathing, wherein the predetermined sheath length is selected so that the sheath is at least partially spaced from the elongate tube in a radial direction when the sheath is provided about the elongate tube and the sheath is deformed to a length of the elongate tube.

The sheath may comprise a tubular mesh, preferably a braided tubular mesh.

The sheath may comprise at least one of: between about 75 and 125 monofilament or multifilament braiding elements, preferably between about 90 and 100 braiding elements, e.g., about 96 braiding elements; and between about 150 and 250 filaments, and preferably between about 180 and 200 filaments, e.g., about 192 filaments. Or the sheath may comprise at least one of: between about 40 and 60 monofilament or multifilament braiding elements, preferably between about 45 and 55 braiding elements, e.g., about 48 braiding elements; and between about 120 and 180 filaments, and preferably between about 135 and 165 filaments, e.g., about 144 filaments.

The elongate tube may comprise a breathable material, preferably a block copolymer, and more preferably a block copolymer comprising: hard segments of polybutylene terephthalate; and soft segments of an ether type macro glycol.

The method may comprise no step of providing at least one, and preferably both, of a heater wire and a water trap to the conduit.

In a twenty-first aspect, a conduit may be formed by the method of the twentieth aspect, wherein the elongate tube may be configured to expand upon absorbing water molecules and the sheath is configured to selectively constrain expansion of the elongate tube, in use.

In a twenty-second aspect a conduit for a respiratory assistance system comprises: an elongate tube defining a lumen for passage of a flow of respiratory gases, the elongate tube comprising a material which is permeable to water molecules and which expands due to absorption of water molecules; and a sheath provided about the elongate tube; wherein the sheath is configured to constrain expansion of the elongate tube due to absorption of water molecules in at least one of a radial direction and a longitudinal direction.

In a twenty-third aspect a conduit for a respiratory assistance system comprises: a corrugated tube defining a lumen for passage of a flow of respiratory gases, the corrugated tube comprising a plurality of corrugations and a material which is permeable to water molecules and expands due to absorption of water molecules; and a sheath provided about the corrugated tube and configured to constrain expansion of the corrugated tube in at least a longitudinal direction of the corrugated tube, in use; wherein the corrugated tube is configured so that a pitch of the plurality of corrugations varies in use, in dependence upon the absorption of water molecules by the corrugated tube and constraint of the corrugated tube by the sheath.

In a twenty-fourth aspect a conduit for a respiratory assistance system comprises: an elongate tube defining a lumen for passage of a flow of respiratory gases, the elongate tube comprising a material which is permeable to water molecules and which expands due to absorption of water molecules; and a sheath provided about the elongate tube, each end of the sheath secured to a corresponding end of the elongate tube, and the sheath is configured so that a length and a diameter of the sheath vary in inverse relation to each other.

In a twenty-fifth aspect a conduit for a respiratory assistance system, comprises: an elongate tube defining a lumen for passage of a flow of respiratory gases; a sheath provided about the elongate tube; and a pair of connectors for pneumatically coupling the elongate tube to other components of the respiratory system, each of the pair of connectors securing a respective end of the sheath to a corresponding end of the elongate tube, and at least a first connector of the pair of connectors comprising a plurality of apertures through which the elongate tube and the sheath are exposed.

In a twenty-sixth aspect a conduit for a respiratory assistance system comprises an elongate tube defining a lumen for passage of a flow of respiratory gases, the elongate tube comprising a material which is permeable to water molecules, wherein the elongate tube is capable of absorbing more than about 33% of its own mass in water molecules in immersion testing, and the conduit does not comprise at least one of a heater wire and a water trap.

In a twenty-seventh aspect a conduit for a respiratory assistance system comprises: an elongate tube defining a lumen for passage of a flow of respiratory gases; and a sheath provided about the elongate tube; wherein the sheath is configured to be at least partially spaced from the elongate tube in a radial direction.

In a twenty-eighth aspect a conduit for use in a respiratory assistance system comprises: an elongate tube defining a lumen for passage of a flow of respiratory gases, the elongate tube comprising a material which is permeable to water molecules and which expands upon absorption of water molecules; and a sheath provided about the elongate tube; wherein a compliance of the conduit in at least some operating conditions is dependent on a physical interaction between the elongate tube and the sheath.

In a twenty-ninth aspect a conduit for use in a respiratory assistance system comprises: an elongate tube defining a lumen for passage of a flow of respiratory gases, the elongate tube comprising a material which is permeable to water molecules; and a pair of connectors at respective ends of the elongate tube for fluidly coupling the conduit with other components of the respiratory assistance system, at least one of the pair of connectors comprising a first connector component inserted at least partially within the lumen at one of the respective ends of the elongate tube, and a second connector component overmolded to the first connector component and the elongate tube.

In a thirtieth aspect, a respiratory assistance system comprises: a gases source configured to generate pressurized breathing gases; a humidifier configured to heat and humidify the pressurized breathing gases; a dryline conduit configured convey the pressurized breathing gases from an outlet of the gases source to an inlet of the humidifier; an inspiratory conduit configured to convey the pressurized breathing gases from an outlet of the humidifier to an inlet of a Y-piece for supply to a patient; and an expiratory conduit configured to convey expired breathing gases from an outlet of the Y-piece to a gases return inlet of the gases source; wherein the expiratory conduit may comprise the conduit of any one of the twenty-first to twenty-ninth aspects.

In a thirty-first aspect a method of forming a conduit for use in a respiratory assistance system comprises: providing a sheath about an elongate tube defining a lumen for passage of a flow of respiratory gases; clamping a first end of the sheath to a first end of the elongate tube; and overmolding a connector to the first end of the sheath and the first end of the elongate tube to secure the connector, the sheath and the elongate tube together.

In a thirty-second aspect a conduit may be formed by the method of the thirty-first aspect, wherein the elongate tube is configured to expand upon absorbing water molecules and the sheath is configured to selectively constrain expansion of the elongate tube.

Other aspects and technical features may be readily apparent to one skilled in the art from the following figures, descriptions, and claims.

### BRIEF DESCRIPTION OF THE DRAWINGS

To easily identify the discussion of any particular element or act, the most significant digit or digits in a reference numeral refer to the figure number in which that element is first introduced. Like reference numerals are used in different drawings and with reference to different examples to refer to like features.

Examples are described in further detail below with reference to the accompanying drawings, in which:

FIG. 1 is a schematic illustration of a respiratory assistance system which may include a conduit in accordance with the present disclosure.

FIG. 2 is a schematic illustration of an alternative respiratory assistance system which may include a conduit in accordance with the present disclosure.

FIG. 3 is a detailed diagram of an example humidifier which may be used in the respiratory assistance system of FIG. 2.

FIG. 4 is a schematic illustration of a bubble CPAP system which may include a conduit in accordance with the present disclosure.

FIG. 5 is a schematic illustration of an alternative bubble CPAP system which may include a conduit in accordance with the present disclosure.

FIG. 6 is a schematic illustration a surgical insufflation system which may include a conduit in accordance with the present disclosure.

FIG. 7 is a schematic illustration of an example conduit in accordance with the present disclosure, in partial-cutaway.

FIG. 8 is a schematic isometric detail illustration of one end of an example conduit in accordance with the present disclosure.

FIG. 9 is a schematic side detail illustration of one end of an example conduit in accordance with the present disclosure.

FIG. 10 is a schematic exploded side detail illustration of one end of an example conduit in accordance with the present disclosure.

FIG. 11 is a schematic cross-sectional side detail illustration of one end of an example conduit in accordance with the present disclosure.

FIG. 12 is a schematic exploded isometric detail illustration of one end of an example conduit in accordance with the present disclosure.

FIG. 13 is a schematic isometric detail illustration of the elongate tube and sheath of an example conduit in accordance with the present disclosure.

FIG. 14 is a schematic side detail illustration of the elongate tube and sheath of an example conduit in accordance with the present disclosure.

FIG. 15 is a schematic end cross-sectional illustration of the elongate tube and sheath of an example conduit in accordance with the present disclosure.

FIG. 16 is a schematic cross-sectional side detail illustration of the elongate tube and sheath of an example conduit in accordance with the present disclosure.

FIG. 17 is a graph of differential scanning calorimetry (DSC) test results for the breathable material of a conduit in accordance with the present disclosure.

FIG. 18 is a schematic detail illustration of the sheath of an example conduit in accordance with the present disclosure, with the conduit in an equilibrated state.

FIG. 19 is a schematic detail illustration of the sheath of FIG. 18, with the conduit in a conditioned state.

FIG. 20 is a graph of the length vs diameter gradients of a selection of braided tubular mesh sheaths which may be used in conduits according to the present disclosure.

FIG. 21 schematically illustrates changes in the state of the elongate tube of an example conduit in accordance with the present disclosure, due to absorption of water molecules and pressurization, without the sheath (in FIG. 21(a)-(d)) and with the sheath (in FIG. 21(e)-(h)).

FIG. 22 is a schematic cross-sectional detail illustration of the profile of a corrugation of an example conduit in accordance with the present disclosure, in an equilibrated state.

FIG. 23 is a schematic cross-sectional detail illustration of the conduit of FIG. 22, in a conditioned state.

FIG. 24 is a schematic cross-sectional detail illustration of a portion of an elongate tube of an example conduit according to the present disclosure, in an equilibrated state.

FIG. 25 is a schematic cross-sectional detail illustration of the elongate tube of FIG. 24, in a conditioned state without a sheath.

FIG. 26 is a schematic cross-sectional detail illustration of the elongate tube of FIG. 24 and FIG. 25, in a conditioned state when constrained by a sheath (not shown).

FIG. 27 is a schematic illustration of a respiratory assistance system including an expiratory conduit in accordance with the present disclosure, in use.

FIG. 28 is a flowchart of an example method of manufacturing a conduit in accordance with the present disclosure.

### DETAILED DESCRIPTION

### System Overview

The present technology relates to medical gases conduits which have particular application in a variety of different medical gases systems, e.g., respiratory assistance systems and surgical insufflation systems, some examples of which are described below.

### Respiratory Assistance System

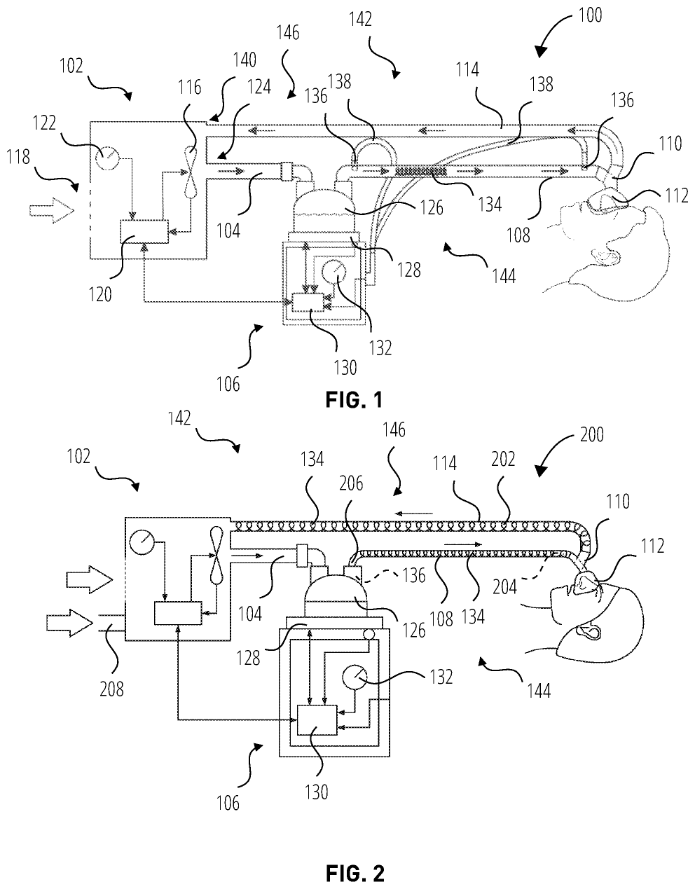

Referring to FIG. 1, a first example respiratory assistance system 100 for providing non-invasive ventilation (NIV) therapy is shown in partial cutaway. The respiratory assistance system 100 has a gases source 102, a humidifier supply conduit 104, a humidifier 106, an inspiratory conduit 108, a Y-piece 110, a patient interface 112 and an expiratory conduit 114.

The gases source 102 in the illustrated example may be a room-entraining ventilator with a pressure generator 116, such as a blower. The pressure generator 116 draws ambient air into the gases source 102 through gas inlet 118 and pressurizes it above ambient pressure to generate a supply of respiratory gases. The ambient air may be supplemented with other gases, such as supplementary oxygen (not shown).

The gases source 102 may have a gases source controller 120 which controls operation of the pressure generator 116. Control may be based on inputs received from one or more of the user interface 122 and one or more sensors (not shown).

In other examples, the gases source 102 may have a positive-displacement pump such as a bellows pump, or may receive respiratory gases from a remote source (i.e., a non-room-entraining ventilator). The gases source controller 120 may control the pressure of the respiratory gases delivered to the patient by controlling a proportional solenoid valve, for example.

Mechanical ventilation can vary from providing supplemental pressure and flow to assist a spontaneously breathing patient (“respiratory support”) to complete control of every breath (“life sustaining”).

For adult patients, respiratory gases may be delivered to the patient and returned to the gases source, e.g., ventilator, at flow rates of up to 120 liters per minute (l/min), e.g., within the range of about 30 to 80 l/min. The respiratory gases may be delivered to the patient and returned to the gases source, e.g., ventilator, at pressures of up to about 6 kilopascal (kPa) (about 60 cmH₂O).

For neonatal or pediatric patients, respiratory gases may be delivered to the patient and returned to the gases source at flow rates of between about 0.5 and 60 l/min. The precise flow rate may depend on the therapy and the weight of the patient. The respiratory gases may be delivered to the patient and returned to the gases source at pressures of up to about 6 kPa (about 60 cmH₂O).

Components of the respiratory assistance system, e.g., the conduits, may be tested and labelled for use to higher pressures, e.g., 8 kPa (about 80 cmH₂O).

Patients receiving mechanical ventilation may be connected to the respiratory assistance system for longer than 24 hours and, depending on the patient's condition, even months or permanently.

In another example, the gases source 102 may be an anesthesia machine. For anesthesia applications, the respiratory assistance system may deliver a mixture of respiratory gases and an anesthetic agent to the patient, e.g., to sedate the patient and render them unconscious for surgery. The anesthetic gas mixture may be delivered to the patient at flow rates of up to about 20 l/min or, for certain “low flow” applications, up to about 10 l/min. The anesthetic gas mixture may be delivered at pressures of up to around 6 kPa. But pressures are typically lower than for mechanical ventilation. The anesthesia machine may include a rebreathing system which delivers gases to a patient via an inspiratory conduit and returns expired gases to the anesthesia machine via an expiratory conduit. The anesthesia machine and breathing circuit generally form a closed loop to prevent the anesthetic agent leaking into the ambient environment. Patients will not normally need to be connected to the anesthesia machine for more than 24 hours. Most patients receiving anesthetic agents would be connected to the breathing circuit for much less than 24 hours. Patients receiving anesthetic agents may be continuously monitored by an anesthetist for the duration of the anesthesia.

The gases source 102 supplies respiratory gases to the humidifier 106 via gases source outlet 124 and humidifier supply conduit 104. In the illustrated example, the humidifier 106 may be an active (heated) passover-type humidifier. A humidification chamber 126 may sit atop a chamber heater 128, or is otherwise in thermal contact with the chamber heater 128. The humidification chamber 126 may be removably engaged with the chamber heater 128. A volume of water within the humidification chamber 126 is heated to vaporize water into the headspace of the humidification chamber 126. The respiratory gases are received from the humidifier supply conduit 104 at an inlet to the humidification chamber 126. The respiratory gases may pass through the headspace of the humidification chamber 126, picking up the water vapor, to an outlet of the humidification chamber 126.

The chamber heater 128 may be controlled by a humidifier controller 130 based on inputs from one or more of a user interface 132 and one or more sensors to achieve a desired level of one or more of heating and humidification of the respiratory gases.

In some examples, the humidification chamber 126 may have a float valve (not shown) to automatically replenish the reservoir of water from a sterile water bag (not shown). The humidifier 106 may be an F&P 810™, 820™, 850™ or 950™ Heated Humidifier available from Fisher & Paykel Healthcare Limited of Auckland, New Zealand, for example. The humidification chamber 126 may include a water feed tube and a water spike (not shown) for fluid coupling with the sterile water bag.

The humidifier controller 130 of the humidifier 106 may control the temperature of the chamber heater 128. The chamber heater 128 may be controlled so that the humidified respiratory gases are received by the patient at, or near, a predetermined temperature and humidity. In the case of a patient receiving invasive ventilation, the patient's upper airway is bypassed and the respiratory gases may be delivered to the patient at a temperature of about 37° C. and about 100% relative humidity. In the case of a patient receiving non-invasive ventilation, the respiratory gases may be delivered to the patient's upper airway at a temperature of about 31° Celsius and 70% relative humidity. The respiratory gases may be further heated and humidified by the patient's upper airway to arrive at the patient's lungs at about 37° C. temperature and about 100% relative humidity.

One or more of the desired temperature and humidity of the respiratory gases may be adjustable using the user interface 132. Alternatively, or additionally, the humidifier 106 and the gases source 102 may be in electronic communication with each other whereby settings of the humidifier 106 may be viewed and/or adjusted from the gases source 102, or vice versa. The humidifier controller 130 may receive signals from one or more sensors to provide open- or closed-loop control of one or more of the temperature and humidity of the respiratory gases.

In other examples, the humidifier 106 may be a passive (unheated) passover-type humidifier, a heat and moisture exchanger (HME), a nebulizing humidifier, or a humidifier supplying a continuous or periodic controlled flow of a humidifying liquid to a heating element for instantaneous or near-instantaneous vaporization.

The respiratory assistance system 100 of FIG. 1 illustrates the gases source 102 and humidifier 106 as separate devices. In other examples, the gases source 102 and humidifier 106 may be integrated with each other. In such examples, the humidifier supply conduit 104 may be, or include, internal ducting within the housing of the integrated device. The functions of the gases source controller 120 and humidifier controller 130 may be performed by a single controller or a distributed control system. Similarly, the user interface 122 and the user interface 132 may be replaced by a single user interface which may receive inputs for control of both the pressure generator 116 and the chamber heater 128.

Referring still to the example respiratory assistance system 100 of FIG. 1, the respiratory gases are conveyed from the humidifier 106 towards the patient by a flexible inspiratory conduit 108. In some examples, the inspiratory conduit 108 may have a length of about 1.0 meters (m) to about 2.5 m. In some examples, the inspiratory conduit 108 may have a length of about 1.5 m to 1.8 m, e.g., about 1.6 m or 1.8 m. In some examples, e.g., for anesthesia applications, the inspiratory conduit 108 may have a length of about 2.2 m to 2.6 m, e.g., about 2.4 m.

The inspiratory conduit 108 may be corrugated. In some examples, e.g., for an adult patient, the corrugated inspiratory conduit 108 may have a maximum outside diameter (that is, the diameter of the inspiratory conduit 108 when measured to exterior surfaces at a peak of a corrugation) of about 20 millimeters (mm) to 30 mm, or about 23 mm to 25 mm, e.g., about 24 mm. In other examples, e.g., for a neonatal or pediatric patient, the maximum outside diameter may be about 10 mm to 20 mm, or about 14 mm to 16 mm, e.g., about 15 mm. The inspiratory conduit 108 may also have a corrugated inner surface. In other examples, the inspiratory conduit 108 may have a substantially smooth (e.g., uncorrugated) inner surface.

The inspiratory conduit 108 may have a heater wire 134 (illustrated only in part, for clarity). The heater wire 134 may be wrapped around an outside of the tube wall, embedded within the tube wall or located in the lumen of the inspiratory conduit 108, for example. The heater wire 134 may be powered by the humidifier 106. The heater wire 134 may be controlled by the humidifier controller 130 to mitigate heat loss of the respiratory gases as they pass along the length of the inspiratory conduit 108. In some examples, the heater wire 134 may maintain, or in some cases increase, the temperature of the respiratory gases as they pass along the length of the inspiratory conduit 108.

In some examples, the inspiratory conduit 108 may have a composite structure made up of two or more components wound around the lumen in a spiral. A first spirally-wound component may be an elongate hollow body, and a second spirally-wound component may be an elongate structural component. The first and second spirally-wound components may be wound in a double-helix configuration. Heating and/or sensing wires may be embedded in the elongate structural component. The inspiratory conduit 108 may have a connector with a printed circuit board (PCB). The heating and/or sensing wires may be connected to the PCB. Further design and manufacturing details of such conduits are disclosed in United States Patent Publication Nos. 2015/0306333, 2017/0100556, 2019/0076620 and 2022/0355059, all assigned to Fisher & Paykel Healthcare Ltd, the entire content of each of which are incorporated herein by reference.

Sensor probes 136 may be removably inserted in apertures at the patient end and the humidifier end of the inspiratory conduit 108. The sensor probes 136 may include one or more of temperature, humidity and flow sensors, for example. Sensor leads 138 may connect the sensor probes 136 to the humidifier 106, so that signals from the sensor probes 136 may be used as inputs in the control algorithms for one or more of the pressure generator 116, chamber heater 128 and heater wire 134. In other examples, the sensors may be integrated in one or more of the humidifier 106 and the inspiratory conduit 108. In some examples, one or more of the sensor leads 138 may be embedded in the tube wall or located in the lumen. In some examples, one or more sensors may be integrated in the inspiratory conduit 108.

The inspiratory conduit 108 may be provided with connectors at opposing ends to establish and maintain respective pneumatic connections with an outlet of the humidification chamber 126 and an inlet of the Y-piece 110. In some examples, e.g., for an adult patient, the connectors may each be an adapter with a 22 mm conical connector. The connectors may have a 1:40 taper complying with the International Organization for Standardization (ISO) 5356-1:2015 (Anaesthetic and respiratory equipment—Conical connectors—Part 1: Cones and sockets) standard. In other examples, e.g., for a neonatal or pediatric patient, the connectors may be any of a 15 mm tapered male conical connector, a 15 mm tapered female conical connector, a 12 mm tapered male conical connector, a 12 mm tapered female conical connector, and a combination of any two thereof.

In some examples, the connector at the humidifier end of the inspiratory conduit 108 may have a socket for receiving the sensor probe 136. In some examples, the connector at the humidifier end of the inspiratory conduit 108 may have a socket for establishing an electrical connection between the humidifier 106 and the heater wire 134. In other examples, the humidifier 106 and the connector may have corresponding integrated electrical contacts whereby both pneumatic and electrical connections may be established in a single action upon physically connecting the inspiratory conduit 108 to the humidifier 106.

The respiratory gases are delivered by the inspiratory conduit 108 to the Y-piece 110 adjacent, or near, the patient. The Y-piece 110 fluidly couples the inspiratory conduit 108 (via the inlet of the Y-piece), the patient interface 112 (via a patient inlet/outlet of the Y-piece) and the expiratory conduit 114 (via an outlet of the Y-piece). It is to be appreciated that while the three limbs (inlet, outlet and patient inlet/outlet) of the Y-piece 110 in this example form a Y-shape, the term is not intended as being limited to this particular configuration.

In some examples, an interface conduit (not shown) may be provided between the patient inlet/outlet of the Y-piece 110 and the patient interface 112.

In other examples, the patient interface 112 may have an integrated Y-piece or separate inlet and outlet for connection of the inspiratory conduit 108 and expiratory conduit 114, respectively. In such examples, the separate Y-piece 110 may be omitted from the system.

The patient interface 112 in the illustrated example is a nasal mask suitable for non-invasive ventilation by supplying the humidified respiratory gases to the patient's nasal passages. In other examples, the patient interface 112 may be any invasive or non-invasive patient interface.

Examples of non-invasive patient interfaces include:
total-face masks, sealing around the patient's eyes, nose and mouth;
full-face masks, sealing around the patient's nose and mouth;
nasal masks, sealing around the patient's nose;
compact nasal masks, sealing with an underside of the patient's nose around the nares;
nasal pillows interfaces, sealing with each of the patient's nares;
sealing nasal cannulae, sealing inside each of the patient's nares;
unsealed nasal cannulae, extending into the patient's nares without occluding the nasal passages;
oral masks, sealing around the patient's mouth;
hybrid masks, combining a compact nasal mask, nasal pillows interface or nasal cannulae with an oral mask; and
combinations of the above (such as nasal cannulae with a full-face mask).

Examples of invasive patient interfaces include:
endotracheal tubes, and
tracheostomy tubes.

Gases expired by the patient, and any excess respiratory gases from the gases source 102 not inspired by the patient, are received by the expiratory conduit 114 from the Y-piece 110. The expiratory conduit 114 conveys the respiratory gases to the gases return inlet 140 of the gases source 102.

The expiratory conduit 114 may have connectors at opposing ends to establish and maintain respective pneumatic connections with an outlet of the Y-piece 110 and a gases source 102 (or optional filter intermediate the expiratory conduit 114 and gases return inlet 140). In some examples, e.g., for an adult patient, the connectors may each be an adapter with a 22 mm conical connector, e.g., a 22 mm tapered female connector. The connectors may have a 1:40 taper complying with the ISO 5356-1:2015 standard. In other examples, e.g., for a neonatal or pediatric patient, the connectors may be any of a 15 mm tapered male connector, 15 mm tapered female conical connector, 12 mm tapered male connector, 12 mm tapered female conical connector, and a combination of any two thereof.

Since the gases inspired and expired by the patient are humidified by the humidifier 106 and, for non-invasive ventilation, the patient's upper airway, the gases received by the expiratory conduit 114 may have a relative humidity as high as 100% (i.e., saturation).

The expiratory conduit 114 does not necessarily have a heater wire. Advantages may be obtained by omitting the heater wire from the expiratory conduit 114, as described in further detail below. But in some examples, the expiratory conduit 114 may be provided with a heater wire. In such examples, the heater wire may be wrapped around the outside of the tube wall, embedded in the tube wall or located in the lumen of the expiratory conduit 114.

The expiratory conduit 114 in this example also does not have, or provide for, a water trap.

The expiratory conduit 114 in some examples may have a length of about 1.0 m to 2.5 m, or about 1.4 m to 1.6 m, e.g., about 1.5 m. In some examples, the expiratory conduit 114 may have a length of about 1.5 m to 1.7 m, e.g., about 1.6 m. In other examples, e.g., for anesthesia applications, the expiratory conduit 114 may have a length of about 2.3 m to 2.5 m, e.g., about 2.4 m. In other examples, the expiratory conduit 114 may have a length of about 1.1 m to 1.4 m, or about 1.2 m to 1.3 m, e.g., about 1.25 m.

The expiratory conduit 114 may be corrugated. In some examples, e.g., for an adult patient, the corrugated expiratory conduit 114 may have a maximum outside diameter (that is, the diameter of the expiratory conduit 114 when measured to exterior surfaces at a peak of a corrugation) of about 20 mm to 30 mm, or about 23 mm to 25 mm, e.g., about 24 mm. In other examples, e.g., for a neonatal or pediatric patient, the corrugated expiratory conduit 114 may have a maximum outside diameter of about 10 mm to 20 mm, or about 14 mm to 16 mm, e.g., about 15 mm. The expiratory conduit 114 may have a corrugated inner surface. In other examples, the expiratory conduit 114 may have a substantially smooth (e.g., uncorrugated) inner surface.

In other examples, the expiratory conduit 114 may be spirally-wound.

In some examples, one or more of the inspiratory conduit 108 and the expiratory conduit 114 may have an identification element. The identification element may be a resistor, a capacitor, or an integrated circuit (IC), for example. The identification element may enable one or more of the humidifier 106 and the gases source 102 to identify the conduit. In some examples, one or more of the humidifier 106 and the gases source 102 may automatically adjust one or more therapy parameters based on identification of the conduit. In some examples, the identification element may enable identification of the type (e.g., model) of the conduit. In some examples, the identification element may communicate with the humidifier 106 or the gases source 102 via a wired connection. In other examples, the identification element may be configured to communicate wirelessly, e.g., using radio frequency identification (RFID).

A filter (not shown) may be provided between the expiratory conduit 114 and the gases return inlet 140. In some examples, a filter may additionally, or alternatively, be provided within the gases source 102.

The humidifier supply conduit 104, humidification chamber 126, inspiratory conduit 108, Y-piece 110 and expiratory conduit 114 together form a breathing circuit. More specifically, this particular configuration forms a dual-limb breathing circuit 142.

The humidifier supply conduit 104, the humidification chamber 126 and the inspiratory conduit 108 form an inspiratory branch 144 of the breathing circuit 142. The inspiratory branch 144 extends from the outlet of the gases source 102 to the Y-piece 110.

The expiratory conduit 114 in this example forms an expiratory branch 146 of the breathing circuit 142. The expiratory branch 146 extends from the Y-piece 110 (or, in some cases, directly from the patient interface 112) to the gases return inlet 140 of the gases source 102 (or, in some cases, the filter at the gases return inlet 140).

The Y-piece 110 may be regarded as part of either, or both of, the inspiratory branch 144 and the expiratory branch 146.

In some examples, as shown in FIG. 1, the expiratory conduit 114 may form the entirety of the expiratory branch 146 (excluding the Y-piece 110). In other examples, the expiratory branch may also include one or more of a filter and a second expiratory conduit connected end-to-end with the expiratory conduit 114. In some examples, the expiratory conduit 114 may form a substantial majority, e.g., upwards of about 80%, upwards of about 90%, or upwards of about 95%, of the length of the expiratory branch 146.

The expiratory conduit 114 may define the entirety of the flow path from the Y-piece 110 (or, in some cases, the patient interface 112) to the gases return inlet 140 of the gases source 102 (or, in some cases, the optional filter). That is, there may be no need for any further conduits in the expiratory branch of the breathing circuit.

With the possible exception of portions of the expiratory conduit 114 inadvertently or temporarily covered by a patient's limbs, clothing, bedding or the like in use, the entirety of the expiratory conduit 114 will generally be exposed to the ambient air of the surrounding environment, e.g., a hospital room.

The technology of the present disclosure is particularly suited for use as the expiratory conduit 114 of the respiratory assistance system 100.

In some examples, the technology of the present disclosure may alternatively, or additionally be suited for use as the inspiratory conduit 108. In an anesthetic procedure, for example, mitigating condensate within the inspiratory conduit 108 may be a higher priority than maintaining 100% relative humidity of the respiratory gases delivered to the patient.

FIG. 2 illustrates a further example respiratory assistance system 200 providing NIV therapy. Like the respiratory assistance system 100, respiratory assistance system 200 has a gases source 102, a humidifier supply conduit 104, a humidifier 106, an inspiratory conduit 108, a Y-piece 110, a patient interface 112 and an expiratory conduit 114. Except as described below or otherwise apparent from the drawings, respiratory assistance system 200 is similar to the respiratory assistance system 100 of FIG. 1 and the description of the respiratory assistance system 100 is intended to apply equally to the respiratory assistance system 200.

In some examples, heater wires 134, 202 may be provided in both the inspiratory conduit 108 and the expiratory conduit 114. But in some examples, at least the heater wire 202 in the expiratory conduit 114 may be omitted.

The heater wire 202 in the expiratory conduit 114 may be wrapped around the outside of the tube wall, embedded within the tube wall or located in the lumen of the expiratory conduit 114, for example.

The expiratory heater wire 202 may be powered by the humidifier 106. The heater wire 202 may be controlled by the humidifier controller 130 to mitigate heat loss of the respiratory gases as they pass along the length of the expiratory conduit 114. In some examples, the heater wire 202 may be controlled to increase, maintain, or in some cases decrease, the temperature of the respiratory gases as they pass along the length of the expiratory conduit 114.

A sensor 204 (e.g., one or more of a temperature, humidity and flow sensor) may be integrated within the inspiratory conduit 108, in a connector of the inspiratory conduit 108. One or more of the sensors 204 may be electrically connected with the humidifier 106 by sensor wires embedded within the tube wall or provided within the lumen of the inspiratory conduit 108.

The inspiratory conduit 108 may have an electropneumatic connector 206 with integrated electrical contacts. The electropneumatic connector 206 may be configured to establish both pneumatic and electrical connections with the humidifier 106 in a single action upon physically connecting the connector to the humidifier 106. This may avoid the need for the removable sensor probes 136 and sensor leads 138 of the respiratory assistance system 100 shown in FIG. 1.

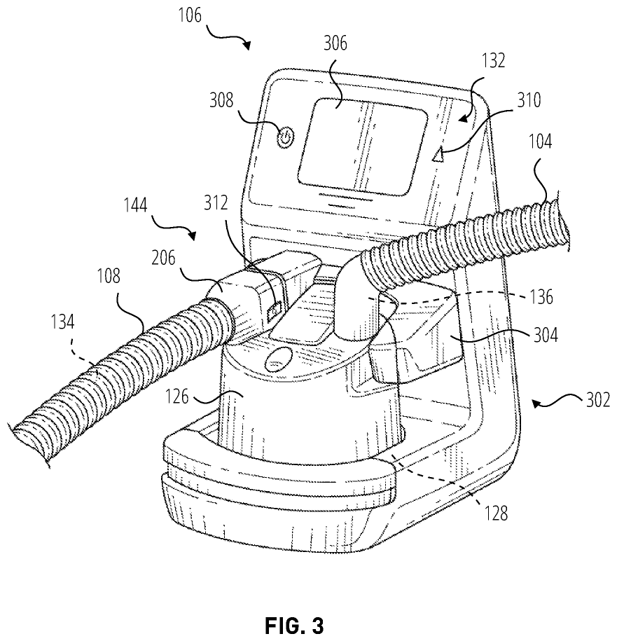

The humidifier 106 may have a sensor probe 136 (hidden in FIG. 2) at or adjacent an outlet of the humidification chamber 126, as described in further detail with respect to FIG. 3.

The gases source 102 in this example has a supplementary gases inlet 208 for provision of supplementary gases, such as supplementary oxygen, to the patient.

The technology of the present disclosure is particularly suited for use as the expiratory conduit 114 of the respiratory assistance system 200. In some examples, e.g., for anesthetic procedures, it may alternatively or additionally be suited for use as the inspiratory conduit 108.

FIG. 3 illustrates in detail an example humidifier 106 which may be used in the respiratory assistance system 200. Also shown (in part) are the humidifier supply conduit 104 and the inspiratory conduit 108.

The humidifier 106 depicted in FIG. 3 is an F&P 950™ respiratory humidifier available from Fisher & Paykel Healthcare. The illustrated humidifier 106 has a heater base 302 and a removable and replaceable humidification chamber 126. The heater base 302 may have a chamber heater 128 (hidden in FIG. 3), a user interface 132 and a controller (not shown). The humidifier 106 may also have a removable and replaceable cartridge 304.

The user interface 132 in this example may include a touchscreen display 306 (i.e., for receiving input from, and providing input to, a user). The user interface 132 may also include one or more of a switch 308 and an indicator light 310. The switch 308 may be a mechanical (e.g., pushbutton or toggle switch) or touch-controlled electrical (e.g., capacitive) switch. The switch 308 may be a power/standby button. The indicator light 310 may be used to indicate when a fault condition occurs. The indicator light 310 may be a light emitting diode (LED). The indicator light 310 may be any suitable shape or form (e.g., a bar, a circle, a rectangle, a square, the perimeter of the touchscreen display 306, etc.). The indicator light 310 may selectively light up in a single color (e.g., red, yellow or green), or in two or more different colors (e.g., yellow and red) to indicate different priority fault conditions.

The humidification chamber 126 may be removably received by the heater base 302, in thermal contact with the chamber heater 128 (hidden beneath the humidification chamber 126 in FIG. 3).

The cartridge 304 may house electronics. The electronics may include one or more sensors. The sensors in the cartridge 304 may sense one or more properties of respiratory gases flowing through the humidification chamber 126 in use. The sensors may be provided on one or more sensor probes 136 (hidden) protruding from the cartridge. The sensor probe 136 may protrude through an aperture in one or more of the inlet and outlet of the humidification chamber, in use. The aperture may be closed by an elastomeric seal. The elastomeric seal may be elastically deformed by the sensor probe 136 when the humidification chamber 126 is received by the heater base 302.

The cartridge 304 may have an electrical connector which makes an electrical connection with the heater base 302 for communication (e.g., serial communication) with the controller. The cartridge 304 may have a microcontroller communicatively coupled with the sensor(s) and the controller.

In use, the outlet end of the humidifier supply conduit 104 is pneumatically coupled with the inlet of the humidification chamber 126. The electropneumatic connector 206 of the inspiratory conduit 108 is electrically coupled with the cartridge 304 and pneumatically coupled with the outlet of the humidification chamber 126 to transport the humidified flow of gases towards the patient. The electropneumatic connector 206 may make a releasable and lockable connection with one or more of the humidification chamber 126 and the cartridge 304. The electropneumatic connector 206 may have a release button 312. The release button 312 may be actuated to facilitate disconnection of the electropneumatic connector 206 from the humidifier 106.

The electropneumatic connector 206 may be configured so that it can be physically and pneumatically coupled with the humidification chamber 126 before the humidification chamber 126 is installed on the heater base 302. The electropneumatic connector 206 may be configured so that it can make an electrical connection with the humidifier 106 as the humidification chamber 126 is installed on the heater base 302 in a sliding motion (e.g., in a horizontal direction).

Alternatively, or additionally, the electropneumatic connector 206 may be configured to that it can be physically and pneumatically coupled with the humidification chamber 126 when it is already installed on the heater base 302, and physically and electrically connected with the humidifier 106 substantially simultaneously, e.g., in a single action.

The inspiratory conduit 108 in one example may have a substantially smooth inner surface. The inspiratory conduit 108 may have a lower resistance to flow than an equivalent corrugated tube, and may accordingly have a relatively smaller inside diameter (when compared to the minimum inside diameter of a corrugated tube). For example, the inspiratory conduit 108 in this example may have a substantially uniform inside diameter of about 15 mm to 19 mm, e.g., about 17 mm, for adult patients or about 10 mm to 14 mm, e.g., about 12 mm, for neonatal or pediatric patients. In some examples, the inspiratory conduit 108 may be spirally-wound.

The electropneumatic connector 206 may have electrical terminals respectively coupled with at least one of a pair of sensor wires and a pair of heater wires 134 (hidden in FIG. 3). One or more of the sensor wires and the heater wires 134 may be embedded within the tube wall of the inspiratory conduit 108. The sensor wires and heater wires 134 may form respective sensing and heating circuits.

The electropneumatic connector 206 may have electrical terminals electrically coupled with an identification element, e.g., a resistor, embedded within the electropneumatic connector 206. The controller of the humidifier 106 may be configured to identify the type of inspiratory conduit 108 coupled with the humidifier 106, e.g., by applying a voltage across the respective electrical terminals and measuring a current through the resistor. A resistance of, e.g., about 100 Ohms (52) may indicate to the controller that a first type of inspiratory conduit 108, e.g., an adult conduit, is connected to the humidifier 106, and a resistance of, e.g., about 200 $2 may indicate to the controller that a second type of inspiratory conduit 108, e.g., a neonatal conduit, is connected to the humidifier 106.

In other examples, the electropneumatic connector 206 may have an identification element comprising an IC with a non-transitory memory configured to store identification data, as described above.

The electrical terminals of the electropneumatic connector 206 may be configured to make an electrical connection with corresponding terminals on the cartridge 304. Thus, existing heater bases 302 may be retrofitted with a replacement cartridge 304 which may include one or more of additional electronics and electrical terminals to provide new functionality to the humidifier 106. Similarly, the humidifier 106 may be retrofitted with a replacement cartridge 304 for compatibility with alternative inspiratory conduits, if necessary. Alternatively, the electrical terminals of the electropneumatic connector 206 and cartridges 304 may be arranged so that selected “core” terminals make electrical connections with corresponding terminals of two or more different cartridges, while “optional” terminals or pads make electrical connections only with specific terminals or pads of selected cartridges configured to make use of those connections.

At a distal end (i.e., patient end, not shown in FIG. 3) of the inspiratory conduit 108, a sensor, e.g., a temperature sensor, may be electrically coupled to the pair of sensing wires, forming a sensing circuit. The heating wires may be electrically coupled with each other, forming a heating circuit. Additional wire(s) or conductor(s) may similarly be electrically coupled at the distal end of the tube, if required.

The humidifier 106, e.g., cartridge 304 may have a socket or integrated cable configured for connection to the expiratory conduit 114, e.g., to supply power to the optional expiratory heater wire 202 (not shown in FIG. 3).

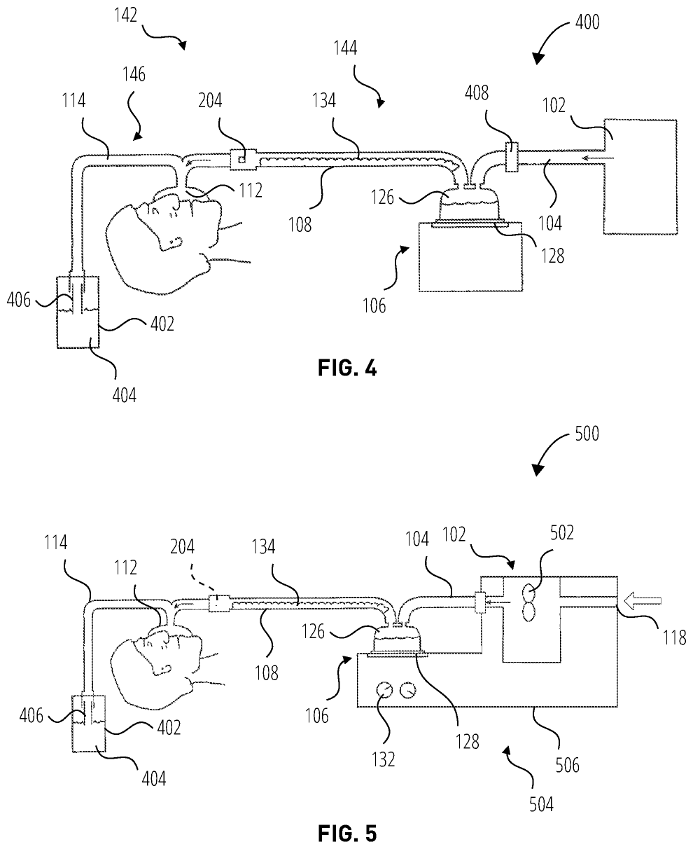

Referring to FIG. 4, a further example respiratory assistance system 400 is shown, this time in the form of a bubble CPAP system providing bubble CPAP (continuous positive airway pressure) therapy.

Except as described below and shown in the drawings, the respiratory assistance system 400 of FIG. 4 may be similar to the respiratory assistance systems 100, 200 of FIG. 1 and FIG. 2, respectively.

The gases supplied to the patient may be ambient air, oxygen, a mixture of the two, or a mixture of ambient air and other auxiliary gas(es). The gases may include medicaments. The medicaments may be added through nebulization. In this example, the gases source 102 may be, or include, a wall source, e.g., in a hospital. The flow of gases delivered to the patient in the respiratory assistance system 400 may be delivered at a substantially constant flow rate.

For neonatal or pediatric patients, respiratory gases in a bubble CPAP system may be delivered to the patient at a flow rate of about 2 liters per kilogram per minute (L/kg/min). In some examples, the flow rate may be between about 1 l/min and 15 l/min, between about 4 l/min and 15 l/min, or between about 6 l/min to 8 l/min. The respiratory gases may be delivered to the patient at pressures of between about 3 cmH₂O and 10 cmH₂O.

The respiratory assistance system 400 may have a pressure regulator 402. In the illustrated example, the pressure regulator 402 is in the form of a bubbler.

The bubbler may be configured to contain a volume of liquid 404, e.g., water. The bubbler may have an adjustable inlet probe 406 configured to extend into the bubbler for submersion in the liquid 404. The inlet probe 406 may be configured to be pneumatically coupled with the expiratory conduit 114. The bubbler can act as a pressure regulator by venting gases from the respiratory assistance system 400 when the pressure exceeds a desired level. This may maintain the average or mean pressure at the target level. The target pressure level can be adjusted by submerging the inlet probe 406 to a desired depth within the liquid 404.

A potential problem with condensate in the breathing circuit of a bubble CPAP system is that at least some of the condensate may drain into the bubbler. This may increase the volume of the liquid 404 in the bubbler, increasing the depth of the inlet probe 406. This, in turn, may increase the positive end expiratory pressure (PEEP) above the target level, in use.

During use, gases escape from a terminal end of the inlet probe 406 and bubble to the surface of the liquid 404. This bubbling may cause small fluctuations in the pressure of the gases. These fluctuations may have therapeutic benefits for the patient.

The respiratory assistance system 400 may have a pressure relief valve 408 for venting excess gases when the pressure exceeds a selected level.

The example respiratory assistance system 400 provides another example of a dual-limb breathing circuit 142. The term “circuit” is not intended to require that the breathing circuit must necessarily form a closed loop, although it may do so in at least some examples. The inspiratory branch 144 may also include the optional pressure relief valve 408. The expiratory branch 146 may also include the pressure regulator 402.

Referring to FIG. 5, a further example of a respiratory assistance system 500 is shown, again in the form of a bubble CPAP system. Except as described below, the respiratory assistance system 500 of FIG. 5 may be similar to the respiratory assistance system 400 of FIG. 4.

In this example, the gases source 102 includes a flow generator, e.g., a blower 502. The blower 502 may include an electric motor which drives an impeller to pressurize ambient air to generate a supply of respiratory gases. Using a flow generator to generate the flow of gases can allow the respiratory assistance system 500 to provide bubble CPAP therapy without the need for a wall source. The gases source 102 may include a battery power source for the blower 502. The battery power source may allow the respiratory assistance system 500 to provide therapy, at least temporarily, without connection of the gases source 102 to a power outlet with a power cable, e.g., while a patient is moved between hospital wards.

The flow generator may draw in ambient air for supply to the patient. The respiratory assistance system 500 may be simpler and cheaper to use as there is no requirement for a gas store or a gas source, e.g., a wall source. Use of a flow generator to pressurize abundant ambient air may eliminate the risk of exhausting a supply of gases. Exhausting the supply of gases may interrupt therapy to the patient.

The gases source 102 and the humidifier 106 may be integrated as a single respiratory therapy device 504. In some examples, the gases source 102 and the humidifier 106 may share a single housing 506. The humidification chamber 126 may be removable from the housing 506 for replacement or refilling. In other examples, the housing 506 may be formed by a separate flow generator housing and humidifier housing which may be attached together to form an integrated respiratory therapy device 504.

The humidifier supply conduit 104 may be, or include, ducting within the housing 506.

The respiratory therapy device 504 may include a gas inlet 118. The gas inlet 118 may receive ambient air from the environment surrounding the respiratory therapy device 504. The gas inlet 118 may include a filter to filter the ambient air.

The respiratory therapy device 504 may have a supplementary gases inlet (not shown) for receiving a supplementary gas, e.g., oxygen, or gases to be supplied to the patient. The respiratory therapy device 504 may have a gases blender to mix together the ambient air and the supplementary gas. The controller may be configured to control the concentration of the supplementary gas supplied to the patient, e.g., using a proportional valve (not shown).

The gases source 102 and the humidifier 106 may share a single controller (not shown) controlling operation of any one or more of the chamber heater 128, the heater wire 134, the blower 502 and the proportional valve.

In some examples, the expiratory conduit 114 does not have a heater wire. In other examples, the expiratory conduit 114 may have a heater wire. In some examples, the expiratory conduit 114 does not have a water trap. In some examples, the expiratory conduit 114 does not have either of a heater wire and a water trap.

The technology of the present disclosure is particularly suited for use in the expiratory conduit 114 of the respiratory assistance system 400.

One or more of the inspiratory conduit 108 and the expiratory conduit 114 of each of the example respiratory assistance systems 100, 200, 400, 500 (and others) may be sold individually or packaged and sold as part of a breathing circuit kit. The breathing circuit kit may include combinations of any two or more of the:
humidifier supply conduit 104;
humidification chamber 126;
inspiratory conduit 108;
Y-piece 110;
patient interface 112;
catheter mount;
expiratory conduit 114;
filter;
pressure relief valve 408;
pressure regulator 402; and
other accessories such as a pressure line and one or more connector adapters.

In some examples, the breathing circuit kit may include the inspiratory conduit 108, Y-piece 110 and expiratory conduit 114. The breathing circuit kit may optionally include one or more of the other components listed above, e.g., the humidification chamber 126.

In some examples, the breathing circuit kit may include the humidifier supply conduit 104, inspiratory conduit 108, Y-piece 110, expiratory conduit 114 and filter. The breathing circuit kit may also include the humidification chamber 126. The breathing circuit kit may optionally include one or more of the other components listed above.

In other examples, the breathing circuit kit may include the humidifier supply conduit 104, inspiratory conduit 108, expiratory conduit 114, and pressure regulator 402. The pressure regulator 402 may be a bubbler. The breathing circuit kit may also optionally include one or more of the humidification chamber 126 and the pressure relief valve 408. The breathing circuit kit may optionally include one or more of the other components listed above.

In some examples, the breathing circuit kit may be partially, or wholly, pre-assembled. Preassembly of the breathing circuit may facilitate quicker setup of the breathing circuit. Preassembly may reduce the risk of misconnections, e.g., transposition of the inspiratory conduit 108 and expiratory conduit 114.

In some examples, the breathing circuit kit may be packaged together, e.g., in a sealed plastic bag. A number of breathing circuit kits, e.g., 10 breathing circuit kits, may be packaged together, e.g., in a cardboard box.

### Surgical Insufflation System

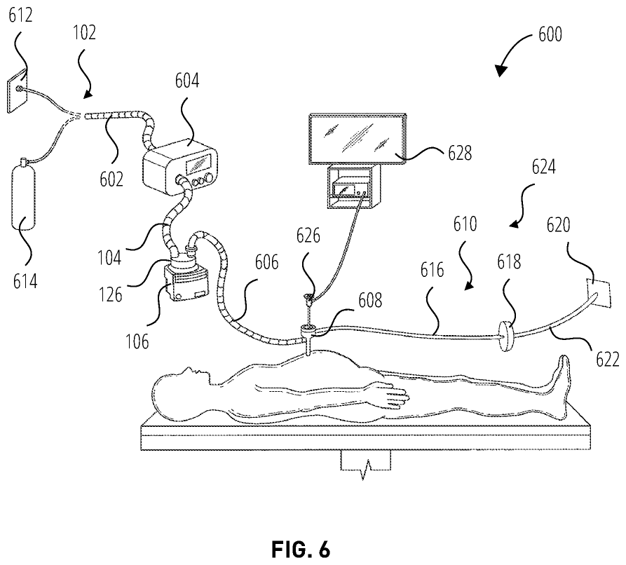

Referring to FIG. 6, a surgical insufflation system 600 is shown. The surgical insufflation system 600 may be configured to supply an insufflation gas to a body cavity of a patient, e.g., for laparoscopic procedures.

In some examples, the insufflation gas may be carbon dioxide. In some examples, a medicament may be added to the insufflation gas.

The insufflation gas is pressurized to a pressure above atmospheric pressure. The insufflation gas may create a working space within the body of the patient, e.g., the abdominal or peritoneal cavity, for a surgeon or surgical team to carry out a surgical procedure. The surgical procedure may involve cauterization creating surgical smoke in the working space.

The surgical insufflation system 600 may include one or more of a gases source 102, an insufflator supply conduit 602, an insufflator 604, an insufflation gas filter (not shown), a humidifier supply conduit 104, a humidifier 106, a delivery conduit 606, a surgical cannula 608 and a smoke evacuation system 610. In other examples, the surgical insufflation system may include a recirculation system (not shown), e.g., in place of the smoke evacuation system 610.

The gases source 102 may be a wall source 612 or a compressed gas cylinder 614, for example. In other examples, the insufflator may be configured to pressurize ambient air.

One or more insufflation gas filters may be provided between the insufflator 604 and the surgical cannula 608. An insufflation gas filter may be provided between the insufflator 604 and the humidifier 106, e.g., between an outlet of the insufflator 604 and an inlet end of the humidifier supply conduit. An insufflation gas filter may alternatively, or additionally, be provided between the humidifier 106 and the surgical cannula 608, e.g., between an outlet of the humidifier 106 and an inlet end of the delivery conduit 606.

An adapter (not shown) may be provided at the outlet of the insufflator 604, e.g., between the insufflator 604 and the insufflation gas filter or humidifier supply conduit 104.

In other examples, e.g., a surgical insufflation system configured for open surgery, the insufflation gas filter or humidifier supply conduit 104 may be connected directly to a CO₂ gas supply stand gas outlet port.

The humidifier 106 may include one or more of a humidification chamber, a chamber heater, a humidifier controller and a user interface as described above with respect to the example respiratory assistance systems 100, 200, 400, 500. The humidifier 106 in this example may be an F&P HumiGard™ SH870 Surgical Humidifier, also available from Fisher & Paykel Healthcare. In other example surgical insufflation systems, the humidifier 106 may be omitted or may be disabled for the entire procedure or part of the procedure.

A funnel may be provided to assist in filling the humidification chamber 126 with a liquid, e.g., sterile water, through the inlet or outlet of the humidification chamber 126.

A connector at one end of the delivery conduit 606 may be a luer lock connector. The luer lock connector may be configured to connect directly to the surgical cannula 608. In other examples, e.g., a surgical insufflation system configured for open surgery, the delivery conduit 606 may instead be configured to be connected to a diffuser.

The delivery conduit 606 may otherwise be similar to the inspiratory conduit 108 of the respiratory assistance systems 100, 200, 400, 500. For example, the delivery conduit 606 may have a heater, e.g., heater wire 134. In some examples, the delivery conduit 606 may be corrugated. In other examples, the delivery conduit may be spirally-wound.

The smoke evacuation system 610 may include a discharge conduit 616 and a discharge filter 618.

In some examples, the discharge conduit 616 may be between about 0.8 m and 1.2 m, e.g., about 1.0 m, in length.

The smoke evacuation system 610 may be configured to be coupled with a vacuum source 620, e.g., a wall source. In some examples, the smoke evacuation system 610 may include a further discharge conduit 622. The further discharge conduit 622 may be configured to be coupled between the discharge filter 618 and the vacuum source 620. In some examples, the further discharge conduit 622 may have generally the same construction as the discharge conduit 616. In some examples, the further discharge conduit 622 may have the same length as the discharge conduit 616. In other examples, the further discharge conduit 622 may have a different construction to the discharge conduit 616. In some examples, the further discharge conduit 622 may differ from the discharge conduit 616 in one or more of length, diameter and materials.

The recirculation system (not shown) may include the discharge conduit 616, the discharge filter 618 and a further discharge conduit that is coupled to a further surgical cannula. This may enable exhausted insufflation gas to be filtered and then recirculated back into the body cavity of the patient.

In the illustrated example surgical insufflation system 600, the humidifier supply conduit 104, humidification chamber 126, delivery conduit 606, discharge conduit 616, discharge filter 618 and further discharge conduit 622 form an insufflation circuit. More specifically, this particular configuration forms a dual-limb insufflation circuit 624. The humidifier supply conduit 104, humidification chamber 126 and delivery conduit 606 may be said to form an inlet branch of the dual-limb insufflation circuit 624. The smoke evacuation system 610 (e.g., discharge conduit 616, discharge filter 618 and further discharge conduit 622) may be said to form an outlet branch of the dual-limb insufflation circuit 624.

Also shown in FIG. 6 are a scope 626 and a laparoscopic monitor 628.

In use, insufflation gas is supplied by the gases source 102, through the insufflator supply conduit 602, to the insufflator 604. The insufflator 604 may control the pressure of the insufflation gas. The insufflator 604 supplies the insufflation gas, through the humidifier supply conduit 104, to an inlet of the humidifier 106. The insufflation gas is heated and humidified by the humidifier 106. The heated and humidified insufflation gas is then supplied, through the delivery conduit 606, to the surgical cannula 608 and then into the patient's body cavity.

In some examples, the insufflator 604 pressure may be set between about 5 mm/Hg and 20 mm/Hg, depending on the size of the patient and the amount of inflation required. The flow rate of the insufflator 604 may be set to between about 1 L/min and 12 L/min, depending on the requirements of the specific operation.

The insufflation gas and surgical smoke, if any, may flow from the patient's body cavity into the surgical cannula 608 and through the discharge conduit 616 to the discharge filter 618. In some examples, the insufflation gas and surgical smoke, if any, may flow from the patient's body cavity into a dedicated venting cannula (not shown) and through the discharge conduit 616 to the discharge filter 618. That is, the surgical cannula 608 is not necessarily connected to the discharge conduit 616.

The discharge filter 618 may include a filter medium. The filter medium may trap contaminant material in the insufflation gas or the surgical smoke. The contaminant material may include one or more of particulate matter, odors and gaseous hydrocarbons. In some examples, the filtered gas downstream from the discharge filter 618 may be almost 100% carbon dioxide. In some examples, the discharge filter removes 99.999% of all particles, cells and viruses. In some examples, the discharge filter may have retention up to 0.02 microns. The filtered gas may be vented to ambient air. It may be vented remotely from the patient and surgical team.

In some examples, the humidifier 106 may be configured to humidify the insufflation gases to, or near, saturation, i.e., 100% relative humidity. As the patient's body cavity is already moist and humid, the insufflation gas may not lose much, if any, moisture in the body, and can become fully saturated if it is not already at saturation point.

In other examples, a surgical insufflation system may omit the humidifier 106, and may supply relatively dry insufflation gas to the patient, e.g., at a relative humidity of below about 90%, below about 80%, or below about 70%. The relatively dry insufflation gas may be humidified as it passes through the patient's body cavity, picking up moisture from the relatively warm and humid environment in the body cavity above the internal organs.

When the insufflation gas passes out of the patient's body cavity, it passes along the tube wall of the discharge conduit 616. The tube wall, being exposed to ambient air, may be cooler than the insufflation gas. The water vapor in the insufflation gas may condense out of the gas onto the tube wall of the discharge conduit 616. The water vapor can also condense on, or in, one or more other components of the insufflation circuit, e.g., the discharge filter 618 and the further discharge conduit 622.

One or more of water vapor condensing in the discharge filter 618 and run-off from the discharge conduit 616 can saturate the discharge filter 618. The discharge filter 618 may become at least partially occluded. Occlusion of the discharge filter 618 may cause an increase in back-pressure. Occlusion of the discharge filter 618 may hinder dissipation of surgical smoke within the patient's body cavity. Surgical smoke lingering in the patient's body cavity or discharge conduit 616 may be hazardous to the patient. The vision of the surgeon can be obstructed or hindered due to the lingering surgical smoke. Impeded filtration may result in potentially harmful contaminant material escaping into the operating theatre.

The technology of the present disclosure is particularly suited for use in the smoke evacuation system 610 of the surgical insufflation system 600, e.g., as one or more of the discharge conduit 616 and the further discharge conduit 622. In some examples, the smoke evacuation system 610 does not have one or more of a heater wire and a water trap. But in some examples, one or more of the discharge conduit 616 and the further discharge conduit 622 may have a heater wire.

The smoke evacuation system 610 of the example surgical insufflation system 600 may be sold individually or as part of an insufflation circuit kit. The insufflation circuit kit may include the discharge conduit 616 and one or more of the:
insufflator supply conduit 602;
adapter;
insufflation gas filter;
humidifier supply conduit 104;
humidification chamber 126;
funnel;
delivery conduit 606;
surgical cannula 608;
diffuser;
discharge filter 618;
further discharge conduit 622.

In one example, the insufflation circuit kit may include the discharge conduit 616 and the discharge filter 618.

In another example, the insufflation circuit kit may include the adapter, insufflation gas filter, humidifier supply conduit 104, humidification chamber 126, funnel, delivery conduit 606, discharge conduit 616 and discharge filter 618.

In another example, the insufflation circuit kit may include the insufflation gas filter, humidifier supply conduit 104, humidification chamber 126, delivery conduit 606 and discharge conduit 616 and discharge filter 618.

In some examples, the insufflation circuit kit, or one or more components of the kit, may be packaged, e.g., in a sealed plastic bag. A number of insufflation circuit kits, e.g., 10 insufflation circuit kits, may be packaged together, e.g., in a cardboard box.

In some examples, the insufflation circuit kit may be partially, or wholly, pre-assembled. Preassembly may facilitate quicker setup of the insufflation circuit. Preassembly may reduce the risk of misconnections.

In one example, the insufflation gas filter, humidifier supply conduit 104 and humidification chamber 126 may be preassembled with each other. The delivery conduit 606 in this example is not preassembled with any other components of the insufflation circuit. The insufflation gas filter, humidifier supply conduit 104 and humidification chamber 126 may be non-sterile. The delivery conduit 606 may be sterile. The delivery conduit 606 may be packaged individually, e.g., within a sealed plastic bag. The individually packaged delivery conduit 606 may be packaged along with the insufflation gas filter, humidifier supply conduit 104 and humidification chamber 126, e.g., in another plastic bag.

### Conduit Structure

An example conduit according to the present disclosure will be described below with reference to a conduit 700 suitable for use as the expiratory conduit 114 in the example respiratory assistance systems 100, 200, 400, 500. But the present technology may be suitable for application as any one of the other conduits in the respiratory assistance systems 100, 200, 400, 500, in alternative respiratory assistance systems, or in other medical systems. For example, conduits according to the present disclosure may be used as the inspiratory conduit 108 in a breathing circuit configured for use with an anesthesia machine, or as the discharge conduit 616 in the surgical insufflation system 600, for example.

Accumulation of condensate in an inspiratory conduit of a respiratory assistance system may be reduced by lowering the level of active humidification, in turn lowering the relative humidity of the gases delivered to the patient. But this is not optimal for patient comfort or recovery. Nor will it necessarily address condensate in the expiratory conduit due to further humidification of the gases in the patient's upper airway during NIV therapy. Nor will it necessarily address condensate draining into one or more of the inspiratory branch and the expiratory branch from interstitial patient interface components, such as a catheter mount.

Other options to mitigate condensate in the inspiratory conduit and the expiratory conduit include the use of one or more of thermal insulation, a water trap and a heater.

Insulating the conduit reduces the rate of heat loss of the respiratory gases as they pass along the length of the conduit. The conduit may be provided with an outer jacket of thermally insulative material or an air gap in the tube wall, for example. However, such insulation may increase one or more of the diameter, weight and cost of the conduit; impair flexibility of the conduit; or have limited effectiveness. Nor does insulation address condensate which may drain into the conduit from another source, e.g., any one or more of the gases source, the filter, the Y-piece, the catheter mount and the patient interface.

A water trap is intended to collect accumulated condensate for disposal. The inspiratory conduit and the expiratory conduit are each often draped between the patient and a ventilator, and condensate may accumulate at the lowest point of the conduit. The water trap is generally provided at or near the middle of the expiratory branch so that it is close to the anticipated lowest point of the expiratory branch, in use. But the position of the water trap is fixed and may not necessarily coincide with the lowest point. A medical professional may need to periodically manipulate the conduit by elevating portions it so that accumulated condensate drains towards, and into, the water trap. If not done carefully, this can cause some condensate to simultaneously drain towards the patient or the ventilator, for example. And condensate may become trapped within corrugations of the conduit. Moreover, the added weight of the water trap and collected condensate may increase tube drag forces. And the water trap requires periodic emptying. This may interrupt therapy to the patient. Emptying the water trap also presents an infection risk. A water trap goes some way towards addressing the problem of condensate once it is present. And it may also go some way towards addressing condensate from other sources. But it does not address the cause of the problem, and comes with its own disadvantages.

In some examples, conduits according to the present disclosure may omit a water trap.

A heater is intended to maintain or increase the temperature of the respiratory gases above their dew point. This may mitigate formation of condensate within the conduit. The heater may include a resistive heater wire provided within the lumen of the conduit or embedded within the tube wall of the conduit. Potential downsides of a heater wire may include one or more of:
The need for establishing electrical connections between the conduit and a power source (such as a humidifier of the respiratory assistance system);
Added complexity in the heating and humidification algorithms;
Relatively high temperature and absolute/relative humidity of the gases received by the ventilator;
The need to comply with additional standards specific to heated-wire breathing tubes (e.g., International Electrotechnical Commission (IEC) 60601-1 (IEC: 2005+A1:2012(E)), Section 11.2);
Reduced shelf life; and
Power consumption during use of a heated conduit contributes to the overall carbon footprint, e.g., about 50% of the carbon footprint, of the heated conduit over its lifetime.

An alternative form of heater is a “water jacket” heater, whereby heated water (or other liquid) is circulated around the outside of the lumen to heat the flow of respiratory gases within the lumen. This has the further disadvantages of increased bulk, weight and cost of the conduit, the additional need for heating and pumping of the water, and the risk of water leaking into the lumen of the conduit.

In some examples, conduits according to the present disclosure may omit a heater, e.g., both a heater wire and a water jacket. In other examples, the conduits may include a heater wire. But omission of a heater wire may have one or more of the advantages of:
simplifying manufacture;
reducing manufacturing and materials costs;
improving usability by avoiding the need for establishing an electrical connection between the conduit and a power source such as the humidifier 106 or gases source 102;
avoiding the need for heater wire control algorithms;
improving safety;
reducing surface temperatures;
improving pneumatic performance;
reducing regulatory burdens (e.g., International Electrotechnical Commission (IEC) 60601-1 (IEC: 2005+A1:2012(E)), Section 11.2);
reducing the temperature of gases received at the gases return inlet 140;
improved reliability;
longer shelf life (e.g., where electrical insulation is a limiting factor);
longer duration of use (e.g., where electrical insulation is a limiting factor); and
reducing the power consumption, and thus the carbon footprint, of the conduit.

Another approach to mitigating condensation within a conduit, particularly an expiratory conduit, is to form the tube wall from a breathable material (defined in the Glossary below). The breathable material is permeable to water molecules, yet relatively impermeable to liquid water and respiratory gases. Water molecules within the lumen of such conduits may be absorbed by the breathable material of the tube wall, diffused through the breathable material, and desorbed to ambient air according to a gradient moving from the higher humidity side to the lower humidity side. This is known as the solution-diffusion mechanism. By contrast, porous membranes such as expanded PTFE (ePTFE) fabrics (e.g., Gore-Tex™ fabric available from W. L. Gore & Associates) are permeable to water molecules (and, potentially, pathogens) by the pore-flow mechanism, whereby water passes through pores (i.e., holes) extending from one side of the membrane to the other.

Under a scanning electron microscope (SEM) at a magnification of 150× or 2500×, an unfoamed breathable material may be devoid of any channels or pores. A foamed breathable material may have a number of closed-cell voids, but no open channels or pores extending from one major surface to the other. A porous membrane will have open channels or pores extending from one major surface to the other.

Use of a breathable material may reduce the absolute humidity of the flow of respiratory gases within the lumen, and thus the dew point of the respiratory gases, as they pass along the length of the conduit. Examples of such conduits are the expiratory conduits of various breathing circuits with EVAQUA™ technology available from Fisher & Paykel Healthcare, such as the RT280™ and RT340™ Adult Breathing Circuits with EVAQUA™ Technology. These expiratory conduits omit the water trap, but do include a heater wire. They have proven effective in reducing the formation of condensate within the conduit. But it has been found that in at least some conditions, condensate, e.g., from other sources (such as the catheter mount, patient contribution, nebulizer or the like), may still accumulate in the conduit during prolonged use.

Details of such breathable conduits are disclosed in U.S. Pat. Nos. 6,769,431 and 10,532,177, both assigned to Fisher & Paykel Healthcare Limited.

It has been found that breathable materials with relatively higher permeability to water molecules may be more effective in mitigating condensate. But such breathable materials may absorb a relatively large mass of water molecules in use, which has been found to significantly alter the mechanical properties of the conduit.

This presents challenges in meeting certain standards or other design requirements. Respiratory breathing circuit conduits may be required to comply with certain minimum requirements specified by formal standards such as the ISO 5367:2014(E) (Anaesthetic and respiratory equipment—Breathing sets and connectors) standard. This standard specifies basic requirements, such as:
i. materials,
ii. length,
iii. connections,
iv. leakage,
v. resistance to flow, and
vi. compliance.

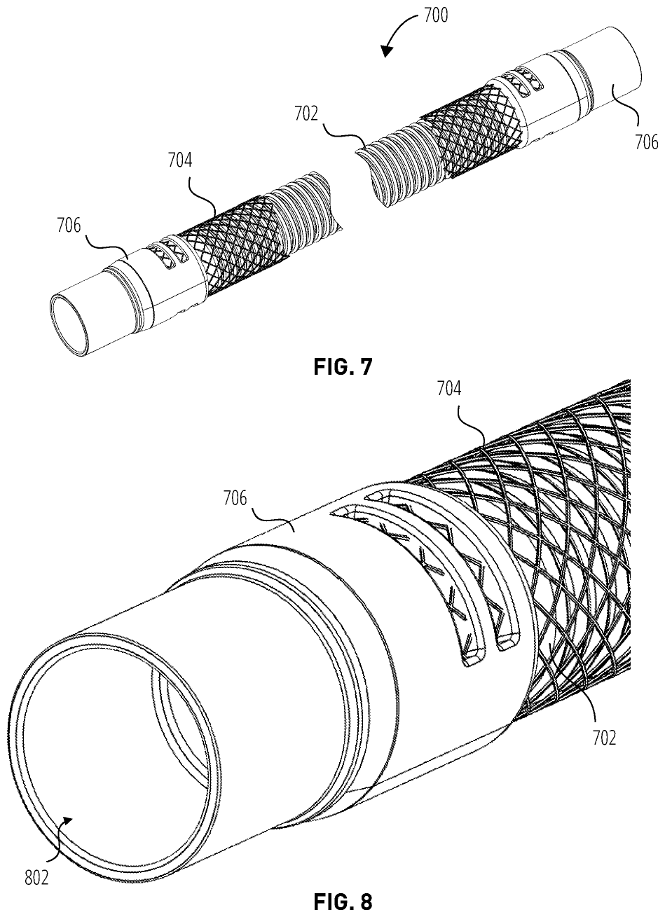

FIG. 7 shows an isometric schematic view of example conduit 700 according to the present disclosure.

The conduit 700 may be used in a respiratory assistance system such as the respiratory assistance systems 100, 200, 400, 500 illustrated by way of example in FIG. 1, FIG. 2, FIG. 4 and FIG. 5, respectively. In particular, the conduit 700 may be used as the expiratory conduit 114 in the respiratory assistance system 100 and will be described in this context below.

As shown, the conduit 700 has an elongate tube 702, a sheath 704 provided about the elongate tube 702, and a pair of connectors 706 at respective ends of the elongate tube 702.

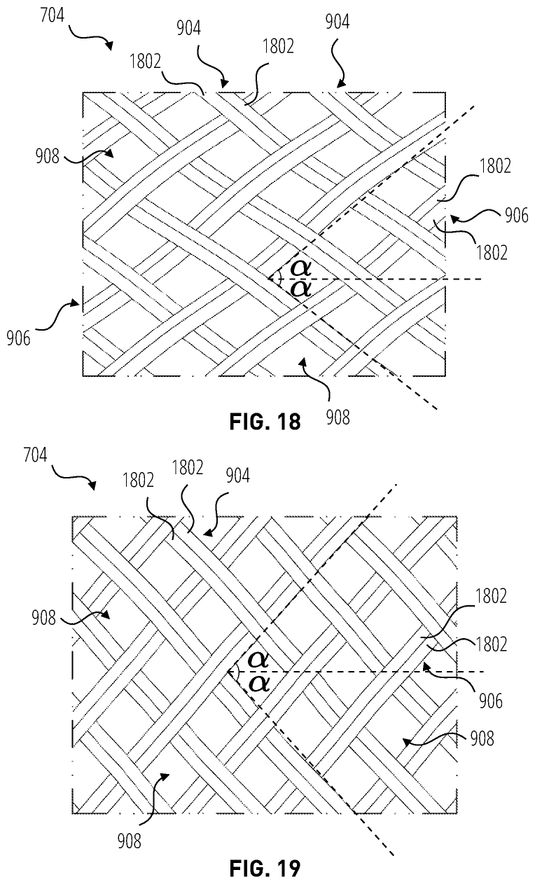

In each of FIG. 7 to FIG. 16, the construction of the conduit 700, and in particular the sheath 704, is in schematic form for clarity. An example construction of the sheath 704 is described in further detail below with respect to FIG. 18 and FIG. 19. Moreover, it is to be appreciated that the sheath 704 may be omitted in other examples.

In other examples, the conduit may have two (or more) elongate tubes. The two elongate tubes may have different properties. The different properties may include one or more of different materials, corrugations, construction, breathability, diameter, insulation, or heating, for example. The two elongate tubes may be arranged end-to-end. They may be secured together, and fluidly coupled, by an intermediate connector. For example, the intermediate connector may be provided at about a mid-point of the conduit, or about a third of the way along the length of the conduit.

Both the elongate tube 702 and the sheath 704, and thus the conduit 700, may be flexible. This may reduce tube drag forces. As described in further detail below, the flexibility of the elongate tube 702 may vary in use. But the sheath 704 may mitigate this.

The conduit 700 may be relatively lightweight. This may reduce tube drag forces. Omission of a water trap may contribute to making the conduit 700 relatively lightweight. In use, accumulation of condensate within a water trap may make a conduit relatively heavy. In some examples, the conduit 700 may have a mass, in an equilibrated state, of between about 130 gm and 170 gm, between about 140 gm and 160 gm, or between about 145 gm and 155 gm, e.g., about 150 grams (gm). In some examples, the conduit 700 may have a mass, in a conditioned state, of between about 260 gm and 300 gm, between about 270 gm and 290 gm, or between about 275 gm and 285 gm, e.g., about 280 gm.

The elongate tube 702 is defined by a tube wall which defines an internal lumen for passage of respiratory gases from one end of the conduit 700 to the other. In the illustrated example, the conduit 700 has a single elongate tube 702 and a single lumen, as opposed to two or more parallel tubes defining two or more parallel lumens. Multiple smaller-diameter tubes may be more prone to one or more of kinking and occlusion, and may be less flexible than an equivalent single tube/lumen.

The tube wall may have a corrugated profile including a plurality of parallel annular corrugations, as described in further detail below. The corrugations may have one or more of the advantages of improving flexibility of the elongate tube 702, promoting controlled deformation and mitigating kinking.

The corrugations may extend along a length of the elongate tube 702 between the connectors 706. Upwards of 80%, or 90%, of the length of the elongate tube 702 may be corrugated. End portions of the elongate tube 702 engaged by the connectors 706, as described in further detail below, may be uncorrugated. The absence of corrugations in the end portions may improve sealing between the elongate tube 702 and the connector 706. In other examples, the tube wall may have a helical profile (e.g., a single helix or double helix). In some examples, one or more portions of the elongate tube 702 between the connectors may be devoid of the corrugated/helical profile.

The tube wall of the elongate tube 702 may be formed from a breathable material (described in further detail below) which is permeable to water molecules. The breathable material may be highly permeable to water molecules. The breathable material permits passage of water molecules through the tube wall to ambient air. The breathable material of the tube wall may reduce the absolute humidity of the respiratory gases within the lumen as they pass along the length of the conduit 700, in use. The breathable material of the tube wall may dissipate condensate or other liquids, e.g., from the patient, which may be present within the lumen. The breathable material may substantially impede the passage of respiratory gases (e.g., nitrogen, oxygen, carbon dioxide and helium) through the tube wall. The breathable material may impede the passage of liquid water droplets through the tube wall. The breathable material may impede the passage of pathogens through the tube wall.

The breathable material in this example forms the entirety of both the length and circumference of elongate tube 702, at least between the connectors 706. In other examples, the breathable material may define only a portion or portions of one or more of the length and circumference of the elongate tube 702. For example, the breathable material may be co-extruded with another, dissimilar (e.g., non-breathable), material. The dissimilar material may provide structural support to the elongate tube 702.

Forming the entirety of the elongate tube 702 from the breathable material advantageously provides a relatively larger surface area of the breathable material, which increases the overall permeability of the elongate tube 702 and effectiveness in mitigating condensate. It may also simplify manufacture of the elongate tube 702. A continuous length of tubing may be extruded and cut to length to form the elongate tube 702, as described in further detail below.

All else being equal, the greater the permeability of the breathable material to water molecules, the more effective the elongate tube 702 may be at dissipating one or more of water vapor and condensate (if any) from within the lumen of the elongate tube 702. But it has been found that, the breathable materials absorb an increasingly larger mass of water molecules, relative to the dry mass of the breathable material, e.g., upwards of about 100% of its own dry mass in water molecules in immersion testing. The mechanical properties of the elongate tube 702 may change significantly during use. For example, an elongate tube configured to absorb about 100% of its own dry mass in water molecules may be made of as much as 50% water during use, and may become significantly more flexible than prior to use.

Because the state of the conduit 700 may vary in use, throughout the detailed description and claims the terms “dry,” “equilibrated,” “conditioned” and “saturated” refer to various different states, or ranges of states, of one or more of the elongate tube 702, sheath 704 and conduit 700. These states are each defined in the Glossary, below.

In one example, it was found that an elongate tube 702 on its own (i.e., without the sheath 704 or other similar structure), made from a breathable material such that the elongate tube 702 was configured to absorb upwards of about 120%, e.g., about 133%, of its own mass in water molecules in immersion testing, was sufficiently stiff to meet the compliance requirements of the ISO 5367:2014(E) standard when in an equilibrated state or when conditioned as set out in the standard. But when that same elongate tube 702 was used to convey humidified respiratory gases for a prolonged period, it was found that the breathable material softened to such an extent that the elongate tube 702, in the conditioned state as defined herein, may no longer meet the same compliance requirement.

Absorption of such a relatively large mass of water molecules by the elongate tube 702, e.g., upwards of 100% of its own dry mass in immersion testing, has also been found to cause the elongate tube 702 to expand appreciably. That is, there is volumetric increase in the breathable material of the elongate tube 702 as it absorbs water molecules. In at least some examples, this expansion may be readily apparent to the naked eye, in use. The dimensions of one or more of the elongate tube and the sheath may vary temporally (i.e., over time, it absorbs and desorbs water molecules), in use.

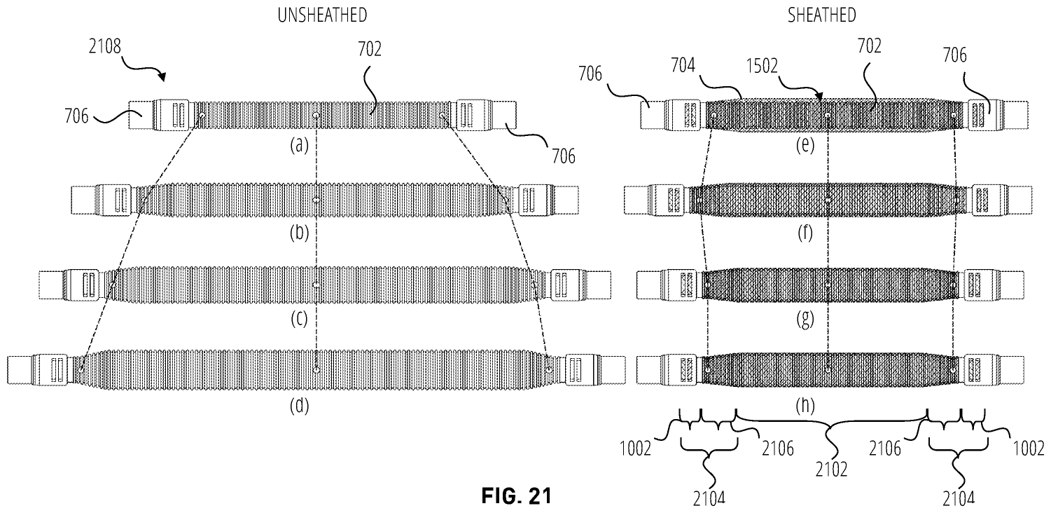

In some examples, one or more of the outside diameter, inside diameter, wall thickness and corrugation pitch (number of corrugations per unit length) of the elongate tube 702, when in a conditioned state, may vary along the length of the elongate tube 702. Whereas in one or more of a dry state, equilibrated state and saturated state, one or more, and potentially all, of the outside diameter, inside diameter, wall thickness and corrugation pitch of the elongate tube 702 may be substantially uniform along at least a central portion of the elongate tube 702. The central portion may exclude end portions of the elongate tube 702. The end portions may consist of the respective cuff portion and respective tapered portion (if any) of the elongate tube 702 which may taper towards the connectors 706 (refer to the central portion 2102, end portions 2104, cuff portions 1002 and tapered portions 2106 illustrated in FIG. 21(h)). Expansion of the end portions may be constrained by the respective connectors 706.

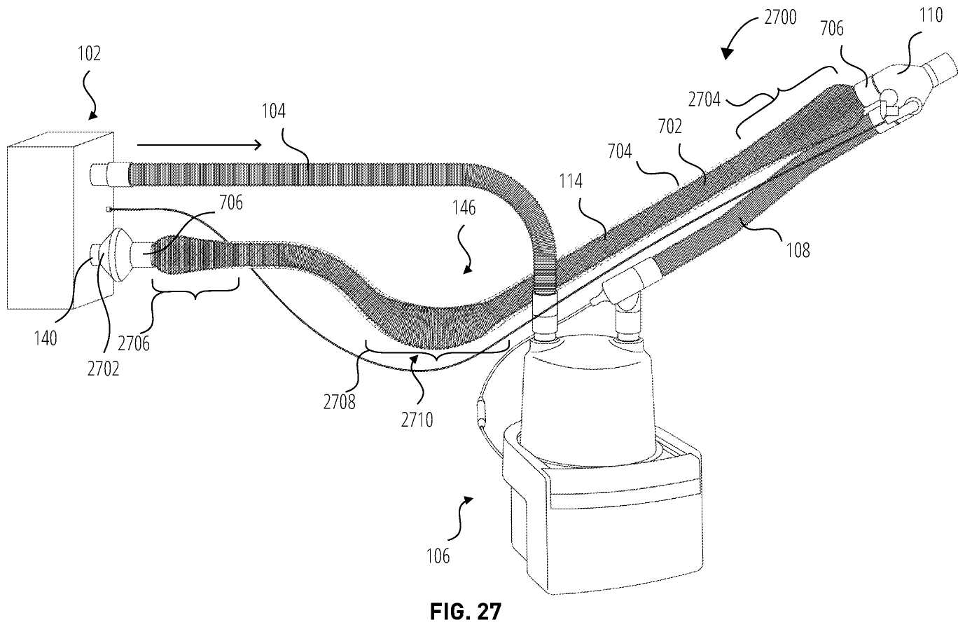

Although expansion of the elongate tube 702 may be non-uniform in use (e.g., in the conditioned state), the following description of FIG. 7 to FIG. 26 assume that changes in dimensions of the elongate tube 702 and the sheath 704 occur generally uniformly for ease or representation and explanation. Non-uniform absorption and expansion of the conduit 700 is described in further detail with respect to FIG. 27.

The diameter, length, wall thickness and other dimensions of test specimens may be measured using a non-contact method using computerized tomography (CT) scanning or an optical measurement system. In some examples, a contact-based measuring method may be used, e.g., with coordinate measuring machine (CMM).

In immersion testing (described in the Glossary, below), sample specimens of the elongate tube 702, in isolation from the sheath (i.e., without the sheath 704 or other such constraint), may expand by between about 20% and 70% in one or more of a radial direction (e.g., a maximum outside diameter, minimum inside diameter, or average outside or inside diameter, for a corrugated elongate tube 702), a longitudinal direction (i.e., length), and a wall thickness of the tube wall, from a dry state to a saturated state. In some examples, sample specimens of the elongate tube 702 may expand by between about 20% and 70%, between about 25% and 50%, or between about 30% and 50%, in one or more of the radial direction, the longitudinal direction, and the wall thickness. In one example, sample specimens of the elongate tube 702 of the example conduit 700 were found to expand in the radial direction by about 42%, longitudinal direction by about 37% and wall thickness by about 34%. In another example, sample specimens of the elongate tube 702 were found to expand in each of the radial direction, longitudinal direction and wall thickness by about 32% each.

In some examples, a wall thickness of the elongate tube, in a dry state, may be between about 0.5 mm and 0.9 mm, between about 0.6 mm and 0.8 mm, between about 0.65 mm and 0.75 mm, between about 0.68 mm and 0.72 mm, or between about 0.69 mm and 0.71 mm, e.g., about 0.70 mm. In a saturated state in isolation from the sheath, the same tube wall may have a wall thickness of between about 0.7 mm and 1.1 mm, between about 0.8 mm and 1.0 mm, between about 0.85 mm and 0.95 mm, between about 0.90 mm and 0.94 mm, or between about 0.91 mm and 0.93 mm, e.g., about 0.92 mm.

In some examples, a maximum outer diameter of the elongate tube, in a dry state, may be between about 20 mm and 26 mm, between about 21 mm and 25 mm, or between about 22 mm and 24 mm, e.g., about 23 mm. In a saturated state in isolation from the sheath, the same elongate tube may have a maximum outer diameter of between about 25 mm and 35 mm, between about 28 mm and 32 mm, or between about 29 mm and 31 mm, e.g., about 30 mm.

Referring still to FIG. 7, the sheath 704 is provided about the elongate tube 702. The sheath 704 is shown in cutaway for illustrative purposes, but in this example extends continuously between the connectors 706 at opposing ends of the conduit 700. The sheath 704 is illustrated as being generally tubular in shape (that is, circular in cross-section), but may not necessarily have a perfectly circular cross-section. In particular, it will be appreciated that the sheath 704 may be formed by being cut to length from a continuous length of flexible sheathing material which may be stored in the form of a roll of flattened sheathing. For example, the sheath 704 may tend to adopt more of an elliptical or pointed oval cross-section when provided about the elongate tube 702. Moreover, the sheath 704 may not necessarily have a uniform shape in cross-section along its length as is illustrated in the drawings.

In the illustrated example, ends the sheath 704 is secured relative to the ends of the elongate tube 702 by the connectors 706, as described in further detail below. The elongate tube 702 and the sheath 704, in this example, are otherwise not secured to each other intermediate the connectors 706. In other examples, the elongate tube 702 and the sheath 704 may be further secured with respect to each other at one or more discrete points along the length of the conduit intermediate the connectors 706, such as at about the mid-point or about a third of the way along the length of the elongate tube 702, for example. In yet other examples, the sheath 704 may extend only partway along the length of the elongate tube 702. In one example, opposing ends of the sheath 704 may be secured to the elongate tube 702 by one of the connectors 706 and an additional intermediate connector.

In yet other examples, the conduit may have two or more sheaths along the length of the conduit. The two or more sheaths may be discrete from each other. The two or more sheaths may have different properties. The different properties may include one or more of a different material, braiding pattern, number of braiding elements, and length/diameter gradient. The two or more sheaths may be arranged substantially end-to-end with each other. The two or more sheaths may be secured to the elongate tube 702, by an intermediate connector. The intermediate connector may be provided at about a mid-point of the conduit or elsewhere (such as closer to the patient end). The intermediate connector may also secure together, and fluidly couple, two discretely-formed elongate tubes. One, or both, of the discretely-formed elongate tubes may be formed, at least in part, from the breathable material. The sheath 704 may extend along an entire length of the conduit 700 between the end connectors 706. Or the sheath 704 may be provided about only the breathable elongate tube 702.

The sheath 704 has a plurality of braiding elements braided into an open mesh-like tubular configuration defining a plurality of openings, as described in further detail below with reference to FIG. 18 and FIG. 19. The sheath 704 may be highly porous and vapor-permeable, and therefore does not inhibit the passage of water molecules through the breathable material from within the lumen of the elongate tube 702 to ambient air. That is, water vapor and other gases may pass freely through the sheath 704. The sheath 704 may have negligible effect, or no effect, on the temperature and humidity of the ambient gases adjacent the outside of the tube wall, so as not to effect the vapor pressure gradient across the tube wall. The open mesh-like structure of the sheath 704 may also permit visual inspection of the elongate tube 702.

When the example conduit 700 is in one or more of a dry state and an equilibrated state, the sheath 704 may lie loosely about the elongate tube 702. That is, an inside diameter of the sheath 704 may exceed a corresponding outside diameter of the elongate tube 702, such that the sheath 704 is at least partially spaced from the elongate tube 702. The elongate tube 702 may be able to move relative to the sheath 704 (or vice versa).

“At least partially spaced” is intended to allow for some contact between the sheath 704 and the elongate tube 702 about or along one or more of the circumference of the conduit 700 and the length of the conduit 700. That is, the sheath 704 need not (but may) be entirely spaced from the elongate tube 702. In the case of a corrugated tube, “at least partially spaced” refers to a spacing from the peak of a corrugation, rather than merely a spacing from a trough between two peaks.

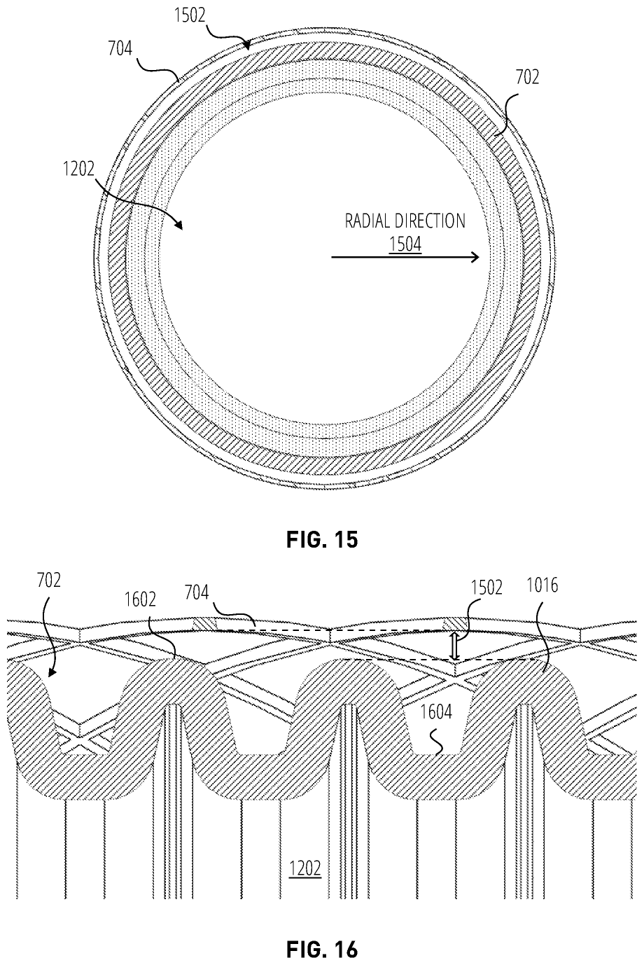

The sheath 704 may be described or illustrated as concentric or coaxial with respect to the elongate tube 702, as shown in FIG. 15 for example, but it will be appreciated that, in practice, relative movement may occur between the elongate tube 702 and the sheath 704 in one or more of the dry state and the conditioned state. The elongate tube 702 and the sheath 704 will not necessarily share the same center in cross-section. For example, the elongate tube 702 may drape towards the bottom of the sheath 704 or, conversely, the sheath 704 may drape towards the top of the elongate tube 702. It will be appreciated that the spacing between the elongate tube 702 and the sheath 704 will therefore not necessarily be uniform in either the circumferential direction or the longitudinal direction of the conduit 700. And the sheath 704 may contact the elongate tube 702 in places, while still being considered generally partially spaced.

The elongate tube 702 and the sheath 704 are shown as having relatively uniform diameters along their length. But in use, and particularly in the conditioned state (as discussed above), one or more of the elongate tube 702 and the sheath 704 may vary in diameter or shape along their length as described in further detail below with respect to FIG. 27. In one example, in a conditioned state the sheath 704 may engage a full circumference of the elongate tube 702 in a first portion of the conduit 700, but be spaced from an entirety, or at least a portion, of the circumference of the elongate tube 702 at a second portion of the conduit 700. In that example, the sheath 704 may be said to be at least partially spaced from the elongate tube 702 in the second portion. Or the sheath 704 may be said to be at least partially spaced from the elongate tube 702 as a whole. But the sheath 704 is not regarded as at least partially spaced from the elongate tube 702 in the first portion of the conduit 700 in particular.

The sheath 704 may interact with the elongate tube 702 as the elongate tube 702 expands in one or more of the radial direction, the longitudinal direction and wall thickness due to absorption of water molecules in use, as described in further detail below. In particular, the sheath 704 may constrain expansion of at least a portion of the elongate tube 702 in at least one of the longitudinal and radial directions of the conduit 700, when the conduit 700 is in a constrained conditioned state. That is, the sheath 704 restricts expansion of the elongate tube 702 by applying a counteracting force under at least some conditions.

The sheath 704 may also provide a degree of physical protection to the elongate tube 702, e.g., one or more of strain relief, abrasion resistance and distribution of point or localized loads, e.g., across multiple corrugations of the elongate tube 702.

The connectors 706 of the conduit 700 may provide for convenient and reliable pneumatic coupling of the elongate tube 702 to other components of a respiratory assistance system to convey respiratory gases between those other components. In particular, where the conduit 700 is intended for use as the expiratory conduit 114 in the respiratory assistance system 100, the connectors 706 are configured for pneumatic connection directly with an outlet of the Y-piece 110 and the gases return inlet 140 of the gases source 102, respectively. In some examples, a filter may be provided between the conduit 700 and the gases return inlet 140.

In the illustrated example the connectors have a standard medical taper for interoperability with a wide variety of respiratory assistance systems and components thereof.

In some examples, in particular where the conduit has a heater wire, at least one of the connectors may include a socket for establishing an electrical connection with the humidifier 106, the gases source 102, or another power source.

The connectors 706 each define a bore which is fluidly coupled with the lumen of the elongate tube 702.

FIG. 8 provides a detailed isometric view of one end of a conduit 700 according to the present disclosure, including a connector 706, an end portion of the sheath 704 and an end portion of the elongate tube 702 (partially obscured by the sheath 704 and the connector 706). Also shown is the bore 802 of the connector 706, which is fluidly coupled with the lumen of the elongate tube 702.

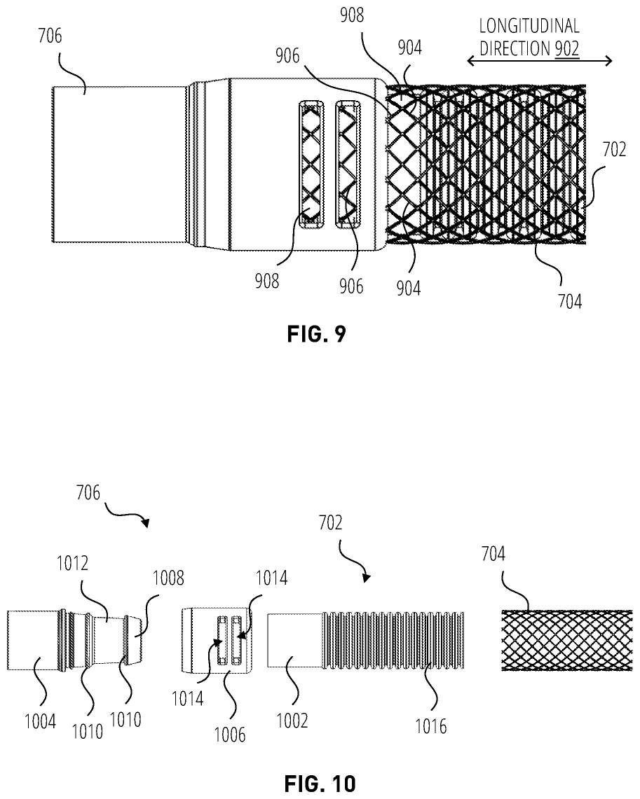

FIG. 9 provides a detailed side view of an end of a conduit 700 according to the present disclosure. Also illustrated is what is referred to as the longitudinal direction of the conduit 700, represented by the arrow 902. It will be appreciated that the conduit 700, in use, may not necessarily adopt a strictly linear configuration as shown in the drawings, and references to “longitudinal,” “axial” and the like are not to be construed as being limited to such a linear configuration.

The sheath 704 may be a braided tubular sheath as described in further detail below with respect to FIG. 18 and FIG. 19.

FIG. 10 provides an exploded detail view of an end of a conduit 700 according to the present disclosure, including one of the connectors 706 and portions of the elongate tube 702 and sheath 704. As shown, the connector 706 in this example has a first connector component 1004 and a second connector component 1006.

The first connector component 1004 may have a chamfered edge 1008. The chamfered edge 1008 may aid insertion of the first connector component 1004 into the lumen 1202 of the elongate tube 702.

The first connector component 1004 may have at least one flange 1010. In some examples, the first connector component 1004 may have three flanges 1010. The flanges 1010 may extend circumferentially about the first connector component 1004. The flanges 1010 may resist inadvertent disassembly of the first connector component 1004 from one or more of the elongate tube 702, the sheath 704 and the second connector component 1006 (or vice versa). The flange 1010 may extend radially outwardly from at least a portion of the first connector component 1004, and may in part define an annular channel 1012. The second connector component 1006 may in part be received within the annular channel 1012. The flange 1010 may inhibit one or more of the elongate tube 702, the sheath 704 and the second connector component 1006 from sliding off the first connector component in the longitudinal direction.

The second connector component 1006 may have one or more apertures 1014. The apertures 1014 may be arranged in pairs on opposing sides of the second connector component 1006. The apertures 1014 may be through-holes. That is, the apertures may each extend all the way through a cylindrical wall defining part of the second connector component 1006.

The apertures 1014 may aid manufacturing of the conduit 700, as described in further detail below. The apertures 1014 may provide increased grip for a user to connect or disconnect the conduit 700 to/from other components of the respiratory assistance system.

Although the apertures 1014 of example conduit 700 are arranged as pairs of parallel elongate slots, there may alternatively be any number of apertures in any suitable shape or arrangement. For example, the apertures may alternatively form a chevron pattern or a logo. In some examples, the chevron pattern or other indicia (such as text or symbols) may indicate a preferred flow direction, or a specific component of the respiratory assistance system to which the respective connector 706 is intended to be coupled. This may reduce the risk of misconnections. The example conduit 700 may be symmetric and may be configured to be coupled between the gases source 102 (or filter) and the Y-piece 110 in either direction. In other examples, the conduit may be directional.

At least one of the first connector component 1004 and the second connector component 1006 may be formed from a polymer material. The polymer material may include any one or more of polycarbonate (PC), polypropylene (PP) and polyethylene (PE). In some examples, the first connector component 1004 and the second connector component 1006 may be formed from the same material. In other examples, the first connector component 1004 and the second connector component 1006 may be formed from different materials.

The first connector component 1004 may be injection molded. The second connector component 1006 may be overmolded to one or more of the elongate tube 702, the sheath 704 and the first connector component 1004. In some examples, the second connector component 1006 may be overmolded to all three of the elongate tube 702, the sheath 704 and the first connector component 1004.

In other examples, the sheath 704 may alternatively, or additionally, be secured to one or more of the connector 706 and the elongate tube 702 by ultrasonic welding, radio frequency (RF) welding, an adhesive, a hose clamp, a clip (e.g., a circlip or snap-fit clip) or the like.

Also shown in FIG. 10 is a cuff portion 1002 and a corrugated portion 1016 of the elongate tube 702. The cuff portion 1002 in this example, which is intended to be received between the first connector component 1004 and the second connector component 1006 of the connector 706, is not corrugated. The absence of corrugations from the cuff portion 1002 may have one or more of the advantages of aiding insertion of the chamfered edge 1008 within the cuff portion 1002, facilitating a more secure engagement between the elongate tube 702 and the connector 706, and reducing the potential for pneumatic leaks between the elongate tube 702 and the connector 706.

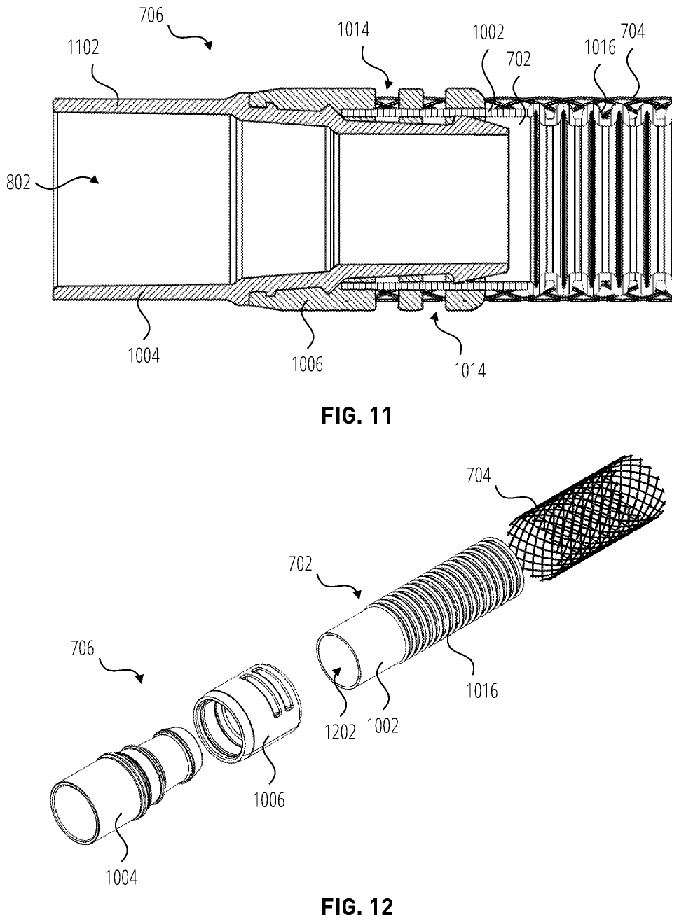

FIG. 11 is a cross-sectional view an end of a conduit 700 according to the present disclosure. The sheath 704 is represented schematically for clarity. It should be appreciated that the sheath 704 may taper down to the diameter, e.g., the internal diameter, of the connector 706 as shown in FIG. 21(e), for example. Due to the nature of a braided tubular mesh sheath, this can be accomplished by extending the length of the sheath in the portion requiring a smaller diameter.

The first connector component 1004 defines the bore 802 of the connector 706. The first connector component 1004 may include a male or female tapered conical end 1102 configured to connect to other components of the respiratory assistance system, e.g., the gases return inlet 140. An opposing end of the first connector component 1004 may be partially received within the lumen 1202 of the elongate tube 702, e.g., at the cuff portion 1002. One or more of the elongate tube 702 and the sheath 704 may be exposed, e.g., visible, through the apertures 1014 of the second connector component 1006 of the connector 706.

As shown, the end of one or more of the elongate tube 702, e.g., the cuff portion 1002, and the corresponding end of the sheath 704 may be secured relative to each other by one or more of the first connector component 1004 and the second connector component 1006. The ends of the elongate tube 702 and the sheath 704 may coincide within the connector 706. In some examples, one or more of the elongate tube 702 and the sheath 704 may be secured between (e.g., “sandwiched” by) the first connector component 1004 and the second connector component 1006. In some examples, the sheath 704 may be positioned directly adjacent the elongate tube 702 within the connector 706. In other examples, as shown, the sheath 704 may be spaced from the elongate tube 702 within the connector 706.

FIG. 12 provides an isometric exploded detail view of an end of a conduit 700 according to the present disclosure, including the elongate tube 702, sheath 704, and the first connector component 1004 and second connector component 1006 of the connector 706. Also shown are the cuff portion 1002 and the corrugated portion 1016 of the elongate tube 702, and a portion of the lumen 1202 for passage of respiratory gases from one end of the conduit 700 to the other.

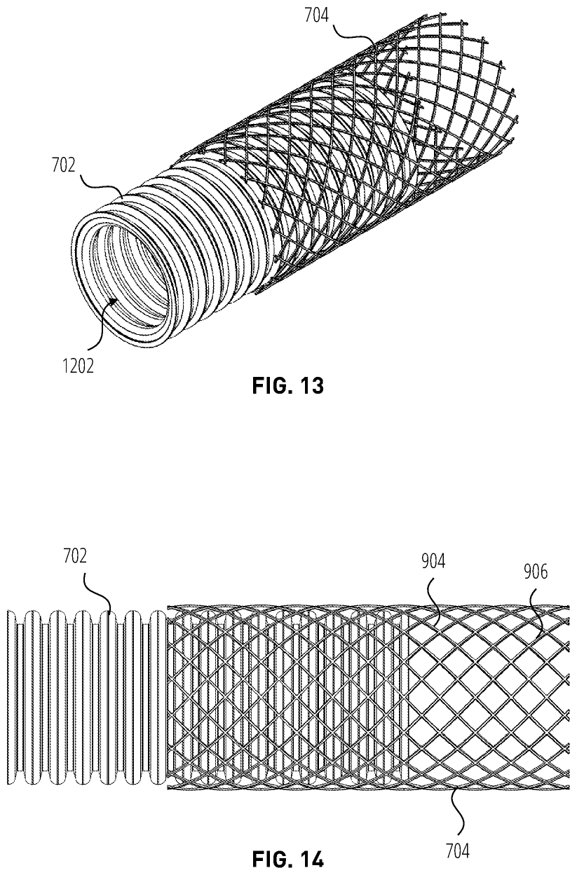

FIG. 13 is a detailed, partially exploded, isometric view of portions of an elongate tube 702 and the sheath 704 of a conduit 700 according to the present disclosure. Also shown (in part) is the lumen 1202 defined by the elongate tube 702.

FIG. 14 is a side view, partially exploded, of portions of the elongate tube 702 and the sheath 704 of a conduit 700 according to the present disclosure.

The sheath 704 is in schematic form for clarity. It shows the plurality of clockwise braiding elements 904 and counter-clockwise braiding elements 906 of the sheath 704. The clockwise braiding elements 904 and the counter-clockwise braiding elements 906 may be interleaved as described in further detail below and illustrated in FIG. 18 and FIG. 19.

FIG. 15 shows a cross-section through the elongate tube 702 and the sheath 704 of a conduit 700 according to the present disclosure. The connector 706 is omitted for clarity.

It can be observed that may be a spacing 1502 between the outer surface of the elongate tube 702 and the inner surface of the sheath 704 when the example conduit 700 is in an equilibrated state, as illustrated. This allows for some expansion of at least a portion of the elongate tube 702 in at least one of the radial direction 1504 and the longitudinal direction 902. But, under some conditions, further expansion of the elongate tube 702 in the radial direction 1504 may become constrained by the sheath 704, as described in further detail below. That is, the sheath 704 may selectively constrain expansion of the elongate tube 702 in this example.

As explained above, the spacing 1502 need not necessarily be uniform or even continuous around the entire circumference of the conduit 700, or along the length of the conduit 700 (in the longitudinal direction 902). Nor will the sheath 704 in particular necessarily have a perfectly circular cross-section or be concentric with the elongate tube 702.

FIG. 15 also illustrates the lumen 1202 defined by the tube wall, and an example of what is referred to as the radial direction of the conduit 700, represented by the arrows 1504.

FIG. 16 is a detailed cross-sectional side view of portions of a conduit 700 according to the present disclosure. A sheath 704 and the corrugated portion 1016 of an elongate tube 702 are shown. The drawing depicts only an upper portion of a length of the conduit 700 in a horizontal orientation, with the lumen 1202 towards the lower part of the drawing and ambient air at the upper part of the drawing.

A spacing 1502 can be observed between the outer surface of the elongate tube 702, at the peaks 1602 of the corrugations, and the inner surface of the sheath 704. It can also be observed that troughs 1604 of the corrugated portion 1016 in this example are flattened. That is, the troughs are substantially cylindrical. By contrast, the peaks 1602 may curve continuously, as shown.

### Breathable Material

The breathable material may be, or include, a block copolymer such as a thermoplastic elastomer (TPE) with both ‘hard segments’ and ‘soft segments.’ In some examples, the hard segments may be a hard, rigid, semi-crystalline polymer which may be hydrophobic (for example, a polyester such as polybutylene terephthalate (PBT) or similar). In some examples, the soft segments may be a soft, flexible, amorphous and hydrophilic polymer (for example, an ether type macro glycol). The breathable material may combine the hydrophilic properties of the soft segments with the mechanical properties of the hard segments.

In some examples, the block copolymer may be at least about 90%, at least about 92%, at least about 95%, or at least about 97%, e.g., about 97% or about 98.5%, of the breathable material of the elongate tube by one or more of mass, weight and volume.

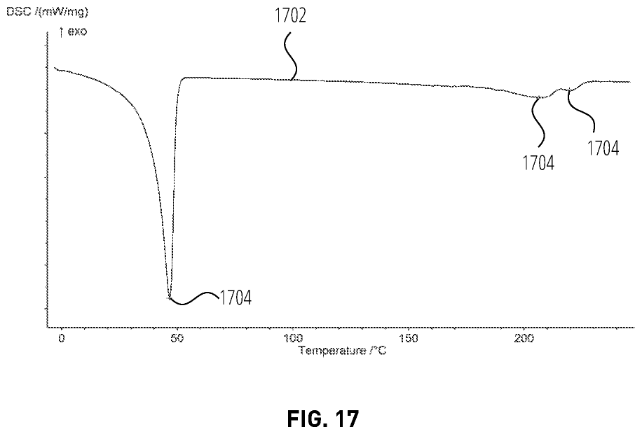

FIG. 17 is a graph showing the results of differential scanning calorimetry (DSC) testing (defined in the Glossary below) on sample specimens from an example elongate tube 702 formed from a breathable material in accordance with the present disclosure.

The line 1702 in FIG. 17 shows the differential heat rate (measured in milli-Watts per milligram, mW/mg) of the example breathable material as it is heated a second time during DSC testing.

The peaks in the line 1702 represent the melting points 1704 of the breathable material. The term “melting point” as used herein refers to the temperature corresponding to a peak in the DSC graph. It will be appreciated that melting may begin before the peak.

In some examples, the breathable material may have two, three, or more melting points 1704. The lowest melting point 1704 of the breathable material may be at a temperature greater than about 37° C., greater than about 42° C., greater than about 44° C., greater than about 45° C., or greater than about 46° C., e.g., at a temperature of about 47° C. The lowest melting point 1704 temperature of the breathable material may be less than about 108° C., less than about 100° C., less than about 80° C., less than about 60° C., or less than about 50° C. In some examples, the lowest melting point 1704 may be at a temperature between about 37° C. and 100° C., between about 40° C. and 60° C., or between about between about 40° C. and 50° C.

In some examples, the breathable material may have a melting point 1704 at a temperature between about 42° C. and 52° C., between about 44° C. and 50° C., between about 45° C. and 49° C., or between about 46° C. and 47° C., e.g., at a temperature of about 47° C.

In some examples, the breathable material may have a melting point 1704 at a temperature between about 202° C. and 212° C., between about 204° C. and 210° C., between about 205° C. and 209° C., or between about 206° C. and 208° C., e.g., at a temperature of about 207° C.

In some examples, the breathable material may have a melting point 1704 at a temperature between about 215° C. and 225° C., between about 217° C. and 223° C., between about 218° C. and 222° C., or between about 219° C. and 221° C., e.g., at a temperature of about 220° C.

In some examples, the breathable material may have two melting points 1704 above a temperature of about 197° C., above about 202° C., above about 204° C., above about 205° C., or above about 206° C., e.g., at and above a temperature of about 207° C.

In some examples, the breathable material may have two melting points 1704 at temperatures between about 202° C. and 225° C., between about 204° C. and 223° C., between about 205° C. and 222° C., or between about 206° C. and 221° C., e.g., at temperatures of about 207° C. and 220° C.

In some examples, the breathable material may have two melting points 1704 with a temperature difference of less than about 23° C., less than about 18° C., less than about 16° C., less than about 15° C., or less than about 14° C., e.g., a temperature difference of about 13° C.

In some examples, the breathable material may have two melting points 1704 with a temperature difference of between about 150° C. and 170° C., between about 155° C. and 165° C., between about 158° C. and 162° C., or between about 159° C. and 161° C., e.g., a temperature difference of about 160° C.

The breathable material may have three melting points 1704. The three melting points may be at temperatures of between about 42° C. and 225° C., between about 44° C. and 223° C., between about 45° C. and 222° C., or between about 46° C. and 221° C.

In one particular example, the three melting points 1704 may be at temperatures of about 47° C., 207° C. and 220° C. (all ±5° C., ±3° C., ±2° C. or ±1° C.).

In some examples, the breathable material may have a lowest melting point at a temperature greater than 37° C. (or 42° C., 44° C., 45° C., 46° C.). As described above, 37° C. may correspond to one or more of the temperature of the respiratory gases delivered to the patient by the respiratory assistance system and the temperature of respiratory gases expired by the patient.

The lowest melting point of the breathable material may correspond to the melting point of the soft segments of the breathable material. It has been found that selection of a breathable material with all melting points above this temperature may mitigate melting of the soft segments of the breathable material when the conduit conveys heated and humidified respiratory gases in use. At temperatures above the melting point of the soft segments, the soft segments may remain bound to the hard segments. It is not apparent to a user that there is any “melting” of the elongate tube. But it has been found that below the lowest melting point, a majority of the soft segments may remain solid during use and contribute to the structural integrity of the elongate tube. This may enable the use of a higher ratio of soft segments to hard segments in the breathable material, which in turn may increase the breathable material's permeability to water molecules.

In some examples, the temperature of the respiratory gases within the lumen of the inspiratory conduit 108 may be higher than 37° C. to allow for a temperature drop before the respiratory gases are delivered to the patient via the patient interface. In some examples, the temperature of the respiratory gases within the lumen of the inspiratory conduit 108 may be as high 50° C. or 60° C., for example. Accordingly, in some examples the lowest melting point of the breathable material may be at a temperature greater than 50° C., or greater than 60° C.

It has been found that such a breathable material is particularly suited for use in conduits omitting a heater, e.g., a heater wire. A heater wire may be heated to a temperature well above the temperature of the respiratory gases within the lumen 1202. It has been found that this heating may melt at least some of the soft segments of the breathable material during use, even if the temperature of the respiratory gases does not exceed the lowest melting point. This is particularly the case if the heater wire is embedded in the tube wall or pressed against the tube wall. By omitting the heater wire and selecting a breathable material with melting points above an anticipated, or actual, maximum temperature of the respiratory gases, at least a majority of the soft segments will remain solid during use of the conduit, and thus contribute to the mechanical strength of the conduit, in use. In some examples, a sheath may not be required.

In other examples, the conduit may include a heater wire and the breathable material may be selected to have a lowest melting point above the anticipated, or actual, maximum temperature of the heater wire, in use. In other examples, the conduit may be operated, e.g., by controlling a duty cycle of the heater wire, so that the temperature of the heater wire does not exceed the lowest melting point of the breathable material. In other examples, the elongate tube 702 may be formed from a breathable material and a non-breathable material, and the heater wire may be embedded in, or pressed against, the non-breathable material.

At least a portion of the elongate tube 702 may be configured to absorb more than about 45%, more than about 65%, more than about 75%, more than about 100%, more than about 120%, or more than about 130% of its own mass in water, in immersion testing (described in the Glossary, below).

In some examples, samples of at least a portion of the elongate tube 702 may be configured to absorb between about 45% and 250%, between about 65% and 200%, between about 75% and 175%, between about 100% and 160%, between about 110% and 150%, between about 120% and 140%, between about 130% and 140%, or between about 133% and 139%, e.g., about 139%, of their own dry mass in water molecules, in immersion testing.

In some examples, samples of at least a portion of the elongate tube 702 may be configured to absorb between about 40% and 80%, between about 40% and 60%, between about 45% and 55%, or between about 48% and 51%, e.g., about 49%, of their own dry mass in water molecules, in immersion testing.

In some examples, samples of at least a portion of the elongate tube 702 may be configured to absorb between about 100% and 250%, between about 100% and 180%, between about 110% and 150%, between about 120% and 140%, between about 130% and 145%, or between about 135% and 145%, e.g., about 138% or 139%, of their own dry mass in water molecules, in immersion testing.

In some examples, samples of at least a portion of the elongate tube may be configured to absorb more than about 33%, between about 33% and 200%, between about 100% and 160%, between about 120% and 140%, or between about 130% and 135%, e.g., about 133% of its own mass in water molecules, in immersion testing.

The permeability to water molecules of the breathable material described herein has been found to mitigate condensate within the lumen to such an extent that at least one of a heater wire and a water trap may be omitted. In some examples, both the heater wire and the water trap may be omitted.

In some examples, it has been found that such an elongate tube 702 may be sufficiently permeable to water molecules to passively (i.e., without heating, emptying a water trap, or draining the conduit) mitigate condensation within the lumen after prolonged use of the conduit 700, e.g., continuous use for about 24 hours. In some examples, there may be no net accumulation of condensate within the lumen, including condensate from other sources such as the catheter mount, after prolonged use. In some examples, conduits according to the present disclosure may be sufficiently breathable that there is no need for a medical professional to drain condensate from the conduit after prolonged use, at least when the conduit is used in a regulated environment, e.g., ambient air within the range of about 20° C. to 24° C. and about 40% to 60% relative humidity.

In one example, it was found that a conduit having an elongate tube 702 configured to absorb between about 45% and 55%, between about 46% and 52%, between about 47% and 51%, or between about 48% and 50%, e.g., about 49% or about 50%, of its own mass in water molecules in immersion testing, was suitable for use as an expiratory conduit 114 without the need for a heater wire or a water trap in at least some ambient conditions. Nor did it require a sheath 704 or other such measures to compensate for softening of the elongate tube 702 as it absorbs water molecules, in use. That is, the elongate tube alone was found to meet one or more of the resistance to flow and compliance requirements of ISO 5367:2014(E) in a conditioned state without a sheath 704.

In another example, it was found that a conduit 700 having an elongate tube 702 configured to absorb between about 130% and 140% of its own mass in water molecules, in immersion testing, also without a heater wire or a water trap, was suitable for use as an expiratory conduit 114. This example was found to better mitigate condensation across a wider range of ambient conditions when compared to the previous example (configured to absorb between about 45% and 55%). The conduit 700 was provided with a sheath 704. The conduit 700 was found to meet one or more of the resistance to flow and compliance requirements of ISO 5367:2014(E) in a conditioned state despite absorbing a far greater proportion of water molecules.

In one example, after prolonged use, e.g., about 24 hours, no condensate was found to have accumulated at the gases source or within the lumen of the conduit 700 when used as the expiratory conduit 114. After five days of continuous use, condensate was found to accumulate at the gases source at a rate of less than about 1 milliliter per hour (ml/hr) and within the lumen of the conduit 700 at a rate of less than about 2 ml/hr, without a heater wire or water trap. But in some examples, the conduit may optionally be provided with at least one of a heater wire and a water trap.

In some examples, a compliance of the conduit 700 may be dependent on a physical interaction between the elongate tube 702 and the sheath 704. The compliance of the conduit 700, when conditioned for a prolonged period, may differ by upwards of 10%, 20% or even 30% when sheathed versus unsheathed.

The breathable material may further include one or more additives. The additives may include one or more of a foaming agent, colorant, ultraviolet (UV) stabilizer, UV absorber, or processing aid, for example. In some examples, the additive(s) may form up to about 10%, up to about 8%, up to about 5%, or up to about 3%, e.g., about 3% or 1.5%, of the breathable material of the elongate tube by one or more of mass, weight and volume. It will be appreciated that the additives themselves may not necessarily be breathable, but the breathable material as whole remains breathable with the additives.

In some examples, the breathable material may be unfoamed. In other examples, the breathable material may be foamed. The breathable material may include a foaming agent additive. The foaming agent may be selected and dosed so that the breathable material is a closed-cell foam. It has been found that voids formed within a foamed breathable material may increase the mass of water which may be absorbed by the breathable material. In one example, a sample of foamed breathable material was found to absorb about double the mass of water when compared to an unfoamed sample of equivalent dry mass and structure (e.g., corrugation profile), in immersion testing. The voids may also improve the thermal insulation provided by the tube wall.

### Sheath

FIG. 18 provides a detailed diagram of a sheath 704 of a conduit 700 according to the present disclosure, with the conduit 700 in an equilibrated state. The sheath 704 is illustrated as if the conduit is laid out horizontally.

The sheath 704 may be less resilient than the elongate tube 702 so that if the elongate tube 702 is crushed, recovery of the elongate tube 702 is not constrained by the sheath 704.

The sheath 704 may be a mesh. The sheath 704 may have a plurality of braiding elements interleaved to form a braided tubular mesh.

In some examples, the sheath may have a bi-axial configuration. The braiding elements may each be arranged helically about the elongate tube 702 in one of a clockwise or anti-clockwise direction.

Each of the clockwise braiding element 904 may be interleaved with each of the counter-clockwise braiding element 906, and vice versa. In some examples, the braiding elements may be interleaved in a regular braid pattern in which a clockwise braiding element 904 passes under two counter-clockwise braiding elements 906 then over two counter-clockwise braiding elements 906, as shown. In other examples, the braiding elements may be interleaved in a diamond braid pattern in which a clockwise braiding element 904 passes under a single counter-clockwise braiding element 906 then over a single counter-clockwise braiding element 906.

The braiding elements of braided tubular mesh sheath 704 may each be orientated at a braid angle α with respect to the longitudinal direction of the conduit 700 (the horizontal direction, as illustrated). In some examples, the braid angle α may be the same for the clockwise braiding elements 904 and the counter-clockwise braiding elements 906. In some examples, the braid angle α in the equilibrated state may be between about 35° and 55°, or between about 40° and 50°. In one example, the braid angle may be about 45°. In some examples, the angle at an intersection between a clockwise braiding element 904 and a counter-clockwise braiding element 906 may be between about 70° and 110°, or between about 80° and 100°. In one example, the angle at the intersection may be about 90°.

In other examples, braiding elements extending in the longitudinal direction, parallel with the elongate tube 702, may be advantageous in applications where expansion of the conduit in the longitudinal direction is undesirable. Braiding elements extending in a circumferential direction may be advantageous in applications where expansion of the conduit in the radial direction is undesirable.

In some examples, the sheath may have a tri-axial or other configuration.

In some examples, the respective braiding elements 904, 906 are not bonded or otherwise permanently joined to each other where they intersect (except where the sheath 704 is secured relative to the elongate tube 702, e.g., at the connectors 706). That is, the braiding elements 904, 906 may move relative to each other to some extent (subject to frictional forces).

The sheath 704 may have an open weave. The clockwise braiding elements 904 may be spaced apart from each other about the circumference of the sheath 704. The clockwise braiding elements 904 may be spaced substantially equidistantly, e.g., adjacent the connector 706. The counter-clockwise braiding elements 906 may be similarly spaced from each other.

The spacing of the braiding elements in the braided sheath forms a plurality of openings 908 between the braiding elements. Each of the openings may be substantially quadrilateral in shape, e.g., have a parallelogram shape or rhomboid shape. The geometry of the openings may vary in use, undergoing an affine transformation as the dimensions of the sheath 704 vary in use as described in further detail below.

In this equilibrated state the openings 908 may be relatively elongated in the longitudinal direction (the horizontal direction, as illustrated) as compared to the tangential direction (the vertical direction, as illustrated).

If the braiding elements 904, 906 are not bonded or otherwise permanently joined together where they intersect, there may be some relative movement between them as the elongate tube 702 expands and/or contracts, in use. It will be appreciated that the spacing between adjacent braiding elements may not necessarily be uniform along the length of the conduit. Nor will all of the openings 908 necessarily form a perfect parallelogram.

In some examples, a useful property of the sheath 704 is that the overall length (in the longitudinal direction) and the diameter (in the radial direction) of the sheath 704 may vary in an inverse relationship. That is, the sheath 704 will tend to contract in the radial direction as it is elongated in the longitudinal direction. Conversely, the sheath 704 will tend to contract in the longitudinal direction as it is expanded in the radial direction. It has been found that this property may be exploited to at least partially counteract changes in the mechanical properties of the elongate tube 702 as it expands due to absorbing water molecules in use, as described in further detail below.

In practice, sheathing may have limits in one or more of the minimum and maximum diameters it can be deformed to. In some examples, e.g., for an adult conduit, the sheath 704 may have an inside diameter which may vary between at least about 23 mm and 43 mm (±2 mm). That is, the sheath 704 may be configured to conform to rods with outside diameters ranging between about 23 mm and 43 mm. It will be appreciated that the length of the sheath 704 will also change across this range. In some examples, the sheath 704 may have an inside diameter which may vary by at least about 20 mm.

In other examples, e.g., for a neonatal or pediatric conduit, the sheath may have an inside diameter which may vary between at least about 12 mm and 22 mm (±2 mm). In some examples, the sheath may have an inside diameter which may vary by at least about 10 mm.

In some examples, the number of clockwise braiding elements 904 may be equal to the number of counter-clockwise braiding elements 906. In some examples, the sheath 704 may have between about 75 and 125 braiding elements, or between about 90 and 100 braiding elements. The sheath 704 of the example conduit 700 has about 96 braiding elements in total, made up of 48 clockwise braiding elements 904 and 48 counter-clockwise braiding elements 906. The sheath 704 may be suitable for a conduit 700 intended for use by an adult patient, for example.

In other examples, the sheath may have between about 40 and 60 braiding elements, or between about 45 and 55 braiding elements, e.g., about 48 braiding elements in total, made up of 24 clockwise braiding elements 904 and 24 counter-clockwise braiding elements 906. Such a sheath may be suitable for a conduit for use by a neonatal or pediatric patient, for example.

In some examples, one or more of the braiding elements 904, 906 may have two or more filaments (i.e., the braiding elements may be a multifilament). In other examples, one or more of the braiding elements 904, 906 may be a single filament (monofilament). In some examples, every one of the braiding elements 904, 906 may be a multifilament. In the illustrated example, each of the braiding elements 904, 906 has two adjacent filaments. In another example, e.g., a sheath suitable for a conduit for use by a neonatal or pediatric patient, each of the braiding elements may comprise three adjacent filaments.

In some examples, as shown in FIG. 18, the filaments 1802 may be interleaved in a regular braid pattern in which a pair of side-by-side filaments 1802 pass under two pairs of side-by-side filaments 1802, then over two pairs of side-by-side filaments 1802. In other examples, the filaments 1802 may be interleaved in a regular braid pattern in which a single filament 1802 passes under two single filaments 1802 then over two single filaments 1802. In other examples, the filaments 1802 may be interleaved in a diamond braid pattern in which a pair of side-by-side filaments 1802 pass under a pair of side-by-side filaments 1802, then over a pair of side-by-side filaments 1802. In other examples, the filaments 1802 may be interleaved in a diamond braid pattern in which a single filament 1802 passes under a single filament 1802, then over a single filament 1802.

In some examples, the filaments of each braiding element are untwisted. In other examples, the filaments of each braiding element may be twisted or braided with each other.

The filaments 1802 in the illustrated example may be substantially circular in cross-section. In other examples, the filaments may have a polygonal cross-section, e.g., triangular, square, pentagonal, hexagonal, octagonal, or the like. In other examples, the filaments may have a rounded cross-section, e.g., oval, elliptical, rounded polygonal, or the like.

The filaments 1802 of the example sheath 704 may be formed from polyethylene terephthalate (PET). In other examples, the filaments 1802 may be formed from any one or more of metal, polymer, ceramic and fibrous materials.

One or more of the filaments 1802 and the braiding elements 904, 906, individually or collectively, may be substantially inextensible at the loads they will be subjected to in normal use.

In some examples, the number of filaments in the clockwise braiding elements 904 may be equal to the number of filaments in the counter-clockwise braiding elements 906. In some examples, the sheath may have between about 150 and 250 filaments, or between about 180 and 200 filaments. The sheath 704 of the example conduit 700 may have about 192 filaments in total.

In other examples, e.g., a sheath suitable for a conduit for use by a neonatal or pediatric patient, the sheath may have between about 120 and 180 filaments, or between about 135 and 165 filaments, e.g., about 144 filaments in total.

In some examples, the filaments may all have the same diameter. In other examples, the filaments may have two or more different diameters.

In some examples, the filaments may have a diameter of between about 0.1 mm and 0.4 mm, or between about 0.2 mm and 0.3 mm. The sheath 704 of the example conduit 700 may have filaments with a diameter of about 0.25 mm.

FIG. 19 illustrate the sheath 704 in a conditioned state.

As the sheath 704 expands and contracts in the longitudinal direction and radial direction, the openings 908 may undergo an affine transformation. That is, the shape of the openings 908 changes. A pitch of the braiding elements 904, 906 (that is, the angle of the braiding elements and thus the number of helical windings about the elongate tube 702 per unit length thereof) may vary.

In this equilibrated state the openings 908 may be relatively elongated in the tangential direction as compared to the longitudinal direction.

When compared with the equilibrated state shown in FIG. 18, it can be observed that the openings 908 in FIG. 19 are relatively shorter in the longitudinal direction. And relatively longer in a tangential direction.

The length and diameter of the sheath 704 may be inversely related. Different braided tubular meshes may be characterized by the gradient of their length vs diameter (L/D) relationship.

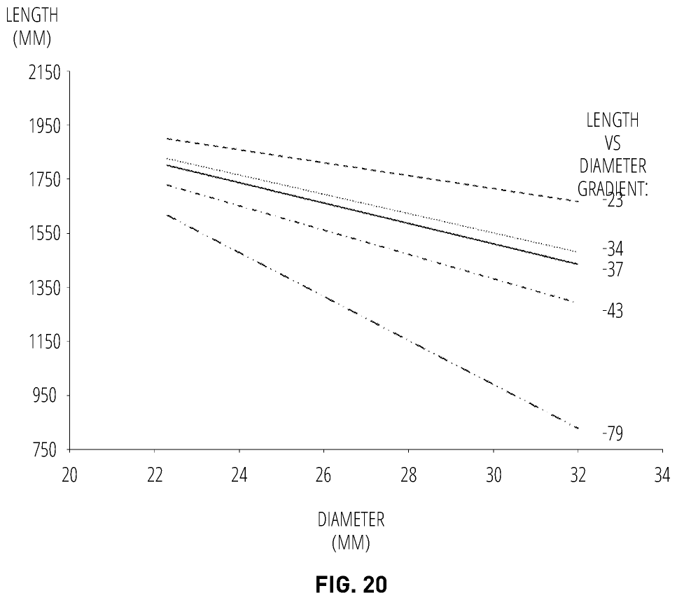

FIG. 20 illustrates a graph of the length vs diameter gradient of a number of examples different braided tubular meshes.

The characterization process was carried out by cutting a sample specimen of a braided tubular mesh sheathing to a predetermined length (e.g., 2000 mm) when flattened (as typically stored), then measuring the length of the sample specimen when fitted over, and conforming to, a number of steel rods of suitable known diameters (e.g., diameters of between about 22 mm and 32 mm, or between about 23 mm and 32 mm, for an adult conduit). In some examples, the length vs diameter gradient may be substantially linear within this range. A different initial length and diameter may yield a similar characteristic response dependent on the configuration of the braiding elements. A length vs diameter gradient can be determined by plotting these measurements, and the associated linear trend lines, on a graph. FIG. 20 illustrates sheaths with length vs diameter gradients of about −23, −34, −37, −43 and −79.

A braided tubular mesh having a relatively larger absolute value of length vs diameter gradient will result in a relatively larger reduction in length of the elongate tube as the diameter increases (and vice versa). A smaller absolute value of length vs diameter gradient results in comparatively less change in axial length for the same increase in diameter.

In some examples, e.g., for an adult conduit, the length vs diameter gradient of the sheath 704 of the conduit 700 may be between about −100 and −20, between about −50 and −20, or between about −40 and −35. In one example, the length vs diameter gradient may be about −37.

In other examples, e.g., for a neonatal or pediatric conduit, the length vs diameter gradient of the sheath may be between about −75 and −25, between about −60 and −40, or between about −55 and −45. In one example, the length vs diameter gradient may be about −52.

The spacing 1502 between the elongate tube 702 and the sheath 704 may be created by cutting the selected braided tubular mesh for sheath 704 to a length which is greater than the length of the elongate tube 702 by a predetermined distance when the sheath 704 conforms to the outside of the elongate tube 702 (i.e., the maximum outside diameter of the elongate tube 702 at the peaks of the corrugations). The predetermined distance may be selected so that the final length of the conduit in the conditioned state is similar to the length of the tube in the equilibrated state, as described above. The sheath 704 is axially compressed prior to securing it to the elongate tube 702 so that the respective ends of the elongate tube 702 and the sheath 704 coincide, increasing the average diameter of the sheath 704 to create the spacing 1502. A relatively smaller characteristic length vs diameter gradient may provide one or more of the advantages of minimizing the spacing 1502 which may improve aesthetics, and minimizing the flat length of the braided tubular mesh which may reduce manufacturing costs.

In some examples, the sheath 704 may also mitigate vibration or oscillation of the conduit, in use. The breathable material used for the elongate tube 702 has specific properties that result in dimensional and mechanical changes as water molecules are absorbed into the breathable material and diffuse through the breathable material at a rate dependent on the temperature and humidity boundary conditions on either side of the breathable material. Specifically, the breathable material softens and enlarges upon the uptake of water molecules. When the breathable material is formed into an elongate tube carrying a humid gas, with a humidity level greater than that outside the tube wall, the humidity of the respiratory gases entering the elongate tube may decrease axially as the respiratory gases pass through the elongate tube to the exit as a result of natural diffusion through the breathable material and the resultant temperature drop, both as a result of the heat transfer rate dependent on the boundary conditions across the breathable material and as a result of the diffusion of water molecules into the breathable material whilst the vapor pressure gradient remains positive across the tube wall. This may result in an axial variation in the conduit dimensions and mechanical properties. The breathable material thus has the properties of a functionally graded material (FGM) as water molecules are absorbed into the breathable material and diffuse through it. The sheath diameter and length may be chosen such that the radial expansion and longitudinal expansion may be selectively controlled. At the inlet to the conduit, where high humidity respiratory gases may enter, the uptake of water molecules may be greatest compared to axial positions distal to the inlet. In some examples, the sheath parameters may be chosen to mitigate or minimize expansion of the elongate tube in the longitudinal direction (relative to an equilibrated state) in favor of a radial expansion in the hydraulic diameter of the elongate tube. The uptake of water molecules, in combination with the corrugation profile and the resultant longitudinal constriction of the elongate tube by the sheath, may have the consequence of producing a thicker tube wall section relative to the equilibrated state. Thus the wall thickness of the tube wall may vary axially and this variation may be more pronounced when the sheath is engaged once sufficient dimensional change has occurred. Although the breathable material may soften as a result of the uptake of water molecules, the axial growth restriction resultant from the sheath may result in a reduced separation distance between the corrugation peaks. This reduced separation distance may have a two-fold effect: the wall thickness increases and there is an increased hoop strength. The stiffness of a pipe may depend on the wall thickness, inner surface finish, hydraulic diameter and the material properties. The bending modes of a pipe are excited when the gas velocity exceeds a certain critical value (critical velocity) dependent on the hydraulic diameter and the inner surface roughness relative to the length. By controlling the longitudinal and radial expansion of the elongate tube, the sheath therefore may indirectly influence the critical velocity relative to a pipe of uniform hydraulic diameter and uniform wall thickness. Specifically, the critical velocity of the respiratory gases may be increased, potentially up to several times that relative to an equivalent pipe of uniform wall thickness and hydraulic diameter. Therefore, for the operating length of the elongate tube when passing humid respiratory gases at typical maximum velocities appropriate for the therapy, the sheath may reduce the likelihood of vibration modes along the elongate tube.

### Elongate Tube Expansion in Use

Use and behaviour of the example conduit 700 will be described below by way of example with reference to FIG. 21 to FIG. 26. It is to be appreciated that this description relates to a particular example only, and the structure and arrangement of conduits in other examples may be selected to behave differently. In other examples the conduit may be configured and arranged to alternatively expand primarily in the longitudinal direction, or expansion may be limited to only one of the longitudinal direction or the radial direction by selecting a sheath with the appropriate properties, e.g., braid pattern, length vs diameter gradient, and the like.

It is to be appreciated that FIG. 21 is not shown to scale and changes in dimensions and proportions between the different states may be exaggerated for illustrative purposes. FIG. 21 also illustrates a substantially uniform expansion of the elongate tube 702, aside from tapering towards the connectors 706. As described elsewhere, expansion may not necessarily be uniform in use.

FIG. 21(a) to (d) (on the left hand side of FIG. 21) schematically illustrate an elongate tube 702 and a pair of connectors 706 without the sheath 704 (referred to as an unsheathed conduit 2108), in various different states.

FIG. 21(e) to (h) (on the right hand side of FIG. 21) schematically illustrate the previously-described example conduit 700, including the sheath 704, in various different states.

FIGS. 21(a) and (e) illustrate the unsheathed conduit 2108 and the conduit 700, respectively, in the equilibrated state, unpressurized.

FIGS. 21(b) and (f) illustrate the unsheathed conduit 2108 and the conduit 700, respectively, in a partially conditioned state, unpressurized. It can be observed that both the unsheathed conduit 2108 and the conduit 700 have expanded in both the radial direction and longitudinal direction. But the conduit 700 less so than the unsheathed conduit 2108.

FIGS. 21(c) and (g) illustrate the unsheathed conduit 2108 and the conduit 700, respectively, in a conditioned state after prolonged use, unpressurized. It can be observed that the unsheathed conduit 2108 has further expanded in both the radial direction and longitudinal direction. Whereas the conduit 700 has further expanded in the radial direction, but contracted in the longitudinal direction. The conduit 700 may return to, or near, its original length.

FIGS. 21(d) and (h) illustrate the unsheathed conduit 2108 and the conduit 700, respectively, in the same conditioned state after prolonged use, but this time pressurized. It can be observed that the unsheathed conduit 2108 has further expanded in both the radial direction and the longitudinal direction. But the conduit 700 remains unchanged.

Referring to FIGS. 21(a) and (e), when respiratory treatment of a patient is to commence, a new breathing circuit 142 may be set up with the gases source 102. The conduit 700 may be removed from packaging in which the gases within the lumen 1202 have generally the same temperature and humidity as the gases outside the tube wall of the elongate tube 702 within the packaging. That is, there is no vapor pressure differential to drive transmission of water molecules through the tube wall of the elongate tube 702. The gases within the packaging may have a similar temperature and relative humidity to ambient air, and the conduit 700 may therefore be in an equilibrated or near-equilibrated state when removed from the packaging. In some cases, the conduit 700 may be left for a period after removal from the packaging and before use (e.g., stored ready for use), with ambient air filling the lumen of the elongate tube 702. If the humidity of the ambient air differs from that within the packaging, the elongate tube 702 may absorb or desorb water molecules while it comes to an equilibrated state with the ambient air. The unsheathed conduit 2108 and the conduit 700 are shown in this equilibrated state in FIG. 21(a) and FIG. 21(e), respectively. In this state, there is a spacing 1502 between the elongate tube 702 and the sheath 704 of conduit 700, as described above.

The sheath 704 may be configured to be at least partially spaced from the elongate tube 702 in this equilibrated state. This spacing may mitigate further stiffening of the conduit 700 in the equilibrated state. An overly stiff conduit 700 may be problematic for usability. For example, it may increase tube drag forces on the Y-piece 110 or patient interface 112. In some examples, the sheath 704 in this equilibrated state may make no, or no more than negligible, contribution to the stiffness of the conduit 700.

As illustrated in FIG. 21(e), the sheath 704 at its respective ends may taper towards the respective connector 706 in at least the equilibrated state.

It has been found that the breathable material of the elongate tube 702 may be sufficiently stiff for the conduit 700 to meet at least one or more of the compliance and resistance to flow requirements of the ISO 5367:2014(E) standard, when in the equilibrated state or when conditioned as set out in the standard. But the conduit 700 may not necessarily meet one or more of these requirements in the conditioned state as defined herein.

Once the conduit 700 is connected to the respiratory assistance system and therapy commences, the lumen of the elongate tube 702 is exposed to a flow of respiratory gases with relative humidity which may be upwards of 90%, and often about 100%, at the patient end. The ambient air may have a relative humidity of between about 10% and 90%, and often within the ranges of about 40% to 60% or 45% to 55% which are suggested for everyday health and comfort. There is a differential in the partial pressure of water vapor between the respiratory gases within the lumen 1202 and the ambient air outside the elongate tube 702. The solution-diffusion mechanism causes the breathable material of the elongate tube 702 to continuously absorb water molecules from the respiratory gases and condensate (if any) or other liquids (if any) within the lumen, and desorb the water molecules to the ambient air outside the elongate tube 702 according to the driving gradient.

As described in further detail below with reference to FIG. 27, expansion of the elongate tube 702 in a conditioned state, in use, may not necessarily be uniform along the length of the elongate tube 702. But for the purposes of explanation, the following description assumes that changes in dimensions of the elongate tube 702 and the sheath 704 occur generally uniformly.

The initial absorption of water molecules from within the lumen causes the elongate tubes 702 of the unsheathed conduit 2108 and the conduit 700 to tend to expand in both the longitudinal direction 902 and the radial direction 1504 (as well as in wall thickness).

In the case of unsheathed conduit 2108, this expansion is unconstrained as illustrated in FIG. 21(b).

In the case of the conduit 700, the spacing 1502 between the elongate tube 702 and the sheath 704 means that the elongate tube 702 may expand somewhat in both the longitudinal direction and the radial direction. Expansion of the elongate tube 702 in the longitudinal direction will cause a corresponding elongation of the sheath 704. Elongation of the elongate tube 702 and the sheath 704 may, in turn, cause the sheath 704 to contract in the radial direction.

As shown in FIGS. 21(c) and (g), prolonged use of the conduit 700 and exposure to the humidity differential (i.e., further absorption of water molecules) may cause the elongate tube 702 to engage the inner surface of the sheath 704. The spacing 1502 between the elongate tube 702 and the sheath 704 may be eliminated along at least a portion of the length of the elongate tube 702. Further expansion of the elongate tube 702 in at least one of the radial direction and the longitudinal direction may be constrained by the sheath 704. That is, each of the braiding elements of the sheath 704 are in tension and the sheath 704 has a hoop stress due to expansion of, and engagement with, the elongate tube 702.

As the elongate tube 702 continues to absorb water molecules from within the lumen, some further radial expansion of the elongate tube 702 may occur. This may cause the sheath 704 to contract in the longitudinal direction. The conduit 700 may return to, or near (e.g., between about 90% and 110%), its original length (in the equilibrated state).

As shown in FIG. 21(d), pressurization of the unsheathed conduit 2108 may cause further expansion in both the radial direction and the longitudinal direction. This may be in part due to the softening of the breathable material due to absorption of a relatively large mass of water molecules. But the sheath 704 constrains further expansion of the conduit 700, as illustrated in FIG. 21(h).

As the length of the elongate tube 702 varies in use, as described above, a corrugation pitch (that is, a distance between adjacent peaks of the corrugations in the elongate tube 702) may also vary. But in the conduit 700, this variation is constrained by the sheath 704 as is apparent from a comparison between FIG. 21(b) (unsheathed) and FIG. 21(f) (sheathed), for example. In cases where the interaction between the elongate tube 702 and the sheath 704 causes the elongate tube 702 to initially expand in the longitudinal direction and then contract to return to, or near, its original length, it will be appreciated that the corrugation pitch of the elongate tube 702 will similarly increase and then decrease to return to, or near, the original corrugation pitch.

As described elsewhere, the elongate tube 702 may not necessarily absorb water molecules uniformly along the length of the corrugated portion 1016. In particular, it will be appreciated that the respiratory gases in the lumen may have a higher absolute humidity at the patient end of the conduit due to the dehumidifying effect of the breathable material upon the respiratory gases as they pass along the length of the elongate tube 702 towards the gases return inlet 140. Moreover, gravity will cause any condensate which does form within the lumen to drain towards, and accumulate at, the lowest portion of one or more of the annular corrugations 2202 and the elongate tube 702 (as a whole). This may result in the breathable material of the elongate tube 702 absorbing a higher concentration of water molecules in a first region of the elongate tube 702 when compared to a second region of the elongate tube 702. This in turn may result in localized expansion of the breathable material in the first region, in one or more of the radial direction and the longitudinal direction. And a pitch of the corrugations 2202 (the number of corrugations per unit length) may vary along the length of the elongate tube 702.

The sheath 704 may permit more expansion of the elongate tube 702 in the first region than it would if the absorption and expansion were uniform along the length of the elongate tube 702. A diameter of the elongate tube 702 in the first region may exceed the average diameter of the elongate tube 702 as a whole. Expansion of the elongate tube 702 in the first region may take up slack in the sheath 704 due to the spacing 1502 in the second region of the elongate tube 702. This localized expansion may increase the surface area and the permeability of the breathable material in the first region of the elongate tube 702, where the increased permeability is most desired (i.e., in the regions of highest concentration of water molecules within the lumen, where condensate is present or most likely to form), relative to the second region.

It is to be appreciated that the behaviour of conduits according to the present disclosure may be modified or tuned by varying a number of factors to meet particular design requirements. A number of factors have been found to affect the behaviour of conduits according to this disclosure, including at least the:
i. length/diameter (L/D) characteristic of the sheath,
ii. profile and pitch of the corrugations of the elongate tube, and
iii. spacing between the elongate tube and the sheath.

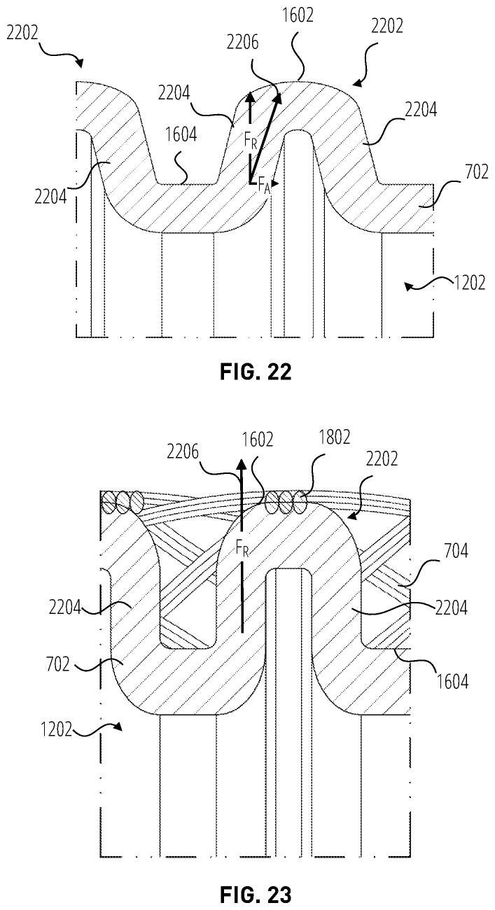

FIG. 22 illustrates an example profile of corrugations 2202 of an elongate tube 702 in an equilibrated state, in cross-section. The sheath 704 is omitted from this drawing. The drawing depicts only an upper portion of a length of the elongate tube 702 in a horizontal orientation, with the lumen 1202 towards the lower part of the drawing and ambient air at the upper part of the drawing.

Each of the corrugations 2202 have side walls 2204 which converge toward the peak 1602 of the corrugation 2202 (that is, defining the maximum outside diameter of the corrugation 2202 and the elongate tube 702) when the elongate tube 702 is equilibrated.

The peak 1602 may be substantially rounded. A trough 1604 of each of the corrugations 2202 may be substantially flattened. That is, each trough region may be substantially cylindrical (at least when the elongate tube 702 is arranged in a linear configuration).

FIG. 23 illustrates a profile of the corrugations 2202 of FIG. 22 in cross-section when the elongate tube 702 is in a conditioned state, and more particularly a conditioned state in which expansion of the elongate tube 702 is constrained by the sheath 704.

As the material of the elongate tube 702 expands due to absorption of water molecules, the sheath 704 constrains expansion of the elongate tube 702 in the longitudinal direction, as described above. The expansion of the elongate tube 702 against the constraint of the sheath 704 may cause the corrugations 2202 to compress closer together in the longitudinal direction. This in turn may cause the profile of the corrugations 2202 to change from the cross-section shown in FIG. 22 to the cross-section shown in FIG. 23. That is, as the elongate tube 702 expands against the constraint of the sheath 704, the side walls may reorient towards, and potentially beyond, the radial direction (i.e., the vertical direction, as illustrated). The side walls may reorient from forming an acute angle with the longitudinal direction, towards a radial direction (90° with respect to the longitudinal direction), and possibly even beyond the radial direction to form an obtuse angle with the longitudinal direction.

In the particular conditioned state illustrated in FIG. 23, each of the side walls 2204 have a generally radial orientation (extending vertically in this cross-sectional drawing, 90° with respect to the longitudinal direction), whereby the side walls 2204 are substantially planar and adjacent side walls 2204 are generally parallel to each other (at least when the elongate tube 702 is arranged in a linear configuration).

The peak 1602 of the corrugation 2202 joining the adjacent side walls 2204 may also become relatively flattened when compared to the initial profile of FIG. 22. That is, the peak of the corrugation 2202 may become substantially cylindrical as shown in FIG. 23. Whereas in the equilibrated state the peak may curve continuously intermediate the side walls 2204 as shown in FIG. 22.

The radial orientation of the side walls 2204 in FIG. 23 means that the force vector 2206, equivalent in this example to the radial force component FR, opposes any radially inward crushing force. The crushing force may be due to the weight of a patient's arm lying upon the conduit, for example. An unsheathed conduit 2108, on the other hand, may tend to maintain a corrugation profile generally similar to that of FIG. 22 as it expands. The force vector 2206 in FIG. 22 includes both a radial force component FR in the radial direction and an axial force component FA in the longitudinal direction. The radial force component FR may be relatively smaller than in FIG. 23.

The corrugations may tend to compress closer together with expansion of the material, due to the axial constraint of the sheath 704 upon the elongate tube 702. This axial “spring compression force” may follow Hooke's Law, stiffening the elongate tube 702 and acting against the atmospheric pressure axially compressing the elongate tube 702 as the internal pressure decreases.

The longitudinal constraint imposed by the sheath 704 may cause corrugation growth, due to absorption of water molecules, to occur into gaps between the corrugations 2202 (i.e., thicker corrugations). The radial constraint may cause corrugation growth radially inwards (towards the center of the elongate tube 702). In some examples, the gaps between the corrugations 2202 may partially or entirely close, potentially resulting in effectively a single thick wall section without any pivot points or moment effect from the corrugation radii.

One or more of these effects, resulting from interaction between the elongate tube 702 and the sheath 704 as the elongate tube 702 expands due to absorption of water molecules, may mitigate softening of the breathable material. This may enable the conduit 700 to comply with one or more of the relevant standards and other self-imposed design requirements throughout a range of different operating conditions. For example, in testing in a conditioned state it has been found that a particular example unsheathed conduit 2108 had a compliance (defined in the Glossary, below) exceeding 4 milliliters per centimeter of water (ml/cmH₂O) or 5 ml/cmH₂O. In some examples, the conduit 700 of the present disclosure with the sheath 704, in a conditioned state as defined herein, may have a compliance of less than about 4 ml/cmH₂O, less than about 2.5 ml/cmH₂O, or less than about 1.2 ml/cmH₂O, when otherwise tested in accordance with the ISO 5367:2014(E) standard.

In other examples, the compliance standard may be met throughout a range or sub-range of anticipated operating conditions (conditioned states) by selecting a breathable material or otherwise configuring the elongate tube to absorb a relatively lower proportion of water molecules, e.g., between about 40% and 60% of its dry mass.

FIG. 23 also illustrates an alternative sheath 704 in which each braiding element has three filaments 1802 arranged side-by-side.

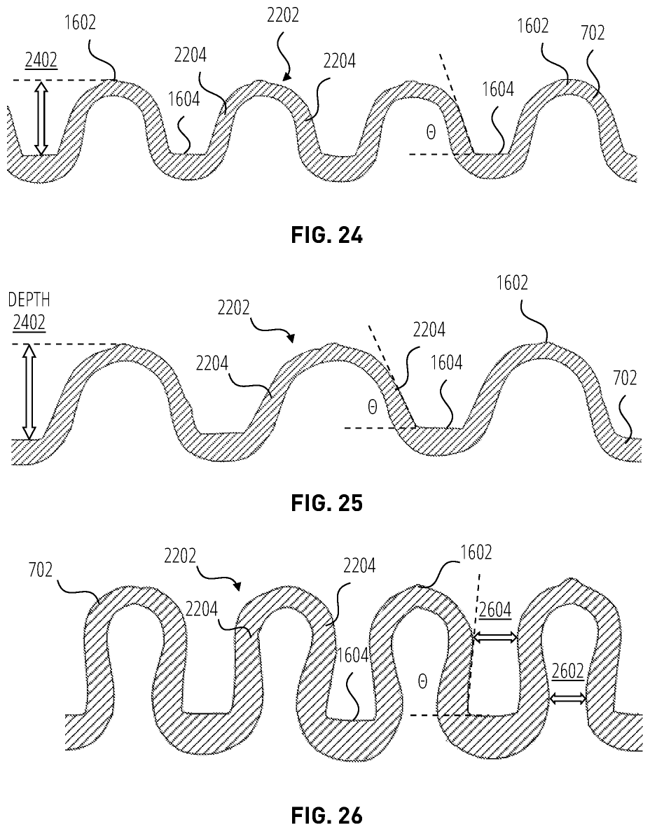

FIG. 24 illustrates a cross-section of a portion of an elongate tube 702 of a conduit 700 when in an equilibrated state, in which the elongate tube 702 is not constrained by the sheath 704 (not shown in this drawing).

The side walls 2204 of each corrugation 2202 generally converge towards the peak 1602 of the corrugation 2202. In some examples, the side walls 2204 may be configured to, in this equilibrated state, extend at an angle θ from the adjacent trough of between about 65° and 85°, or between about 70° and 80°, with respect to the longitudinal direction (the horizontal direction, as illustrated in this drawing). In the example elongate tube 702 depicted in FIG. 24, the side walls 2204 may extend at an angle θ of about 75° in the equilibrated state.

In FIG. 24, it can be observed that the troughs are relatively flattened. In cross-section with the elongate tube 702 arranged substantially linearly between adjacent side walls 2204, as shown, the outer surface of the trough may be substantially linear. The outer surface of the elongate tube 702 at the troughs may be substantially cylindrical. By contrast, the peaks 1602 may curve substantially continuously between adjacent side walls 2204.

FIG. 25 is a cross-section of the elongate tube 702 in a conditioned state, in the absence of a sheath 704 constraining expansion of the elongate tube 702. FIG. 25 is depicted at about the same scale as FIG. 24.

It can be observed that there are fewer corrugations 2202 (about 3 corrugations 2202 in FIG. 25, compared to about 4 corrugations 2202 in FIG. 24) for a given length of the elongate tube 702, due to unconstrained expansion of the elongate tube 702 in the longitudinal direction (the horizontal direction, as shown in this drawing) upon absorption of water molecules. The side walls 2204 of each corrugation 2202 still generally converge towards the peak of the corrugation at about the same angle θ as in the equilibrated state of FIG. 24. That is, the side walls 2204 of the example elongate tube 702 of FIG. 25 were found to extend at an angle θ of about 75° in the conditioned state, in the absence of the sheath.

It can also be observed that a depth 2402 of the corrugations, measured from the peak 1602 to an adjacent trough 1604 of a corrugation 2202 at an external surface of the elongate tube 702, is about 33% greater in the conditioned state depicted in FIG. 25 due to unconstrained expansion of the elongate tube 702.

FIG. 26 is a cross-section of the elongate tube 702 in a conditioned state, this time constrained by a sheath 704 (not shown). FIG. 26 is depicted at about the same scale as FIG. 24 and FIG. 25.

It can be observed that a pitch of the corrugations is about the same as in the equilibrated state of FIG. 24. That is, the elongate tube 702 has about four corrugations 2202 in the illustrated length of the elongate tube 702.

In this constrained conditioned state, the side walls 2204 of the corrugations 2202 do not converge continuously from the trough to the peak of the corrugation 2202. In some examples, the side walls 2204 may form an angle θ of greater than 85°, about 90°, or greater than 90° (obtuse). That is, from the adjacent troughs 1604 towards an intermediate peak 1602, the side walls 2204 of a corrugation first diverge and then converge towards the peak 1602.

The side walls 2204 may reorient towards the radial direction (90°) from the initial angle (75° in this example), and in some cases beyond the radial direction (forming an obtuse angle θ as shown in FIG. 26), as the elongate tube 702 absorbs an increasing mass of water molecules.

In the example elongate tube 702 depicted in FIG. 26, the side walls 2204 were found to extend at an angle θ of about 95°. There may be a point of inflection within each of the side walls 2204, as well as at the peak and the trough of each corrugation 2202.

A profile of each of the corrugations 2202 in FIG. 26 is substantially Omega (Ω)-shaped, whereas in FIGS. 21 and 22 the corrugations 2202 are generally more sinusoidal in cross-section (albeit with flattened troughs, in this example). That is, in FIG. 26 the corrugations double back on each other, whereas in FIGS. 21 and 22 they do not. This may form interior narrowed waist regions 2602 and exterior narrowed waist regions 2604 between facing side walls 2204 of adjacent corrugations 2202, intermediate the respective peaks and troughs.

### Non-Uniform Expansion

FIG. 27 illustrates a conduit 700 according to the present disclosure after prolonged use as the expiratory conduit 114 of an example respiratory assistance system 2700. The drawing is not necessarily to scale. In particular, expansion of the elongate tube 702 or the sheath 704 may be exaggerated for illustrative purposes. And the inspiratory conduit 108 and expiratory conduit 114 are not shown to scale.

Except as apparent from the drawing and the description below, the respiratory assistance system 2700 may be similar to the respiratory assistance system 100.

FIG. 27 illustrates the filter 2702 at the gases return inlet 140 of the gases source 102. The filter 2702 may be connected directly between the expiratory conduit 114 and the gases return inlet of the gases source 102.

The humidifier 106 in this example may be an F&P 820™ Heated Humidifier also available from Fisher & Paykel Healthcare Limited.

The respiratory assistance system 2700 in this example is shown in use, after a prolonged period of delivering humidified respiratory gases to a patient (not shown). It is to be appreciated that this represents merely one of a number of different possible conditioned states which will depend upon a number of variables including, but not limited to: time of use, temperature of the respiratory gases, humidity of the respiratory gases, temperature of the ambient air, humidity of the ambient air, routing of the conduit, type and model of gases source, patient condition and humidity contribution, nebulized substances, and any breeze upon the conduit, for example.

The expiratory branch 146, and in particular the expiratory conduit 114, in this example omits both a heater wire and a water trap.

As illustrated, the expiratory conduit 114, or more particularly the elongate tube 702 and the sheath 704, are flexible in at least this conditioned state. The expiratory conduit 114 may also be flexible (albeit possibly to different degrees) in any one or more of the dry state, the equilibrated state and the saturated state. Because it is flexible, the expiratory conduit 114 may adopt a curvilinear shape in use, as shown.

It has been found that, in use, the elongate tube 702 may not necessarily expand uniformly along its length, between the connectors 706. That is, the diameter of the elongate tube 702 may not be necessarily uniform along its length in a conditioned state. This may be because the concentration of water molecules within the lumen may vary along the length of the elongate tube 702. This variation may be due to variations in one or more of the humidity of the respiratory gases (i.e., water vapor) and accumulation of condensate (i.e., liquid water) along the length of the elongate tube 702. The partial pressure of water vapor in ambient air outside the elongate tube 702, on the other hand, will be relatively constant along the length of the elongate tube 702.

In this example conditioned state, localized expansion of the elongate tube 702 can be observed in three distinct regions of the expiratory conduit 114—an inlet region 2704, an outlet region 2706, and an intermediate region 2708. In other examples, there may be two or more intermediate regions 2708. In yet other examples, the elongate tube 702 may have localized expansion in any one or more of the inlet region 2704, the outlet region 2706, and one or more intermediate regions 2708. In yet other examples, there may be no distinct localized expansion.

The regions of localized expansion may be portions of the length of the elongate tube 702 which have a larger diameter relative to an adjacent region (or regions) of the elongate tube 702. In these regions, the elongate tube 702 may have a bulged appearance. The bulge may be tapered. In some examples, the localized expansion may be readily apparent to the naked eye. In some examples, the region of localized expansion may have a maximum diameter which is at least about 5%, at least about 10%, at least about 20%, or at least about 30% greater than in an adjacent region (or regions) of the elongate tube 702 (e.g., for the outlet region 2706, the region intermediate the outlet region 2706 and the intermediate region 2708).

It is to be appreciated that the term “localized expansion” is not intended to mean that expansion is limited to these regions. The term is used in the relative sense. That is, expansion may be more pronounced in the regions of localized expansion than in an adjacent region.

In describing the inlet region 2704, outlet region 2706 and intermediate region 2708 below, references to proportions of the length of the elongate tube 702 refer to the length of the elongate tube 702 between the connectors 706, e.g., excluding the cuff portions 1002 within the connectors 706. It will be appreciated that the sum of these lengths will not exceed 100% of the length of the elongate tube 702 between the connectors 706. In some examples, the sum of the lengths of the inlet region 2704, outlet region 2706 and intermediate region 2708 may be less than about 75%, less than about 50%, or less than about 33% of the overall length of the elongate tube 702 between the connectors 706.

The inlet region 2704 may be within, or correspond to, a portion of the length of the elongate tube 702 which is nearest one or more of the Y-piece 110, the patient interface (not shown) and the patient (not shown). In some examples, the inlet region 2704 may be up to about 50%, up to about 33%, up to about 25%, up to about 20%, or up to about 10% of the length of the elongate tube.

It has been found that at least one of the relative humidity of the respiratory gases and the volume of condensate within the lumen of the elongate tube 702 may be elevated in the inlet region 2704, relative to one or more other regions of the elongate tube 702. For example, relative to a region intermediate the inlet region 2704 and the intermediate region 2708, or relative to a region intermediate the intermediate region 2708 and the outlet region 2706. The elevated relative humidity may be due to the dehumidifying effect of the breathable material as the respiratory gases pass along the length of the lumen, for example. The elevated volume of condensate may be due to condensate draining into the inlet region 2704 from the Y-piece 110 or further upstream from the Y-piece, e.g., from a catheter mount, a patient interface and a patient (none of which are shown in FIG. 27), for example. At least some of this condensate may accumulate within the corrugations of the elongate tube 702 in the inlet region 2704.

The outlet region 2706 may be within, or correspond to, a portion of the length of the elongate tube 702 which is nearest one or more of the filter 2702, gases return inlet 140 and gases source 102. In some examples, the outlet region 2706 may be up to about 50%, up to about 33%, up to about 25%, up to about 20%, or up to about 10% of the length of the elongate tube.

It has been found that at least one of the relative humidity of the respiratory gases and the volume of condensate within the lumen of the elongate tube 702 may be elevated in the outlet region 2706, relative to one or more other regions of the elongate tube 702. For example, relative to a region intermediate the inlet region 2704 and the intermediate region 2708, or relative to a region intermediate the intermediate region 2708 and the outlet region 2706. The elevated relative humidity or volume of condensate may be due to condensate draining into the outlet region 2706 from the filter 2702 or the gases source 102, for example.

The intermediate region 2708 may be within a portion of the length of the elongate tube 702 which is intermediate the inlet region 2704 and the outlet region 2706. The intermediate region 2708 may be up to about 50%, up to about 33%, up to about 25%, up to about 20%, or up to about 10% of the length of the elongate tube.

It has been found that at least one of the relative humidity of the respiratory gases and the volume of condensate within the lumen of the elongate tube 702 may be elevated in the intermediate region 2708, relative to one or more other regions of the elongate tube 702. For example, relative to a region intermediate the inlet region 2704 and the intermediate region 2708, or relative to a region intermediate the intermediate region 2708 and the outlet region 2706.

The flexibility of the expiratory conduit 114 means that the expiratory conduit 114 may tend to drape between the Y-piece 110 and the gases source 102. This drape may result in at least one low point 2710 intermediate the Y-piece 110 and the gases source 102. The low point 2710 may be at a height below the ends of the expiratory conduit 114 (e.g., below the Y-piece 110 and the gases return inlet 140). Any condensate within the lumen 1202 may tend to drain towards, and accumulate at, the low point 2710 due to gravity. This condensate may have formed in the elongate tube 702, or may have been contributed to the conduit from other components of the respiratory assistance system 2700 (such as a catheter mount, Y-piece, connectors, or patient). The elevated relative humidity or volume of condensate in the intermediate region 2708 may be due to condensate accumulating at the low point 2710.

In some examples, it has been found that, in use, the conduit 700 may first exhibit localized expansion in the inlet region 2704. Localized expansion in one or more of the outlet region 2706 and the intermediate region 2708 may follow later, e.g., as condensate begins to form in the filter 2702 or the gases source 102 and drain into the conduit 700. In some applications, periodic replacement of the filter 2702 may mitigate condensation in the filter and localized expansion in one or more of the outlet region 2706 and the intermediate region 2708.

Similarly, expansion of the elongate tube 702 may not necessarily be uniform in one or more of the circumference and wall thickness of the tube wall, e.g., in a cross-section perpendicular to the longitudinal direction. For example, any condensate in the elongate tube 702 may accumulate at a lower region of an annular corrugation (i.e., the peaks 1602 when viewed from outside the elongate tube 702) due to gravity. The elongate tube 702 may expand more in this lower region compared to an upper region of the corrugation. It should be appreciated that the lowest point of a corrugation in real space is actually a corrugation “peak” when viewed in cross-section, or from outside the elongate tube.

It has been found that expansion of the breathable material may increase its permeability to water molecules, when compared to a constrained breathable material under the same conditions. This may be due to the relatively greater surface area of the expanded breathable material.

Non-uniform expansion of the elongate tube 702 may advantageously increase the elongate tube's permeability to water molecules where it is most needed. For example, one or more of the inlet region 2704, outlet region 2706 and the intermediate region 2708. The elongate tube 702 may automatically adapt to different or changing operating conditions, in use. By contrast, a water trap is provided in a fixed location along the length of the conduit, e.g., a mid-point. As shown in FIG. 27, the mid-point or other fixed location may not necessarily coincide with the low point 2710 of the conduit, significantly limiting its effectiveness.

### Manufacture

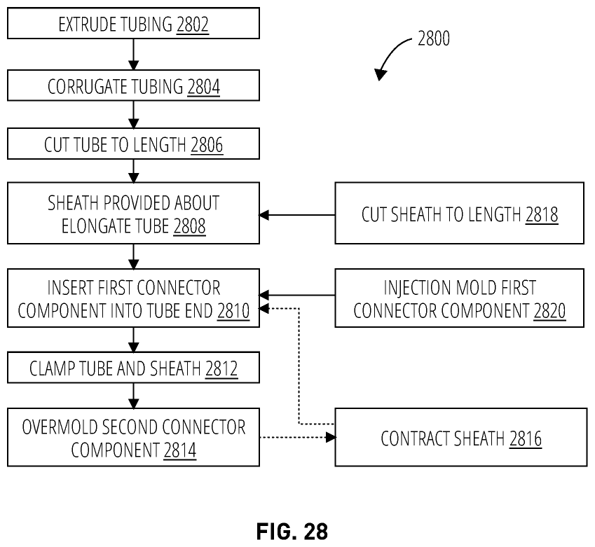

FIG. 28 is a flowchart illustrating an example method 2800 for manufacturing a conduit 700 in accordance with the present disclosure.

At step 2802, a continuous length of tubing suitable for use as elongate tube 702 may be produced by extruding the breathable material.

At step 2804, the extruded tubing may be at least partially corrugated by passing the semi-molten extruded tubing through a corrugator block. In some examples, portions of the extruded tubing may be left uncorrugated. These portions may become the uncorrugated cuff portions 1002. Alternatively, the entirety of the extruded tubing may be corrugated.

At step 2806, the elongate tube 702 may be formed by cutting a length from the extruded and corrugated tubing. In some examples, the extruded and corrugated tubing may be cut at an uncorrugated portion. The cut may be made substantially centrally in the uncorrugated portion.

At step 2808, the sheath 704 may be provided about the elongate tube 702. The elongate tube 702 may be inserted into the sheath 704. In some examples, the sheath 704 may have been previously cut to a predetermined length at step 2818. The sheath 704 may be cut from a roll of flattened braided tubular mesh sheathing. The sheath 704 may be cut to a length which is longer than the elongate tube 702 when deformed to conform to the outside of the elongate tube 702, as described above.

At step 2810, the first connector component 1004 may be inserted into a first end of the elongate tube 702. In some examples, the first connector component 1004 may have been previously injection molded at step 2820.

At step 2812, the first connector component 1004, the first end of the elongate tube 702 and a corresponding first end of the sheath 704 may be clamped together within a mold tool. The mold tool may have protrusions to hold the first end of the sheath 704 so that it overlies the first end of the elongate tube 702. In some examples, the mold tool may have opposing pairs of protrusions.

At step 2814, a molten plastic material may be injected into the mold tool to form the second connector component 1006. That is, the second connector component 1006 may be overmolded onto the first connector component 1004, the first end of the elongate tube 702, and the first end of the sheath 704, permanently securing the components together. The apertures 1014 are formed by the molten plastic material flowing around the protrusions which clamp the elongate tube 702 and sheath 704 in position. Once the molten plastic material cools and solidifies, the connector 706 is complete and may be ejected from the mold.

Steps 2810 to 2814 may be repeated, or those same steps may be duplicated simultaneously, to form the second connector 706 at the opposing second end of the conduit 700. The sheath 704 may first be contracted in the longitudinal direction at step 2816 so that its ends coincide with, and overlie, corresponding ends of the elongate tube 702, creating the spacing 1502. In some examples, the connectors 706 of the conduit 700 may be identical. In other examples the connectors at opposing ends of the conduit may differ from each other. In some examples, only one of the first connector component 1004 and the second connector component 1006 might differ between the two connectors 706. In other examples, they both might differ.

In other examples, the second connector component 1006 may be injection molded separately. The second connector component 1006 may be attached to the first connector component 1004 by welding, adhesive, or interference fit (e.g., snap-fit), for example.

### Glossary

“Breathable material” refers to a non-porous permeable material that allows the passage of water molecules through a monolithic wall of the permeable material via the solution-diffusion mechanism, without allowing the bulk passage of liquid water or bulk flow of respiratory gases all the way through the wall. It should be appreciated by one of skill in the art that the water molecules in the wall are molecularly dispersed in the media, and are therefore without a state (solid, liquid, or gas), although they are sometimes referred to in the art as vapor (e.g., the rate of transfer is often referred to as a moisture vapor transmission rate (MVTR) or the like). It should further be appreciated that a monolithic wall does not contain open channels or pores from one major surface to another, such that pathogens could be carried through such channels alongside air or liquid water drops via the pore flow mechanism. However, this definition is not intended to exclude a tube formed from such a breathable material which may have one or more holes provided through the material, such as might arise from a manufacturing defect for example, which may result in negligible pore flow which does not materially affect the overall performance of the tube and compliance with the leakage requirements of ISO 5367:2014. It should yet further be appreciated that, like all polymers, some small molecule transport of respiratory gases (such as oxygen, carbon dioxide, nitrogen or helium) may occur in trace or de minimis amounts (i.e., not “bulk” flow), which, for a breathable material as defined herein, would typically be at a rate at least an order of magnitude lower than that for water molecules. Furthermore, of particular relevance for respiratory gases being delivered to or from a patient, such small molecule transport of respiratory gases would be of an amount less than that allowed for compliance with the relevant standards, for example, in the leakage test of ISO 5367:2014, which is hereby incorporated by reference in its entirety, at Section 5.4 tested via the method set out in Annex E.

“Compliance” is defined by ISO standard 4135:2001, which is hereby incorporated by reference in its entirety, at Section 3.1.5 as the “volume added per unit pressure increase when gas is added to an enclosed space, expressed at the temperature and humidity of that enclosed space at ambient atmospheric pressure” (@) ISO 2001). The method for testing compliance of a conduit according to the present disclosure is based upon the method set out in ISO 5367:2014, which is incorporated by reference in its entirety, at Annex H. First, any leaks in the conduit equal to, or greater than, 1 ml/min are sealed (as described in Annex E). Second, the conduit is conditioned at 42±3° C. at not less than 80% relative humidity for at least one hour. Third, one end of the conduit is blocked off and the conduit is placed on a flat surface. Fourth, a pressure measuring device is connected to the opposing end of the conduit. Fifth, the conduit is inflated to a stable gauge pressure of 60±3 cmH₂O over a period of five seconds or less, and the volume of air required is recorded. It will be appreciated that the conditioning defined by the standard may not reflect in use conditions and the standard was not drafted with breathable materials in mind. To better reflect in use conditions, compliance of a conduit formed at least in part from a breathable material may be further tested by additionally, or alternatively, conditioning the conduit to the simulated conditioned state as described below.

“Conditioned” refers to one of a continuum of states in which an elongate tube has been exposed to a water vapor pressure gradient, with a relatively higher partial pressure of water vapor within the lumen (i.e., higher than the partial pressure of water vapor of the ambient air), for a prolonged period of time. That is, the tube wall of the elongate tube has absorbed water molecules from within the lumen, and may be continuing to absorb water molecules from within the lumen. In particular, a conduit which has been, and continues to be, used to convey a humidified flow of respiratory gases for a period of time may be said to be in a conditioned state. The tube wall of an elongate tube in the conditioned state will therefore generally contain a higher concentration of water molecules than in the dry or equilibrated states, but generally a lower concentration than in the saturated state. References herein to certain properties of a conduit in “a” (singular) conditioned state are not necessarily intended to apply to all conditioned states, unless otherwise apparent from the context. In particular, depending on design requirements and configuration of the conduit parameters, relative humidity and temperature of the respiratory gases and ambient air, and the concentration of water molecules within the breathable material of the elongate tube, expansion of the elongate tube may or may not be constrained by the sheath in a particular conditioned state. That is, there may be subsets of unconstrained conditioned states and constrained conditioned states within the continuum of conditioned states. A conditioned state may be simulated by conditioning the conduit according to the following method. First, an ambient temperature of 22±2° C. should be reached and maintained throughout the conditioning method. Second, the conduit is laid in a V-tray in an in an equilibrated state. Third, the lumen of the conduit is supplied with a flow of gases at a flow rate of 10 standard liters per minute (SLPM) (ref 20° C., 101.325 kPa), humidified by a humidifier set to a humidity level of 37° C. dew point at 100% relative humidity (RH) for a period of 24 hours.

“Constrain” refers to a non-negligible force exerted by the sheath upon the elongate tube which acts to inhibit expansion of at least a portion of the elongate tube in at least one of the longitudinal direction and radial direction. The effect of the constraint may be localized to a portion of the tube as it transitions from an equilibrated to a conditioned state, or from an unconstrained conditioned state to a constrained conditioned state. Further expansion of an elongate tube constrained by a sheath may be possible, but any such expansion will require expansion forces exceeding the constraining force exerted by the sheath. That is, “constrain” is not intended to necessarily imply that any further expansion is entirely prevented. The related terms “constrained, “constraint,” “constrains” and the like are to be interpreted accordingly. Conversely, “unconstrained” refers to the absence of a force, or the presence of a negligible force, exerted by the sheath upon the elongate tube to resist expansion of the elongate tube. By way of example, the force required to deform a sheath which is not subject to a hoop stress (for example, the sheath is not subject to a hoop stress when the conduit is in the equilibrated state with a spacing between the elongate tube and the sheath) may be regarded as a “negligible force.” Expansion of the elongate tube in the presence of such a negligible force may be regarded as “unconstrained” by the sheath. Or the elongate tube may be said to be able to “freely” expand.

“Differential scanning calorimetry (DSC)” refers to DSC testing for melting point temperatures conducted on test specimens of an elongate tube based on the method set out in ISO standard 11357-3:2018(E) (© ISO, 2018), the entire content of which is hereby incorporated by reference. In a first step, the test specimens are prepared in accordance with ISO 11357-1:2023, the entire content of which is hereby incorporated by reference. Second, the test specimens are dried in accordance with the drying method of ISO 62:2008(E) as briefly described with respect to the second step of the immersion testing method defined below. Third, the apparatus is set up, the test specimen loaded into the crucible, and the crucible inserted in accordance with ISO 11357-1:2023. Fourth, the test specimens are heated from a temperature of about −20° C. to a temperature of about 250° C. at a rate of about 10 degrees Celsius per minute (° C./min). Fifth, the temperature is held for 5 min. Sixth, the test specimens are cooled from about 250° C. to a temperature of about −20° C. at a rate of about −10° C./min. Seventh, the temperature is held for 5 min. Eighth, the test specimens are again heated from a temperature of about −20° C. to a temperature of about 250° C. at a rate of about 10° C./min, while recording the differential heat rate (measured in milli-Watts per milligram, mW/mg) of the sample specimens. The differential heat rate may be plotted against temperature, and the melting point(s) of the test specimens identified from the temperature(s) corresponding to the peak(s) in the plot.

“Dry” refers to a state of the elongate tube, or a sample thereof, which has been dried in accordance with the drying method of ISO 62:2008(E) as briefly described with respect to the second step of the immersion testing method defined below. It is an “artificial” state in that an elongate tube will not generally enter this state during normal use (e.g., in use in an assisted breathing system). The tube wall of an elongate tube in the dry state generally contains a lower concentration of water molecules than in any of the equilibrated, conditioned and saturated states.

“Equilibrated” refers to a state in which an elongate tube, usually free from condensate or other liquid water, has been exposed to ambient air, for example in a controlled environment of 40% to 60% relative humidity, both within the lumen and outside the tube wall, for a period of time sufficient for the elongate tube to reach a steady state. That is, the concentration of water molecules within the breathable material is equilibrated with ambient air. The tube wall of an elongate tube in the equilibrated state will generally contain a higher concentration of water molecules than in the dry state, but generally a lower concentration than in the conditioned or saturated states. Depending on configuration of the conduit parameters of the conduit, ambient conditions (including temperature and relative humidity of ambient air), and the concentration of water molecules within the breathable material of the elongate tube, expansion of the elongate tube may or may not be constrained by the sheath. In at least some examples, however, the conduit may be designed to have a spacing between the elongate tube and the sheath in the equilibrated state to allow for at least some expansion of the elongate tube before it is constrained by the sheath.

“Immersion testing” refers to a test for determining water absorption based on the ISO 62:2008(E) standard (© ISO, 2008), which is incorporated herein by reference in its entirety. First, at least three tubular test specimens are cut to a length of 25±1 mm from the elongate tube. The cuts should be made perpendicular to the longitudinal direction of the elongate tube. The cut edges should be smooth and free from cracks. The specimens should include only the active plastic(s) that is responsible for the water absorption properties. Heater wires, sleeves, and any mechanical support material should be removed non-destructively, if possible. Second, the specimens are dried. The specimens may be dried in a convection oven or vacuum oven maintained at 50±2° C. at for at least 24 hours, or in an industrial dryer at a temperature of 60° C., dew point of −40° C., air flow of 14 cubic meters per hour (m³/h) and drying time of 600 minutes (m). The specimens should be weighed regularly to the nearest 1 mg and returned to the oven/dryer until their mass is constant to within ±1 mg. Third, the specimens are allowed to cool to room temperature in a desiccator. Fourth, the specimens are weighed (m₁) and dimensions measured. Fifth, the specimens are immersed in distilled water for a period of 24 hours. There should be at least 8 ml of distilled water per square centimeter of the total surface are of the specimens, and no less than 300 ml per specimen. If necessary, the specimens may be placed in a stainless-steel wire basket connected to an anchor-weight by a stainless-steel wire. Sixth, the specimens are taken from the water and, using a lint free wipe, surface water is removed. Seventh, the specimens are weighed to the nearest 1 mg within 1 min of removing them from the water. Eighth, steps six (immersion) and seven (weighing) are repeated until the mass of the specimens is constant to within ±1 mg (m₂). Ninth, if the specimens are known or suspected to contain an appreciable amount of water-soluble ingredients, the second step (drying) is repeated and the samples weighed to correct for water-soluble matter lost during the immersion testing. If the reconditioned mass is less than the conditioned mass, the difference represents the water-soluble matter lost during the immersion testing. The water absorption of each specimen is expressed as the percentage change in mass c relative the initial mass, according to the to equation

$$c = \frac{{m}_{2} - {m}_{1}}{{m}_{1}} \times 100 \%$$

or, for a specimen containing water-soluble matter,

$$c = \frac{{m}_{2} - {m}_{3}}{{m}_{1}} \times 100 \% .$$

The result is expressed as the arithmetic mean of the three (or more) values obtained at the same exposure duration. References to immersion testing in this specification refers to testing of sample specimens of the breathable material or elongate tube alone, in isolation from the connectors and the sheath. That is, the connectors and the sheath are omitted or removed so that expansion is not constrained by the sheath.

“ISO” refers to the International Organization for Standardization, and more specifically to the international standards defined by the Organization. Those standards are subject to copyright and are available for purchase directly from the International Organization for Standardization at http://www.ISO.org.

“Leak,” “leakage” and “leakage testing” refer to Section 5.4 and the method set out in Annex E of the ISO 5367:2014 standard (@ ISO 2014), which is hereby incorporated by reference in its entirety. This standard defines limits for a complete breathing set or a conduit supplied ready for use with a ventilator breathing system (VBS) or anesthetic breathing system of 70 ml/min for an adult patient (intended delivered volume ≥300 ml), 40 ml/min for a pediatric patient (50<300 ml) or 30 ml/min for a neonatal patient (≤50 ml) at a pressure of 60±3 cmH₂O. For a single breathing tube not intended for use with a VBS or an anesthetic breathing system, the leakage limit is 25 ml/min at 60±3 cmH₂O. Briefly, leakage is tested according to the standard by first conditioning the conduit at a temperature of 23±3° C. for at least an hour. Second, one end of the conduit is closed off. Third, an internal gas pressure of 60±3 cmH₂O is applied and maintained. Fourth, the flow of air required to maintain that pressure is recorded. It will be appreciated that the conditioning defined by the standard may not reflect in use conditions and the standard was not drafted with breathable materials in mind. To better reflect in use conditions, leakage of a conduit formed at least in part from a breathable material may be further tested by additionally, or alternatively, conditioning the conduit to the simulated conditioned state as described above.

“Prolonged use,” “prolonged period of use,” “prolonged period” and the like refers to use of the conduit in a respiratory assistance system (or surgical insufflation system) conveying heated and humidified respiratory gases (or insufflation gas) for a continuous period of at least 24 hours. A prolonged period of use may be simulated by the simulated conditioned state described above.

“Resistance to flow” and “resistance to flow testing” refer to Section 5.5 and the method set out in the ISO 5367:2014 standard (@ ISO 2014), the entire content of which is hereby incorporated by reference in its entirety, at Annex F. This standard defines flow resistance limits for a conduit supplied ready to use of 0.06 cmH₂O/l/min at a flow of 30 l/min for an adult patient (intended delivered volume ≥300 ml), 0.12 cmH₂O/l/min at a flow of 15 l/min for a pediatric patient (50<300 ml) and 0.74 cmH₂O/l/min at a flow of 2.5 l/min. Briefly, resistance to flow is tested by first conditioning the conduit at a temperature of 23±3° C. for at least an hour. Second, the flow rate of a flow-controlling device is adjusted and maintained for 30 s and the pressure recorded. Third, the conduit is fitted over the outlet of a buffer reservoir and the free end of the conduit is secured so that the conduit is held straight. Fourth, the air flow is again adjusted and maintained for 30 s and the pressure recorded. Fifth, the increase in pressure due to the conduit is calculated from the difference of the recorded pressures. An increase in flow resistance with bending is tested by first conditioning the conduit at a temperature of 42±3° C. and relative humidity of at least 80% for at least one hour. Second, the conduit is suspended over a cylinder of 25 mm diameter and a tensile forces applied to maintain contact over half of the circumference of the cylinder. Third, the air flow is applied and the pressure recorded after five minutes. Fourth, the increase in pressure due to the conduit is calculated from the difference in pressure for the bent and straight conduits. It will be appreciated that the conditioning defined by the standard does may not reflect in use conditions and the standard was not drafted with breathable materials in mind. To better reflect in use conditions, resistance to flow of a conduit formed at least in part from a breathable material may be further tested by additionally, or alternatively, conditioning the conduit to the simulated conditioned state as described above.

“Saturated” refers to a state in which an elongate tube, or a sample thereof, has been subjected to immersion testing (i.e., submerged in liquid water) for a period of time until the permeable material absorbs no, or negligible, further water molecules. That is, until the combined mass of the permeable material and absorbed water molecules is at or near a maximum. It is an “artificial” state in that an elongate tube will not generally enter this state during normal use (i.e., in use in an assisted breathing system). The tube wall of an elongate tube in the saturated state generally contains a higher concentration of water molecules than in any of the dry, equilibrated and conditioned states.

### LISTING OF DRAWING ELEMENTS

100 respiratory assistance system
102 gases source
104 humidifier supply conduit
106 humidifier
108 inspiratory conduit
110 Y-piece
112 patient interface
114 expiratory conduit
116 pressure generator
118 gas inlet
120 gases source controller
122 user interface
124 gases source outlet
126 humidification chamber
128 chamber heater
130 humidifier controller
132 user interface
134 heater wire
136 sensor probe
138 sensor leads
140 gases return inlet
142 breathing circuit
144 inspiratory branch
146 expiratory branch
200 respiratory assistance system
202 heater wire
204 sensor
206 electropneumatic connector
208 supplementary gases inlet
302 heater base
304 cartridge
306 touchscreen display
308 switch
310 indicator light
312 release button
400 respiratory assistance system
402 pressure regulator
404 liquid
406 inlet probe
408 pressure relief valve
500 respiratory assistance system
502 blower
504 respiratory therapy device
506 housing
600 surgical insufflation system
602 insufflator supply conduit
604 insufflator
606 delivery conduit
608 surgical cannula
610 smoke evacuation system
612 wall source
614 compressed gas cylinder
616 discharge conduit
618 discharge filter
620 vacuum source
622 further discharge conduit
624 insufflation circuit
626 scope
628 laparoscopic monitor
700 conduit
702 elongate tube
704 sheath
706 connector
802 bore
902 longitudinal direction
904 clockwise braiding element
906 counter-clockwise braiding element
908 openings
1002 cuff portion
1004 first connector component
1006 second connector component
1008 chamfered edge
1010 flange
1012 annular channel
1014 aperture
1016 corrugated portion
1102 tapered conical end
1202 lumen
1502 spacing
1504 radial direction
1602 peak
1604 trough
1702 line
1704 melting point
1802 filament
2102 central portion
2104 end portion
2106 tapered portion
2108 unsheathed conduit
2202 corrugation
2204 side wall
2206 force vector
2402 depth
2602 interior narrowed waist region
2604 exterior narrowed waist regions
2700 respiratory assistance system
2702 filter
2704 inlet region
2706 outlet region
2708 intermediate region
2710 low point
2800 method of forming a conduit
2802 extrusion step
2804 corrugation step
2806 tube cutting step
2808 threading step
2810 connector insertion step
2812 clamping step
2814 connector overmolding step
2816 sheath contraction step
2818 sheath cutting step
2820 connector molding step

What is claimed is:

1. A medical gases conduit for use in a medical gases system, the medical gases conduit comprising:
an elongate tube defining a lumen for passage of a flow of medical gases, at least a portion of the elongate tube comprising a breathable material which, in use, is configured to expand due to absorption of water molecules; and
a sheath provided about at least a portion of the elongate tube, wherein the sheath is configured to constrain, in use, the expansion of at least a portion of the elongate tube in at least one of a radial direction and a longitudinal direction due to the absorption of the water molecules by the breathable material.

2. A medical gases conduit for use in a medical gases system, the medical gases conduit comprising:
an elongate tube defining a lumen for passage of a flow of medical gases, at least a portion of the elongate tube being corrugated and comprising:
a breathable material which, in use, is configured to expand due to absorption of water molecules, and
a plurality of corrugations; and
a sheath provided about at least a portion of the elongate tube and configured to, in use, constrain expansion of the elongate tube in at least a longitudinal direction of the elongate tube;
wherein the elongate tube is configured so that a pitch of the plurality of corrugations varies in use, in dependence upon the absorption of water molecules by the breathable material and constraint of the elongate tube by the sheath.

3. A medical gases conduit for use in a medical gases system, the medical gases conduit comprising:
an elongate tube defining a lumen for passage of a flow of medical gases, at least a portion of the elongate tube comprising a breathable material which, in use, is configured to expand due to absorption of water molecules; and
a sheath provided about at least a portion of the elongate tube, each end of the sheath secured to a corresponding end of the elongate tube, wherein the sheath is configured so that a length and a diameter of the sheath vary in inverse relation to each other as the breathable material expands due to the absorption of the water molecules, in use.

4. A medical gases conduit for use in a medical gases system, the medical gases conduit comprising:
an elongate tube defining a lumen for passage of a flow of medical gases;
a sheath provided about at least a portion of the elongate tube; and
a pair of connectors for pneumatically coupling the elongate tube to other components of the respiratory system, each of the pair of connectors securing a respective end of the sheath relative to a corresponding end of the elongate tube, and at least a first connector of the pair of connectors comprising a plurality of apertures through which one or more of the elongate tube and the sheath are exposed.

5. A medical gases conduit for use in a medical gases system, the medical gases conduit comprising:
an elongate tube defining a lumen for passage of a flow of medical gases, and
a sheath provided about at least a portion of the elongate tube,
wherein the sheath is configured to be at least partially spaced from a maximum outer diameter of the elongate tube when the medical gases conduit is in an equilibrated state.

6. A medical gases conduit for use in a medical gases system, the medical gases conduit comprising:
an elongate tube defining a lumen for passage of a flow of medical gases, at least a portion of the elongate tube comprising a breathable material which, in use, is configured to expand upon absorption of water molecules; and
a sheath provided about at least a portion of the elongate tube and configured to selectively constrain expansion of the elongate tube due to the absorption of the water molecules, in use;
wherein the elongate tube and the sheath are configured so that, in at least some conditions, a compliance of the medical gases conduit is dependent on a physical interaction between the elongate tube and the sheath.

7. A medical gases conduit for use in a medical gases system, the medical gases conduit comprising:
an elongate tube defining a lumen for passage of a flow of medical gases, at least a portion of the elongate tube comprising a breathable material which, in use, is configured to expand upon absorption of water molecules; and
a sheath provided loosely about at least a portion of the elongate tube;
wherein the elongate tube is configured to exert a hoop stress upon the sheath in at least some conditions, in use.

8. A medical gases conduit for use in a medical gases system, the medical gases conduit comprising:
an elongate tube defining a lumen for passage of a flow of medical gases; and
a sheath provided about at least a portion of the elongate tube, the sheath comprising a braided tubular mesh, the braided tubular mesh comprising a biaxial braid of a plurality of braiding elements, the plurality of braiding elements being spaced about a circumference of the sheath and extending helically about the elongate tube, and the plurality of braiding elements each comprising a pair of adjacent filaments.

9. A medical gases conduit for use in a medical gases system, the medical gases conduit comprising an elongate tube defining a lumen for passage of a flow of medical gases, at least a portion of the elongate tube comprising a breathable material, at least a portion of the elongate tube is configured to absorb between about 100% and 250%, preferably between about 100% and 180%, more preferably between about 110% and 150%, yet more preferably between about 120% and 140% or between about 130% and 145%, and yet more preferably between about 135% and 145%, e.g., about 138% or 139%, of its own dry mass in water molecules in immersion testing.

10. A medical gases conduit for use in a medical gases system, the medical gases conduit comprising an elongate tube defining a lumen for passage of a flow of medical gases, at least a portion of the elongate tube comprising a breathable material, at least a portion of the elongate tube is configured to absorb between about 40% and 80%, preferably between about 40% and 60%, more preferably between about 45% and 55%, and yet more preferably between about 48% and 51%, e.g., about 49%, of its own dry mass in water molecules in immersion testing, and the medical gases conduit does not comprise a heater wire.

11. A medical gases conduit for use in a medical gases system, the medical gases conduit comprising:
an extruded elongate tube defining a lumen for passage of a flow of medical gases, at least a portion of the elongate tube comprising a breathable material; and
a pair of connectors at respective ends of the elongate tube for pneumatically coupling the medical gases conduit with other components of the medical gases system, at least one of the pair of connectors comprising:
a first connector component inserted at least partially within the lumen of the elongate tube, and
a second connector component overmolded to the first connector component and the elongate tube.

12. A medical gases conduit for use in a medical gases system, the medical gases conduit comprising an elongate tube defining a lumen for passage of a flow of medical gases, at least a portion of the elongate tube comprising a breathable material, the breathable material in differential scanning calorimetry (DSC) testing comprising two melting points each at temperatures above about 197° C., preferably above about 202° C., more preferably above about 204° C., yet more preferably above about 205° C., and most preferably above about 206° C., e.g., at or above a temperature of about 207° C., wherein the medical gases conduit does not comprise a heater wire.

13. A medical gases conduit for use in a medical gases system, the medical gases conduit comprising an elongate tube defining a lumen for passage of a flow of medical gases, at least a portion of the elongate tube comprising a breathable material, the breathable material in differential scanning calorimetry (DSC) testing comprising three melting points, wherein the medical gases conduit does not comprise a heater wire.

14. A medical gases conduit for use in a medical gases system, the medical gases conduit comprising an elongate tube defining a lumen for passage of a flow of medical gases, at least a portion of the elongate tube comprising a breathable material, the breathable material in differential scanning calorimetry (DSC) testing comprising a lowest melting point at a temperature greater than about 37° C., preferably greater than about 42° C., more preferably greater than about 44° C., yet more preferably greater than about 45° C., and most preferably greater than about 46° C., e.g., at a temperature of about 47° C.

15. A medical gases conduit for use in a medical gases system, the medical gases conduit comprising an elongate tube defining a lumen for passage of a flow of medical gases, at least a portion of the elongate tube comprising a breathable material which, in use, is configured to expand due to absorption of water molecules, wherein the medical gases conduit is configured so that the elongate tube, in at least one conditioned state in use, has localized expansion in one or more of:
an inlet region, the inlet region comprising up to about 50%, preferably up to about 33%, more preferably up to about 25%, yet more preferably up to about 20%, and most preferably up to about 10% of a length of the elongate tube nearest a patient interface of the medical gases system;
an outlet region, the outlet region comprising up to about 50%, preferably up to about 33%, more preferably up to about 25%, yet more preferably up to about 20%, and most preferably up to about 10% of the length of the elongate tube nearest a gases return inlet of a gases source of the medical gases system; and
an intermediate region, the intermediate region comprising up to about 50%, preferably up to about 33%, more preferably up to about 25%, yet more preferably up to about 20%, and most preferably up to about 10% of the length of the elongate tube intermediate the inlet region and the outlet region.

16. The medical gases conduit of any one of claims 9 to 15, comprising a sheath provided about at least a portion of the elongate tube.

17. The medical gases conduit of any one of claim 4-5 or 8, the elongate tube comprising a breathable material.

18. The medical gases conduit of any one of claims 1 to 17, the medical gases conduit comprising an expiratory conduit, the medical gases system comprising a respiratory assistance system, and the flow of medical gases comprising a flow of respiratory gases.

19. The medical gases conduit of any one of claims 1 to 17, the medical gases conduit comprising an inspiratory conduit, the medical gases system comprising a respiratory assistance system, and the flow of medical gases comprising a flow of respiratory gases.

20. The medical gases conduit of any one of claims 1 to 17, the medical gases conduit comprising a discharge conduit, the medical gases system comprising a surgical insufflation system, and the flow of medical gases comprising a flow of insufflation gas.

21. The medical gases conduit of any one of claims 1 to 20, configured so that a diameter and a length of at least one, and preferably both, of the elongate tube and the sheath may vary temporally, in use.

22. The medical gases conduit of any one of claims 1 to 21, wherein a length and a diameter of the sheath are configured to vary in inverse relation as the elongate tube expands due to absorption of water molecules, in use.

23. The medical gases conduit of any one of claims 1 to 22, wherein a length vs diameter gradient of the sheath is between about −100 and −20, preferably between about −50 and −20, more preferably between about −40 and −35, and is most preferably about −37.

24. The medical gases conduit of any one of claims 1 to 22, wherein a length vs diameter gradient of the sheath is between about −75 and −25, preferably between about −60 and −40, more preferably between about −55 and −45, and is most preferably about −52.

25. The medical gases conduit of any one of claims 1 to 24, wherein a length vs diameter gradient of the sheath is substantially linear between sheath diameters of about 24 mm and 32 mm.

26. The medical gases conduit of any one of any one of claims 1 to 25, wherein the sheath, in isolation, is configured to conform to rods with outside diameters ranging between about 23 mm and 43 mm.

27. The medical gases conduit of any one of claims 1 to 26, the sheath comprising a plurality of openings, preferably quadrilateral openings, which are configured to undergo an affine transformation as the elongate tube expands due to the absorption of the water molecules by the breathable material, in use.

28. The medical gases conduit of any one of claims 1 to 27, wherein the sheath is configured to be at least partially spaced from the elongate tube when the medical gases conduit is in at least one, and preferably both, of a dry state and an equilibrated state.

29. The medical gases conduit of any one of claims 1 to 28, wherein the elongate tube and the sheath are configured so that, in at least one cross-section through the medical gases conduit, the sheath is spaced outwardly from at least part of the circumference of the elongate tube in any one or more of a dry state, an equilibrated state, and at least one conditioned state, in use.

30. The medical gases conduit of any one of claims 1 to 29, wherein the elongate tube and the sheath are configured so that the elongate tube exerts a hoop stress upon the sheath in at least one conditioned state, in use.

31. The medical gases conduit of any one of claims 1 to 30, wherein the elongate tube and the sheath are configured so that, in at least one cross-section through the medical gases conduit, the sheath contacts an entire circumference of the elongate tube in at least one conditioned state, in use.

32. The medical gases conduit of any one of claims 1 to 31, wherein the breathable material is unfoamed.

33. The medical gases conduit of any one of claims 1 to 31, wherein the breathable material is foamed.

34. The medical gases conduit of any one of claims 1 to 33, the breathable material comprising a block copolymer, preferably a block copolymer comprising one or more of:
hard segments of polybutylene terephthalate; and
soft segments of an ether type macro glycol.

35. The medical gases conduit of claim 34, the block copolymer comprising at least about 90%, preferably at least about 92%, more preferably at least about 95%, yet more preferably at least about 97%, e.g., about 97% or about 98.5% of the breathable material by one or more of mass, weight and volume.

36. The medical gases conduit of any one of claims 1 to 35, the breathable material comprising one or more additives, e.g., one or more of:
a foaming agent,
a colorant,
an ultraviolet (UV) stabilizer,
a UV absorber, and
a processing aid.

37. The medical gases conduit of claim 36, the one or more additives comprising up to about 10%, preferably up to about 8%, more preferably up to about 5%, and most preferably up to about 3%, e.g., about 3% or about 2% of the breathable material, by one or more of mass, weight and volume.

38. The medical gases conduit of any one of claims 1 to 37, wherein at least a portion of the elongate tube is configured to absorb more than about 33%, more preferably between about 33% and 200%, yet more preferably between about 100% and 160%, yet more preferably between about 120% and 140%, and most preferably between about 130% and 135%, e.g., about 133%, of its own dry mass in water molecules, in immersion testing.

39. The medical gases conduit of any one of claims 1 to 38, wherein at least a portion of the elongate tube is configured to absorb between about 45% and 250%, preferably between about 65% and 200%, more preferably between about 75% and 175%, yet more preferably between about 100% and 160%, yet more preferably between about 110% and 150%, yet more preferably between about 120% and 140%, yet more preferably between about 130% and 140%, and most preferably between about 133% and 139%, e.g., about 139%, of its own dry mass in water molecules, in immersion testing.

40. The medical gases conduit of any one of claims 1 to 39 wherein, in immersion testing in isolation from the sheath, at least a portion of the elongate tube is configured to expand by between about 20% and 70%, preferably between about 25% and 50%, and more preferably between about 30% and 50%, e.g., by about 32%, in one or more, and preferably each, of the radial direction, the longitudinal direction, and a wall thickness in immersion testing.

41. The medical gases conduit of any one of claims 1 to 40 wherein, in immersion testing in isolation from the sheath, at least a portion of the elongate tube is configured to expand by one or more, and preferably all, of:
between about 37% and 47%, preferably about 42%, in the radial direction;
between about 32% and 42%, preferably about 37%, in the longitudinal direction; and/or
between about 29% and 39%, preferably about 34%, in the wall thickness.

42. The medical gases conduit of any one of claims 1 to 41 comprising a pair of connectors at opposing ends of the medical gases conduit, the pair of connectors configured for pneumatically coupling the elongate tube with other components of the medical gases system, at least one of the pair of connectors comprising a first connector component and a second connector component, wherein corresponding ends of the elongate tube and the sheath are secured between the first connector component and the second connector component.

43. The medical gases conduit of claim 42, the first connector component extending into the lumen of the elongate tube.

44. The medical gases conduit of claim 42 or 43 wherein the second connector component is overmolded to one or more, and preferably all, of the first connector component, the elongate tube and the sheath.

45. The medical gases conduit of any one of claims 42 to 44, the second connector component comprising a plurality of apertures, preferably between two and six apertures, e.g., four apertures, through which the elongate tube and the sheath are exposed.

46. The medical gases conduit of any one of claims 42 to 45, wherein:
an end of the elongate tube is disposed substantially concentrically about a portion of the first connector component;
an end of the sheath is disposed substantially concentrically about the end of the elongate tube; and
the second connector component is disposed substantially concentrically about the end of the sheath.

47. The medical gases conduit of any one of claims 42 to 46, wherein at least one of the first connector component and the second connector component is formed at least in part from one or more of polycarbonate, polypropylene and polyethylene.

48. The medical gases conduit of any one of claims 42 to 47, wherein the at least one of the pair of connectors is configured to prevent or inhibit expansion of the end of the elongate tube secured between the first connector component and the second connector component.

49. The medical gases conduit of any one of claims 42 to 48, wherein the elongate tube and the sheath are not secured to each other intermediate the pair of connectors.

50. The medical gases conduit of any one of claims 42 to 49, wherein the conduit is configured so that the elongate tube tapers towards at least one, and preferably both, of the pair of connectors in at least one conditioned state, in use.

51. The medical gases conduit of any one of claims 42 to 50, wherein the conduit is configured so that the sheath tapers towards at least one, and preferably both, of the pair of connectors in at least one of, and preferably both, an equilibrated state and a conditioned state.

52. The medical gases conduit of any one of claims 1 to 51 wherein the medical gases conduit is configured so that, in use:
upon initially absorbing water molecules, the elongate tube is configured to expand relatively unconstrained by the sheath, and
upon further absorption of water molecules, the elongate tube is configured to expand relatively constrained by the sheath.

53. The medical gases conduit of claim 52, wherein the elongate tube is configured to expand relatively constrained by the sheath after prolonged use.

54. The medical gases conduit of any one of claims 1 to 53, wherein the sheath is configured to:
not constrain expansion of the elongate tube when the elongate tube is in an equilibrated state, in use; and
constrain expansion of the elongate tube when the elongate tube is in at least one conditioned state, in use.

55. The medical gases conduit of any one of claims 1 to 54 wherein the medical gases conduit is configured so that, in use:
upon initially absorbing water molecules, the elongate tube is configured to expand in the radial direction relatively unconstrained by the sheath, and
upon further absorption of water molecules, the elongate tube is configured to further expand in the radial direction relatively constrained by the sheath.

56. The medical gases conduit of any one of claims 1 to 55, wherein the sheath is configured to:
not constrain expansion of the elongate tube in the radial direction when the elongate tube is in an equilibrated state; and
constrain expansion of the elongate tube in the radial direction when the elongate tube is in at least one conditioned state, in use.

57. The medical gases conduit of any one of claims 1 to 56 wherein the medical gases conduit is configured so that, in use:
upon initially absorbing water molecules, the elongate tube is configured to expand in the longitudinal direction relatively unconstrained by the sheath; and
upon further absorption of water molecules, the elongate tube is configured to expand in the longitudinal direction relatively constrained by the sheath.

58. The medical gases conduit of claim 57, wherein the medical gases conduit is configured so that, upon yet further absorption of water molecules in use, the elongate tube is configured to expand in the radial direction and the sheath is configured to cause the medical gases conduit to at least partially contract in the longitudinal direction.

59. The medical gases conduit of any one of claims 1 to 58, wherein the sheath is configured to not constrain expansion of the elongate tube in the longitudinal direction in an equilibrated state, and to constrain expansion of the elongate tube in the longitudinal direction in at least one conditioned state.

60. The medical gases conduit of any one of claims 1 to 59 wherein the medical gases conduit is configured so that, in use:
upon initially absorbing water molecules, the elongate tube is configured to freely expand in the radial direction and the longitudinal direction, and
upon further absorption of water molecules, the elongate tube is configured to engage the sheath and further expansion is constrained in at least one of the radial direction and the longitudinal direction.

61. The medical gases conduit of any one of claims 1 to 60, wherein the sheath is configured to allow the elongate tube to expand freely in the radial direction and the longitudinal direction in an equilibrated state, and configured to constrain expansion of the elongate tube in at least one of the radial direction and the longitudinal direction in at least one conditioned state.

62. The medical gases conduit of any one of claims 1 to 61, wherein the medical gases conduit is configured so that, in use:
upon initially absorbing water molecules, the elongate tube is configured to freely expand in a radial direction to engage an inner surface of the sheath, and
upon further absorption of water molecules, the elongate tube is configured to further expand in the radial direction, causing the sheath to expand in the radial direction and contract in the longitudinal direction.

63. The medical gases conduit of any one of claims 1 to 62, wherein the elongate tube and the sheath are configured so that expansion of the elongate tube in the radial direction causes the elongate tube to engage the sheath, and the sheath to expand in the radial direction and contract in the longitudinal direction.

64. The medical gases conduit of any one of claims 1 to 63, wherein the elongate tube is configured to have a first length prior to use, a second length upon initially absorbing water molecules, and a third length upon further absorption of water molecules, wherein the second length is longer than the first length and the third length.

65. The medical gases conduit of any one of claims 1 to 64, wherein the elongate tube is configured to have a first length in an equilibrated state, a second length in at least one conditioned state in which radial expansion of the elongate tube is not constrained by the sheath, and a third length in at least one other conditioned state in which radial expansion of the elongate tube is constrained by the sheath, wherein the second length is longer than the first length and the third length.

66. The medical gases conduit of claim 64 or 65, wherein the third length is within about 90% to 110% of the first length, and preferably about equal to the first length.

67. The medical gases conduit of any one of claims 1 to 66, the elongate tube comprising a plurality of corrugations.

68. The medical gases conduit of claim 67, wherein the medical gases conduit is configured so that a profile of each of the plurality of corrugations changes as the elongate tube expands due to absorption of water molecules, in use.

69. The medical gases conduit of claim 67 or 68, wherein the plurality of corrugations are each defined in part by a pair of side walls, and the elongate tube and the sheath are configured so that the side walls reorient, preferably towards the radial direction, as the elongate tube absorbs water molecules, in use.

70. The medical gases conduit of claim 69, wherein the side walls are configured to reorient beyond a radial direction as the elongate tube continues to absorb water molecules.

71. The medical gases conduit of any one of claims 67 to 70, wherein the medical gases conduit is configured so that a pitch of the plurality of corrugations is substantially uniform along a length of the elongate tube when the elongate tube is in at least one of a dry state and an equilibrated state, and the pitch of the plurality of corrugations varies along the length of the elongate tube when the elongate tube is in at least one conditioned state.

72. The medical gases conduit of any one of claims 1 to 71, the elongate tube comprising a cuff portion at an end of the elongate tube, wherein the cuff portion is uncorrugated.

73. The medical gases conduit of any one of claims 1 to 72, the sheath comprising a tubular mesh, preferably a braided tubular mesh.

74. The medical gases conduit of any one of claims 1 to 73, the sheath comprising a biaxial braid of a plurality of braiding elements each extending helically about the elongate tube.

75. The medical gases conduit of claim 74, the plurality of braiding elements each comprising two or three filaments.

76. The medical gases conduit of claim 74 or 75, the sheath comprising at least one of:
between about 75 and 125 braiding elements, preferably between about 90 and 100 braiding elements, e.g., about 96 braiding elements; and
between about 150 and 250 filaments, and preferably between about 180 and 200 filaments, e.g., about 192 filaments.

77. The medical gases conduit of any one of claims 1 to 76, wherein the elongate tube is configured to have:
a substantially uniform diameter in at least one of a dry state and an equilibrated state; and
a substantially non-uniform diameter in at least one conditioned state, in use.

78. The medical gases conduit of any one of claims 1 to 77, wherein the medical gases conduit is configured so that the elongate tube, in at least one conditioned state in use, has localized expansion in one or more regions of elevated relative humidity or volume of condensate within the lumen.

79. The medical gases conduit of any one of claims 1 to 78, wherein the medical gases conduit is an expiratory conduit configured so that the elongate tube, in a conditioned state in use, has localized expansion in one or more of:
an inlet region, the inlet region comprising up to about 50%, preferably up to about 33%, more preferably up to about 25%, yet more preferably up to about 20%, and most preferably up to about 10% of a length of the elongate tube nearest a patient interface of the medical gases system;
an outlet region, the outlet region comprising up to about 50%, preferably up to about 33%, more preferably up to about 25%, yet more preferably up to about 20%, and most preferably up to about 10% of the length of the elongate tube nearest a gases return inlet of a gases source of the medical gases system; and
an intermediate region, the intermediate region comprising up to about 50%, preferably up to about 33%, more preferably up to about 25%, yet more preferably up to about 20%, and most preferably up to about 10% of the length of the elongate tube intermediate the inlet region and the outlet region.

80. The medical gases conduit of any one of claims 1 to 79, the breathable material in differential scanning calorimetry (DSC) testing comprising a lowest melting point at a temperature greater than about 37° C., preferably greater than about 42° C., more preferably greater than about 44° C., more preferably greater than about 45° C., and most preferably greater than about 46° C., e.g., at a temperature of about 47° C.

81. The medical gases conduit of any one of claims 1 to 80, the breathable material in DSC testing comprising a lowest melting point at a temperature between about 37° C. and 100° C., preferably between about 40° C. and 60° C., and more preferably between about 40° C. and 50° C., e.g., at a temperature of about 47° C.

82. The medical gases conduit of any one of claims 1 to 81, the breathable material in differential scanning calorimetry (DSC) testing comprising a melting point at a temperature between about 42° C. and 52° C., preferably between about 44° C. and 50° C., more preferably between about 45° C. and 49° C., and yet more preferably between about 46° C. and 47° C., e.g., at a temperature of about 47° C.

83. The medical gases conduit of any one of claims 1 to 82, the breathable material in differential scanning calorimetry (DSC) testing comprising two melting points at temperatures above about 197° C., preferably above about 202° C., more preferably above about 204° C., yet more preferably above about 205° C., and most preferably above about 206° C., e.g., at or above a temperature of about 207° C.

84. The medical gases conduit of any one of claims 1 to 83, the breathable material in differential scanning calorimetry (DSC) testing comprising two melting points at temperatures between about 202° C. and 225° C., preferably between about 204° C. and 223° C., more preferably between about 205° C. and 222° C., and yet more preferably between about 206° C. and 221° C., e.g., at temperatures of about 207° C. and 220° C.

85. The medical gases conduit of any one of claims 1 to 84, the breathable material in differential scanning calorimetry (DSC) testing comprising two melting points with a temperature difference of less than about 23° C., preferably less than about 18° C., more preferably less than about 16° C., yet more preferably less than about 15° C., and most preferably less than about 14° C., e.g., with a temperature difference of about 13° C.

86. The medical gases conduit of any one of claims 1 to 85, the breathable material in differential scanning calorimetry (DSC) testing comprising two melting points with a temperature difference of between about 150° C. and 170° C., preferably between about 155° C. and 165° C., more preferably between about 158° C. and 162° C., and yet more preferably between about 159° C. and 161° C., e.g., a temperature difference of about 160° C.

87. The medical gases conduit of any one of claims 1 to 86, the breathable material in differential scanning calorimetry (DSC) testing comprising three melting points at temperatures between about 42° C. and 225° C., more preferably between about 44° C. and 223° C., yet more preferably between about 45° C. and 222° C., and most preferably between about 46° C. and 221° C.

88. The medical gases conduit of any one of claims 1 to 87, the breathable material in differential scanning calorimetry (DSC) testing comprising melting point(s) at temperature(s) of at least one, and preferably all three, of:
between about 42° C. and 52° C., preferably between about 44° C. and 50° C., more preferably between about 45° C. and 49° C., and yet more preferably between about 46° C. and 47° C., e.g., at a temperature of about 47° C.;
between about 202° C. and 212° C., preferably between about 204° C. and 210° C., more preferably between about 205° C. and 209° C., and yet more preferably between about 206° C. and 208° C., e.g., at a temperature of about 207° C.; and
between about 215° C. and 225° C., preferably between about 217° C. and 223° C., more preferably between about 218° C. and 222° C., and yet more preferably between about 219° C. and 221° C., e.g., at a temperature of about 220° C.

89. The medical gases conduit of any one of claims 1 to 88, wherein the medical gases conduit is configured to have a compliance of less than about 4 ml/cmH₂O, preferably less than about 2.5 ml/cmH₂O, and more preferably less than about 1.2 ml/cmH₂O in at least one conditioned state.

90. The medical gases conduit of any one of claims 1 to 89, wherein the elongate tube, in isolation from the sheath, is configured to have a compliance of more than about 4 ml/cmH₂O, preferably more than 5 ml/cmH₂O, in at least one conditioned state.

91. The medical gases conduit of any one of claims 1 to 90, wherein the medical gases conduit is configured to have a resistance to flow, in at least one conditioned state, of less than about:
0.06 cmH₂O/l/min at a flow of 30 l/min;
0.12 cmH₂O/l/min at a flow of 15 l/min; or
0.74 cmH₂O/l/min at a flow of 2.5 l/min.

92. The medical gases conduit of any one of claims 1 to 91, wherein the medical gases conduit is configured to have an increase in flow resistance with bending of less than 150% in at least one conditioned state.

93. The medical gases conduit of any one of claims 1 to 92, the medical gases conduit configured to comply with any one or more, and preferably all, of the length, leakage, resistance to flow and compliance requirements of the International Organization for Standardization (ISO)'s International Standard 5367:2014(E) standard in at least one conditioned state.

94. The medical gases conduit of any one of claims 1 to 93, wherein the medical gases conduit is configured to have a compliance of less than about 4 ml/cmH₂O, preferably less than about 2.5 ml/cmH₂O, and more preferably less than about 1.2 ml/cmH₂O after prolonged use.

95. The medical gases conduit of any one of claims 1 to 94, wherein the elongate tube, in isolation from the sheath, is configured to have a compliance of more than about 4 ml/cmH₂O, preferably more than 5 ml/cmH₂O after prolonged use.

96. The medical gases conduit of any one of claims 1 to 95, wherein the medical gases conduit is configured to have a resistance to flow, after prolonged use, of less than about:
0.06 cmH₂O/l/min at a flow of 30 l/min;
0.12 cmH₂O/l/min at a flow of 15 l/min; or
0.74 cmH₂O/l/min at a flow of 2.5 l/min.

97. The medical gases conduit of any one of claims 1 to 96, wherein the medical gases conduit is configured to have an increase in flow resistance with bending of less than 150% after prolong use.

98. The medical gases conduit of any one of claims 1 to 97, the medical gases conduit is configured to comply with any one or more, and preferably all, of the length, leakage, resistance to flow and compliance requirements of the International Organization for Standardization (ISO)'s International Standard 5367:2014(E) after prolonged use.

99. The medical gases conduit of any one of claims 1 to 98, wherein the elongate tube and the sheath are flexible to reduce tube drag forces one or more of a Y-piece and a patient interface of the medical gases system, in use.

100. The medical gases conduit of any one of claims 1 to 99, wherein the elongate tube, in isolation from the sheath, is more flexible in a conditioned state than in an equilibrated state.

101. The medical gases conduit of any one of claims 1 to 100, the medical gases conduit comprising an expiratory conduit, the medical gases system comprising a respiratory assistance system, and the flow of medical gases comprising a flow of respiratory gases, wherein the expiratory conduit is configured to form upwards of about 80%, preferably upwards of about 90%, and more preferably upwards of about 95%, of an overall length of an expiratory branch of the respiratory assistance system, in use.

102. The medical gases conduit of any one of claims 1 to 101, wherein the medical gases conduit or the elongate tube, in an equilibrated state, has a length of:
between about 0.8 m and 1.2 m, e.g., about 1.0 m;
between about 1.0 m and 2.5 m, and preferably:
between about 1.1 m and 1.4 m, and more preferably between about 1.2 m and 1.3 m, e.g., about 1.25 m;
between about 1.4 m and 1.6 m, e.g., about 1.5 m;
between about 1.5 m and 1.7 m, e.g., about 1.6 m; or
between about 1.5 m and 1.8 m, e.g., about 1.6 m or about 1.8 m; or
between about 2.2 m and 2.6 m, more preferably between about 2.3 m and 2.5 m, e.g., about 2.4 m.

103. The medical gases conduit of any one of claims 1 to 102, the medical gases conduit measuring between about 1.1 m and 1.8 m, preferably between about 1.2 m and 1.8 m, more preferably between about 1.2 m and 1.3 m or between about 1.4 m and 1.6 m or between about 1.5 m and 1.7 m, e.g., about 1.2 m or 1.5 m or 1.6 m, in length, in an equilibrated state.

104. The medical gases conduit of any one of claims 1 to 103, wherein a wall thickness of the elongate tube, in a dry state, is between about 0.5 mm and 0.9 mm, preferably between about 0.6 mm and 0.8 mm, more preferably between about 0.65 mm and 0.75 mm, yet more preferably between about 0.68 mm and 0.72 mm, and most preferably between about 0.69 mm and 0.71 mm, e.g., about 0.70 mm.

105. The medical gases conduit of any one of claims 1 to 104, wherein a wall thickness of the elongate tube, in a saturated state in isolation from the sheath, is between about 0.7 mm and 1.1 mm, preferably between about 0.8 mm and 1.0 mm, more preferably between about 0.85 mm and 0.95 mm, yet more preferably between about 0.90 mm and 0.94 mm, and most preferably between about 0.91 mm and 0.93 mm, e.g., about 0.92 mm.

106. The medical gases conduit of any one of claims 1 to 105, wherein a maximum outer diameter of the elongate tube, in a dry state, is between about 20 mm and 26 mm, preferably between about 21 mm and 25 mm, and more preferably between about 22 mm and 24 mm, e.g., about 23 mm.

107. The medical gases conduit of any one of claims 1 to 106, wherein a maximum outer diameter of the elongate tube, in a saturated state in isolation from the sheath, is between about 25 mm and 35 mm, preferably between about 28 mm and 32 mm, and more preferably between about 29 mm and 31 mm, e.g., about 30 mm.

108. The medical gases conduit of any one of claims 1 to 107, wherein the elongate tube is extruded.

109. The medical gases conduit of any one of claims 1 to 108, wherein the elongate tube is corrugated.

110. The medical gases conduit of any one of claims 1 to 109, wherein the elongate tube is corrugated and the sheath is configured to be at least partially spaced from peaks of corrugations of the elongate tube when the medical gases conduit is in an equilibrated state.

111. The medical gases conduit of any one of claims 1 to 110, wherein the medical gases conduit does not comprise a reinforcing spine or rod within the lumen.

112. The medical gases conduit of any one of claims 1 to 111, wherein the breathable material is configured to at least partially dry the flow of medical gases as they pass along a length of the lumen, in use.

113. The medical gases conduit of any one of claims 1 to 112, wherein the at least one conditioned state comprises a simulated conditioned state.

114. The medical gases conduit of any one of claims 1 to 113, the medical gases system comprising a Y-piece and a gases source, the medical gases conduit configured to be coupled directly to each of:
an outlet of the Y-piece; and
a gases return inlet of the gases source, or a filter coupled directly to the gases return inlet.

115. The medical gases conduit of any one of claims 1 to 114, wherein the medical gases conduit does not comprise a water trap or provision for a water trap.

116. The medical gases conduit of any one of claims 1 to 115, configured for use as or in an expiratory branch of the medical gases system, wherein the expiratory branch does not comprise a water trap.

117. The medical gases conduit of any one of claims 1 to 116, wherein the medical gases conduit does not comprise a heater wire.

118. A breathing circuit kit for use in a respiratory assistance system, the breathing circuit kit comprising:
an inspiratory conduit;
a Y-piece; and
an expiratory conduit, at least one of the inspiratory conduit and the expiratory conduit comprising the medical gases conduit according to any one of claims 1 to 117.

119. The breathing circuit kit of claim 118, comprising a humidification chamber.

120. The breathing circuit kit of claim 118 or 119, comprising any one or more of:
a humidifier supply conduit;
a patient interface;
a filter;
a pressure relief valve;
a pressure regulator; and
a catheter mount.

121. The breathing circuit kit of any one of claims 118 to 120, the breathing circuit kit not comprising at least one, and preferably both, of:
a heater wire associated with the medical gases conduit; and
a water trap associated with the medical gases conduit.

122. An insufflation circuit kit comprising:
a discharge conduit comprising the medical gases conduit of any one of claims 1 to 117; and
a discharge filter.

123. The insufflation circuit kit of claim 122, comprising any one or more of:
an insufflator supply conduit;
an adapter;
a humidifier supply conduit;
an insufflation gas filter;
a humidification chamber;
a funnel;
a delivery conduit;
a surgical cannula;
a diffuser; and
a further discharge conduit.

124. A respiratory assistance system for use in providing respiratory therapy to a patient, the respiratory assistance system comprising:
a gases source configured to supply a flow of respiratory gases;
a humidifier configured to heat and humidify the flow of respiratory gases;
a humidifier supply conduit configured convey the flow of respiratory gases from an outlet of the gases source to an inlet of the humidifier;
an inspiratory conduit configured to convey the flow of respiratory gases from an outlet of the humidifier to an inlet of a Y-piece or a patient interface for supply to the patient; and
an expiratory conduit configured to convey the flow of medical gases from an outlet of the Y-piece or the patient interface to a gases return inlet of the gases source or a filter at the gases return inlet;
at least one of the inspiratory conduit and the expiratory conduit comprising the medical gases conduit of any one of claims 1 to 117.

125. The respiratory assistance system of claim 124, the respiratory assistance system not comprising at least one, and preferably both, of:
a heater wire associated with the medical gases conduit; and
a water trap associated with the medical gases conduit.

126. A surgical insufflation system configured to supply an insufflation gas to a body cavity of a patient, the surgical insufflation system comprising:
an insufflator configured to supply a flow of insufflation gas;
a humidifier configured to heat and humidify the flow of insufflation gas;
a humidifier supply conduit configured to convey the flow of insufflation gas from an outlet of the insufflator to an inlet of the humidifier;
a delivery conduit configured to convey the flow of insufflation gas from an outlet of the humidifier to an inlet of a surgical cannula for supply to the body cavity of the patient; and
a discharge conduit configured to convey the flow of insufflation gas and surgical smoke away from the body cavity of the patient, the discharge conduit comprising the medical gases conduit of any one of claims 1 to 117.

127. A method of forming a conduit for use in conveying medical gases, the method comprising:
providing a sheath about an elongate tube, the elongate tube defining a lumen for passage of a flow of medical gases;
clamping a first end of the sheath to a first end of the elongate tube; and
overmolding a connector to the first end of the sheath and the first end of the elongate tube to secure the connector, the sheath and the elongate tube together.

128. The method of claim 127, the step of overmolding the connector comprising:
inserting a first connector component at least partially into the lumen of the elongate tube wall at the first end of the elongate tube; and
overmolding a second connector component about the first connector component, the first end of the sheath and the first end of the elongate tube.

129. The method of claim 128, comprising a step of injection molding the first connector component prior to inserting the first connector component at least partially into the first end of the elongate tube.

130. The method of any one of claims 127 to 129, the step of clamping the first end of the sheath to the first end of the elongate tube comprising positioning the first end of the sheath and the first end of the elongate tube within a mold tool, the mold tool comprising a plurality of protrusions engaging and clamping the sheath to the elongate tube.

131. The method of claim 130, the plurality of protrusions forming a plurality of apertures in the connector upon overmolding the connector, wherein the sheath is exposed through the plurality of apertures.

132. The method of any one of claims 127 to 131, comprising steps of:
clamping a second end of the sheath to a second end of the elongate tube; and
overmolding a second connector to the second end of the sheath and the second end of the elongate tube to secure the second connector, the second end of the sheath and the second end of the elongate tube together.

133. The method of claim 132, comprising a step of contracting the sheath in a longitudinal direction so that the second end of the sheath overlies the second end of the elongate tube, prior to clamping the second end of the sheath to the second end of the elongate tube.

134. The method of any one of claims 127 to 133, comprising a step of extruding the elongate tube.

135. The method of claim 134, comprising a step of corrugating the elongate tube.

136. The method of claim 134 or 135, comprising a step of cutting the elongate tube from a tubular extrusion.

137. The method of any one of claims 127 to 136, comprising a step of cutting the sheath to a predetermined sheath length from a length of sheathing, wherein the predetermined sheath length is selected so that the sheath is at least partially spaced from the elongate tube in a radial direction when the sheath is provided about the elongate tube and the sheath is deformed to a length of the elongate tube.

138. The method of any one of claims 127 to 137, the sheath comprising a tubular mesh, preferably a braided tubular mesh.

139. The method of any one of claims 127 to 138, the sheath comprising at least one of:
between about 75 and 125 monofilament or multifilament braiding elements, preferably between about 90 and 100 braiding elements, e.g., about 96 braiding elements; and
between about 150 and 250 filaments, and preferably between about 180 and 200 filaments, e.g., about 192 filaments.

140. The method of any one of claims 127 to 138, the sheath comprising at least one of:
between about 40 and 60 monofilament or multifilament braiding elements, preferably between about 45 and 55 braiding elements, e.g., about 48 braiding elements; and
between about 120 and 180 filaments, and preferably between about 135 and 165 filaments, e.g., about 144 filaments.

141. The method of any one of claims 127 to 140, the elongate tube comprising a breathable material, preferably a block copolymer, and more preferably a block copolymer comprising:
hard segments of polybutylene terephthalate; and
soft segments of an ether type macro glycol.

142. The method of any one of claims 127 to 141, not comprising a step of providing at least one, and preferably both, of a heater wire and a water trap to the conduit.

143. A conduit formed by the method of any one of claims 127 to 142, wherein the elongate tube is configured to expand upon absorbing water molecules and the sheath is configured to selectively constrain expansion of the elongate tube, in use.
